(19) United States
(12) Patent Application Publication
(10) Pub. No.: US 2021/0276977 A1
(43) Pub. Date: Sep. 9, 2021

(54) BICYCLIC INHIBITORS OF HISTONE DEACETYLASE

(71) Applicant: Alkermes, Inc., Waltham, MA (US)

(72) Inventors: Nathan Oliver Fuller, Arlington, MA (US); John A. Lowe, III, Stonington, CT (US)

(21) Appl. No.: 17/260,192

(22) PCT Filed: Jul. 12, 2019
(86) PCT No.: PCT/US2019/041587

§ 371 Date: Jan. 13, 2021

Related U.S. Application Data
(60) Provisional application No. 62/697,497, filed on Jul. 13, 2018.

(51) Int. Cl. C07D 401/14; C07D 471/04; C07D 417/14; C07D 413/14

(52) U.S. Cl. CPC C07D 401/14; C07D 413/14; C07D 417/14; C07D 471/04

(57) ABSTRACT

Provided herein are compounds of Formula I and pharmaceutically acceptable salts and compositions thereof, which are useful for treating a variety of conditions associated with histone deacetylases (HDAC).

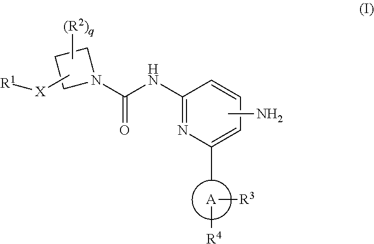

### RELATED APPLICATIONS

This application claims the benefit of priority to U.S. Provisional Application No. 62/697,497, filed Jul. 13, 2018, the entire contents of which are incorporated herein by reference.

### BACKGROUND

Inhibitors of histone deacetylases (HDAC) have been shown to modulate transcription and to induce cell growth arrest, differentiation and apoptosis. HD AC inhibitors also enhance the cytotoxic effects of therapeutic agents used in cancer treatment, including radiation and chemotherapeutic drugs. Marks, P., Rifkind, R. A., Richon, V. M., Breslow, R., Miller, T., Kelly, W. K. Histone deacetylases and cancer: causes and therapies. Nat Rev Cancer, 1, 194-202, (2001); and Marks, P. A., Richon, V. M., Miller, T., Kelly, W. K. Histone deacetylase inhibitors. Adv Cancer Res, 91, 137-168, (2004). Moreover, recent evidence indicates that transcriptional dysregulation may contribute to the molecular pathogenesis of certain neurodegenerative disorders, such as Huntington's disease, spinal muscular atrophy, amyotropic lateral sclerosis, and ischemia. Langley, B., Gensert, J. M., Beal, M. F., Ratan, R. R. Remodeling chromatin and stress resistance in the central nervous system: histone deacetylase inhibitors as novel and broadly effective neuroprotective agents. Curr Drug Targets CNS Neurol Disord, 4, 41-50, (2005). A recent review has summarized the evidence that aberrant histone acetyltransferase (HAT) and histone deacetylases (HDAC) activity may represent a common underlying mechanism contributing to neurodegeneration. Moreover, using a mouse model of depression, Nestler has recently highlighted the therapeutic potential of histone deacetylation inhibitors (HDAC5) in depression. Tsankova, N. M., Berton, O., Renthal, W., Kumar, A., Neve, R. L., Nestler, E. J. Sustained hippocampal chromatin regulation in a mouse model of depression and antidepressant action. Nat Neurosci, 9, 519-525, (2006).

There are 18 known human histone deacetylases, grouped into four classes based on the structure of their accessory domains. Class I includes HDAC1, HDAC2, HDAC3, and HD AC 8 and has homology to yeast RPD3. HDAC4, HDAC5, HDAC7, and HDAC9 belong to class IIa and have homology to yeast. HDAC6 and HD AC 10 contain two catalytic sites and are classified as class lib. Class III (the sirtuins) includes SIRT1, SIRT2, SIRT3, SIRT4, SIRT5, SIRT6, and SIRT7. HDAC11 is another recently identified member of the HD AC family and has conserved residues in its catalytic center that are shared by both class I and class II deacetylases and is sometimes placed in class IV.

In contrast, HDACs have been shown to be powerful negative regulators of long-term memory processes. Nonspecific HD AC inhibitors enhance synaptic plasticity as well as long-term memory (Levenson et al., 2004, J. Biol. Chem. 279:40545-40559; Lattal et al., 2007, Behav Neurosci 121:1125-1131; Vecsey et al., 2007, J. Neurosci 27:6128; Bredy, 2008, Learn Mem 15:460-467; Guan et al., 2009, Nature 459:55-60; Malvaez et al., 2010, Biol. Psychiatry 67:36-43; Roozendaal et al., 2010, J. Neurosci. 30:5037-5046). For example, HD AC inhibition can transform a learning event that does not lead to long-term memory into a learning event that does result in significant long-term memory (Stefanko et al., 2009, Proc. Natl. Acad. Sci. USA 106:9447-9452). Furthermore, HDAC inhibition can also generate a form of long-term memory that persists beyond the point at which normal memory fails. HDAC inhibitors have been shown to ameliorate cognitive deficits in genetic models of Alzheimer's disease (Fischer et al., 2007, Nature 447:178-182; Kilgore et al., 2010, Neuropsychopharmacology 35:870-880). These demonstrations suggest that modulating memory via HDAC inhibition has considerable therapeutic potential for many memory and cognitive disorders.

Currently, the role of individual HDACs in long-term memory has been explored in two recent studies. Kilgore et al. 2010, Neuropsychopharmacology 35:870-880 revealed that nonspecific HDAC inhibitors, such as sodium butyrate, inhibit class I HDACs (HDAC1, HDAC2, HDAC3, HDAC8) with little effect on the class IIa HDAC family members (HDAC4, HDAC5, HDAC7, HDAC9). This suggests that inhibition of class I HDACs may be critical for the enhancement of cognition observed in many studies. Indeed, forebrain and neuron specific over expression of HDAC2, but not HDAC1, decreased dendritic spine density, synaptic density, synaptic plasticity and memory formation (Guan et al., 2009, Nature, 459:55-60). In contrast, HDAC2 knockout mice exhibited increased synaptic density, increased synaptic plasticity and increased dendritic density in neurons. These HDAC2 deficient mice also exhibited enhanced learning and memory in a battery of learning behavioral paradigms. This work demonstrates that HDAC2 is a key regulator of synaptogenesis and synaptic plasticity. Additionally, Guan et al. showed that chronic treatment of mice with SAHA (an HDAC 1, 2, 3, 6, 8 inhibitor) reproduced the effects seen in the HDAC2 deficient mice and recused the cognitive impairment in the HDAC2 overexpression mice.

The inhibition of the HDAC2 (selectively or in combination with inhibition of other class I HDACs) is an attractive therapeutic target. Such inhibition has the potential for enhancing cognition and facilitating the learning process through increasing synaptic and dendritic density in neuronal cell populations. In addition, inhibition of HDAC2 may also be therapeutically useful in treating a wide variety of other diseases and disorders.

### SUMMARY

Provided herein are compounds of the Formula I:

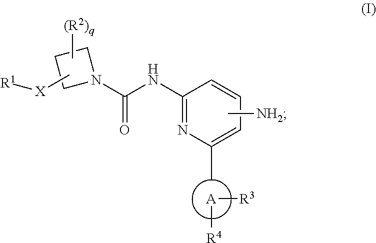

and pharmaceutically acceptable salts and compositions thereof, wherein X, R¹, R², R³, R⁴, q, and ring A are as described herein. The disclosed compounds and compositions modulate histone deacetylases (HDAC) (see e.g., Table 2 and 3), and are useful in a variety of therapeutic applications such as, for example, in treating neurological disorders, memory or cognitive function disorders or impairments, extinction learning disorders, fungal diseases or infections, inflammatory diseases, hematological diseases, neoplastic diseases, psychiatric disorders, and memory loss.

Certain compounds described herein have a substantial increase in inhibitory activity in cell lysate and recombinant enzymatic assays over homologous counterparts. For example, the introduction of a spacer group between the azetidinyl motif and R¹ (i.e., variable “X” in the compounds of Formula I) in certain compounds was found to result in a 100-fold increase in cell lysate potency, a greater than 7-fold increase in HDAC2 recombinant enzymatic assay inhibitory activity, and a 10-fold increase in HDAC1 recombinant enzymatic assay inhibitory activity when compared to the non-spacer containing analogue. Compare, for example, the activity differences between Compound 1 and Comparator A in Table 4. The only difference between the two compounds is the absence of variable X. Yet, a substantial increase in potency was realized from this modification. Similar trends were found for other compounds of Formula I. See e.g., Compound 6 and Comparator B, and Compound 14 and Comparator C in Table 4.

### DETAILED DESCRIPTION

### 1. General Description of Compounds

Provided herein is a compound of the Formula I:

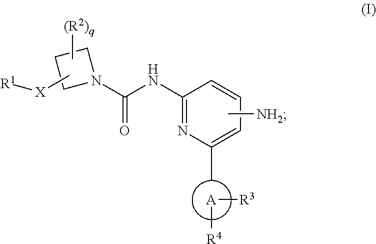

or a pharmaceutically acceptable salt thereof, wherein

ring A is phenyl or thiopheneyl;

X is (CR^{a}R^{b})ₜ, O, or NR⁵

q is 0, 1, or 2;

t is 1, 2, or 3;

R¹ is phenyl or heteroaryl, each of which are optionally substituted with 1 to 3 groups selected from R^{c};

R² is halo, (C₁-C₄)alkyl, (C₁-C₄)alkoxy, or OH;

R³ is hydrogen or halo;

R⁴ is halo when ring A is phenyl and R⁴ is hydrogen when ring A is thiopheneyl;

R⁵ is hydrogen, (C₁-C₄)alkyl, or (C₁-C₄)alkylO(C₁-C₄)alkyl;

R^{a} and R^{b} are each independently hydrogen, (C₁-C₄)alkyl, halo(C₁-C₄)alkyl, (C₁-C₄)alkoxy, or halo; and

R^{c} is halo, (C₁-C₄)alkyl, halo(C₁-C₄)alkyl, (C₁-C₄)alkoxy, halo(C₁-C₄)alkoxy, (C₁-C₄)alkylO(C₁-C₄)alkyl, (C₁-C₄)alkylNH(C₁-C₄)alkyl, (C₁-C₄)alkylN((C₁-C₄)alkyl)₂, —(C₁-C₄)alkylheteroaryl, or —(C₁-C₄)alkylheterocyclyl, wherein said heteroaryl and heterocyclyl are each optionally and independently substituted with 1 to 3 groups selected from (C₁-C₄)alkyl, halo(C₁-C₄)alkyl, (C₁-C₄)alkoxy, and halo.

### 2. Definitions

When used in connection to describe a chemical group that may have multiple points of attachment, a hyphen (-) designates the point of attachment of that group to the variable to which it is defined. For example, —(C₁-C₄)alkylheteroaryl and —(C₁-C₄)alkylheterocyclyl means that the point of attachment occurs on the (C₁-C₄)alkyl residue.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, means a saturated straight-chain or branched monovalent hydrocarbon radical. Unless otherwise specified, an alkyl group typically has 1-6 carbon atoms, i.e., (C₁-C₆)alkyl.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., but are not limited to —OCHF₂ or —OCF₃.

The term “heteroaryl” refers to a 5- to 12-membered (e.g., 5- or 6-membered) aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bicyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered (e.g., 4- to 6-membered) saturated or partially unsaturated heterocyclic ring containing 1 to 4 heteroatoms independently selected from N, O, and S. A heterocyclyl group can be mononcyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “fused” refers to two rings that share two adjacent ring atoms with one another.

The term “spiro” refers to two rings that shares one ring atom (e.g., carbon).

The term “bridged” refers to two rings that share three ring atoms with one another.

Enantiomers are one type of stereoisomer that can arise from a chiral center or chiral centers. Enantiomers are pairs of stereoisomers whose mirror images are not superimposable, most commonly because they contain an asymmetrically substituted carbon atom or carbon atoms that acts as a chiral center(s). “R” and “S” represent the absolute configuration of substituents around one or more chiral carbon atoms, where each chiral center is assigned the prefix “R” or “S” according to whether the chiral center configuration is right- (clockwise rotation) or left-handed (counter clockwise rotation). If the turn is clockwise or right-handed about a chiral carbon, the designation is “R” for rectus. If the turn is counter clockwise or left-handed about a chiral carbon, the designation is “S” for sinister.

When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a compound is depicted structurally without indicating the stereochemistry at a chiral center, the structure includes either configuration at the chiral center or, alternatively, any mixture of configurations at the chiral center stereoisomers.

“Racemate” or “racemic mixture” means a compound of equimolar quantities of two enantiomers, wherein such mixtures exhibit no optical activity, i.e., they do not rotate the plane of polarized light.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

Pharmaceutically acceptable salts as well as the neutral forms of the compounds described herein are included. For use in medicines, the salts of the compounds refer to non-toxic “pharmaceutically acceptable salts.” Pharmaceutically acceptable salt forms include pharmaceutically acceptable acidic/anionic or basic/cationic salts. Pharmaceutically acceptable basic/cationic salts include, the sodium, potassium, calcium, magnesium, diethanolamine, n-methyl-D-glucamine, L-lysine, L-arginine, ammonium, ethanolamine, piperazine and triethanolamine salts. Pharmaceutically acceptable acidic/anionic salts include, e.g., the acetate, benzenesulfonate, benzoate, bicarbonate, bitartrate, carbonate, citrate, dihydrochloride, gluconate, glutamate, glycollylarsanilate, hexylresorcinate, hydrobromide, hydrochloride, malate, maleate, malonate, mesylate, nitrate, salicylate, stearate, succinate, sulfate, tartrate, and tosylate.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence.

The term “effective amount” or “therapeutically effective amount” includes an amount of a compound described herein that will elicit a biological or medical response of a subject e.g., between 0.01-100 mg/kg body weight/day of the provided compound, such as e.g., 0.1-100 mg/kg body weight/day.

### 5. Description of Exemplary Compounds

In a first embodiment, provided herein is a compound of the Formula I:

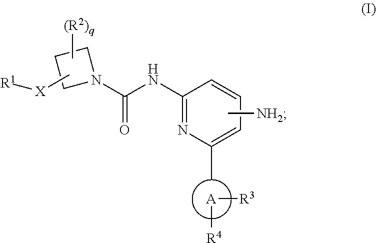

or a pharmaceutically acceptable salt thereof, wherein the variables are as described above for Formula I.

In a second embodiment, provided herein is a compound of the Formula II or IIa:

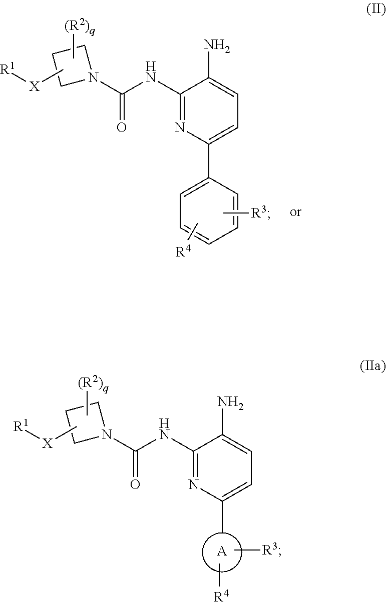

or a pharmaceutically acceptable salt thereof, wherein the variables are as described above for Formula I.

In a third embodiment, provided herein is a compound of the Formula III or IIIa:

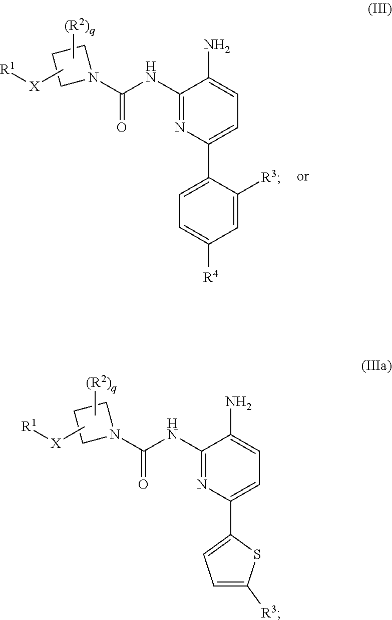

or a pharmaceutically acceptable salt thereof, wherein the variables are as described above for Formula I.

In a fourth embodiment, provided herein is a compound of the Formula IV or IVa.

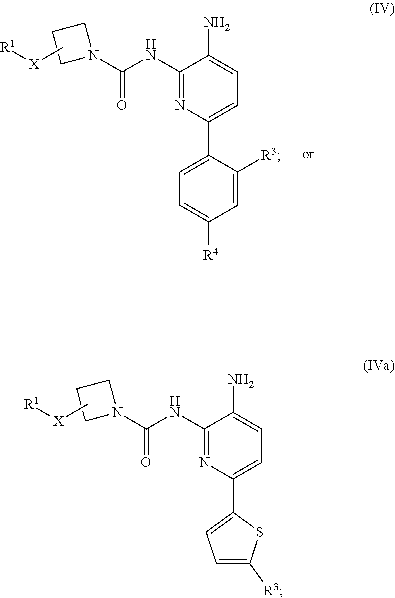

or a pharmaceutically acceptable salt thereof, wherein the variables are as described above for Formula I.

In a fifth embodiment, R³ in any one of Formula I, II, IIa, III, IIIa, IV, or IVa is halo, wherein the remaining variables are as described above for Formula I. Alternatively, R³ in any one of Formula I, II, IIa, III, IIIa, IV, or IVa is fluoro, wherein the remaining variables are as described above for Formula I. In another alternative, R³ in any one of Formula I, II, IIa, III, IIIa, IV, or IVa is hydrogen, wherein the remaining variables are as described above for Formula I.

In a sixth embodiment, R⁴ in any one of Formula I, II, IIa, III, IIIa, IV, or IVa is fluoro, wherein the remaining variables are as described above for Formula I, or the fifth embodiment.

In a seventh embodiment, X in any one of Formula I, II, IIa, III, IIIa, IV, or IVa is (CR^{a}R^{b})ₜ, wherein the remaining variables are as described above for Formula I, or the fifth or sixth embodiment.

In an eighth embodiment, R^{a} in any one of Formula I, II, IIa, III, IIIa, IV, or IVa is hydrogen, (C₁-C₄)alkyl, or halo; and R^{b} is hydrogen or halo, wherein the remaining variables are as described above for Formula I, or the fifth, sixth, or seventh embodiment. Alternatively, R^{a} in any one of Formula I, II, IIa, III, IIIa, IV, or IVa is hydrogen, methyl, or fluoro; and R^{b} is hydrogen or fluoro, wherein the remaining variables are as described above for Formula I, or the fifth, sixth, or seventh embodiment. In another alternative, R^{a} is hydrogen and R^{b} is halo (e.g., fluoro), wherein the remaining variables are as described above for Formula I, or the fifth, sixth, or seventh embodiment. In another alternative, R^{a} is halo (e.g., fluoro) and R^{b} is halo (e.g., fluoro), wherein the remaining variables are as described above for Formula I, or the fifth, sixth, or seventh embodiment.

In a ninth embodiment, t in any one of Formula I, II, IIa, III, IIIa, IV, or IVa is 1 or 2, wherein the remaining variables are as described above for Formula I, or the fifth, sixth, seventh, or eighth embodiment.

In a tenth embodiment, provided herein is a compound of the Formula V or Va:

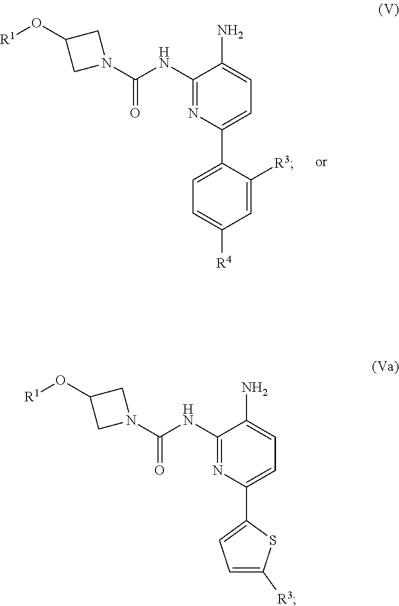

or a pharmaceutically acceptable salt thereof, wherein the variables are as described above for Formula I, or the fifth or sixth embodiment.

In an eleventh embodiment, provided herein is a compound of the Formula VI or VIa:

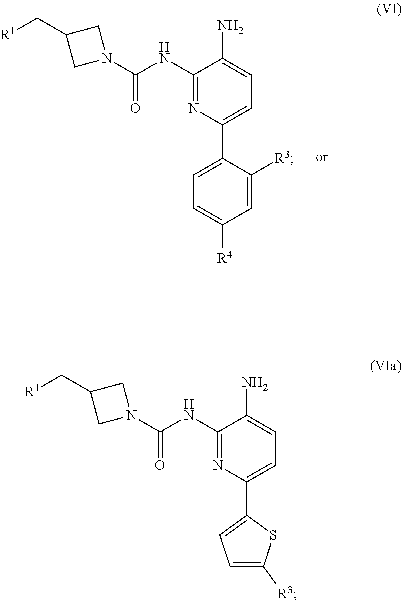

or a pharmaceutically acceptable salt thereof, wherein the variables are as described above for Formula I, or the fifth or sixth embodiment.

In a twelfth embodiment, R¹ in any one of Formula I, II, IIa, III, IIIa, IV, IVa, V, Va, VI, or VIa is heteroaryl optionally substituted with 1 to 2 groups selected from R^{c}, wherein the remaining variables are as described above for Formula I, or the fifth, sixth, seventh, eighth, or ninth embodiment. Alternatively, R¹ in any one of Formula I, II, IIa, III, IIIa, IV, IVa, V, Va, VI, or VIa is pyrimidinyl, pyridinyl, imidazopyridinyl, pyrazinyl, pyrazolyl, imidazolyl, oxazolyl, thiazolyl, or thiadiazolyl, each of which is optionally substituted with 1 to 2 groups selected from R^{c}, wherein the remaining variables are as described above for Formula I, or the fifth, sixth, seventh, eighth, or ninth embodiment.

In a thirteenth embodiment, R^{c} in any one of Formula I, II, IIa, III, IIIa, IV, IVa, V, Va, VI, or VIa is halo, halo(C₁-C₄)alkyl, (CᵣC₄)alkyl, or (C₁-C₄)alkylO(C₁-C₄)alkyl, wherein the remaining variables are as described above for Formula I, or the fifth, sixth, seventh, eighth, ninth, or twelfth embodiment. Alternatively, R^{c} in any one of Formula I, II, IIa, III, IIIa, IV, IVa, V, Va, VI, or VIa is fluoro, CF₃, methyl, or CH₂OCH₃, wherein the remaining variables are as described above for Formula I, or the fifth, sixth, seventh, eighth, ninth, or twelfth embodiment.

In a fourteenth embodiment, provided is a compound as described below in the Exemplification section. Pharmaceutically acceptable salts and free forms of the exemplified compounds are included.

### 4. Uses, Formulation and Administration

In some embodiments, the compounds and compositions described herein are useful in treating conditions associated with the activity of HD AC. Such conditions include for example, those described below.

Recent reports have detailed the importance of histone acetylation in central nervous system (“CNS”) functions such as neuronal differentiation, memory formation, drug addiction, and depression (Citrome, Psychopharmacol. Bull. 2003, 37, Suppl. 2, 74-88; Johannessen, CNS Drug Rev. 2003, 9, 199-216; Tsankova et al., 2006, Nat. Neurosci. 9, 519-525). Thus, in one aspect, the provided compounds and compositions may be useful in treating a neurological disorder. Examples of neurological disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, muscular dystrophy, olivopontocerebellar atrophy, multiple system atrophy, Wilson's disease, progressive supranuclear palsy, diffuse Lewy body disease, fronto-temporal lobar degeneration (FTLD), corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapie, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, restless leg syndrome, vertigo as result of CNS degeneration; pathologies arising with chronic alcohol or drug abuse including, for example, the degeneration of neurons in locus coeruleus and cerebellum, drug-induced movement disorders; pathologies arising with aging including degeneration of cerebellar neurons and cortical neurons leading to cognitive and motor impairments; and pathologies arising with chronic amphetamine abuse to including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor) and Wemicke-Korsakoff's related dementia. Neurological disorders affecting sensory neurons include Friedreich's ataxia, diabetes, peripheral neuropathy, and retinal neuronal degeneration. Other neurological disorders include nerve injury or trauma associated with spinal cord injury. Neurological disorders of limbic and cortical systems include cerebral amyloidosis, Pick's atrophy, and Rett syndrome. In another aspect, neurological disorders include disorders of mood, such as affective disorders and anxiety; disorders of social behavior, such as character defects and personality disorders; disorders of learning, memory, and intelligence, such as mental retardation and dementia. Thus, in one aspect the disclosed compounds and compositions may be useful in treating schizophrenia, delirium, attention deficit disorder (ADD), schizoaffective disorder, Alzheimer's disease, Rubinstein-Taybi syndrome, depression, mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder and disinhibition.

Transcription is thought to be a key step for long-term memory processes (Alberini, 2009, Physiol. Rev. 89, 121-145). Transcription is promoted by specific chromatin modifications, such as histone acetylation, which modulate histone-DNA interactions (Kouzarides, 2007, Cell, 128:693-705). Modifying enzymes, such as histone acetyltransferases (HATs) and histone deacetylases (HDACs), regulate the state of acetylation on histone tails. In general, histone acetylation promotes gene expression, whereas histone deacetylation leads to gene silencing. Numerous studies have shown that a potent HAT, cAMP response element-binding protein (CREB)-binding protein (CBP), is necessary for long-lasting forms of synaptic plasticity and long term memory (for review, see Barrett, 2008, Learn Mem 15:460-467). Thus, in one aspect, the provided compounds and compositions may be useful for promoting cognitive function and enhancing learning and memory formation.

The compounds and compositions described herein may also be used for treating fungal diseases or infections.

In another aspect, the compounds and compositions described herein may be used for treating inflammatory diseases such as stroke, rheumatoid arthritis, lupus erythematosus, ulcerative colitis and traumatic brain injuries (Leoni et al., PNAS, 99(5); 2995-3000 (2002); Suuronen et al. J. Neurochem. 87; 407-416 (2003) and Drug Discovery Today, 10: 197-204 (2005).

In yet another aspect, the compounds and compositions described herein may be used for treating a cancer caused by the proliferation of neoplastic cells. Such cancers include e.g., solid tumors, neoplasms, carcinomas, sarcomas, leukemias, lymphomas and the like. In one aspect, cancers that may be treated by the compounds and compositions described herein include, but are not limited to: cardiac cancer, lung cancer, gastrointestinal cancer, genitourinary tract cancer, liver cancer, nervous system cancer, gynecological cancer, hematologic cancer, skin cancer, and adrenal gland cancer. In one aspect, the compounds and compositions described herein are useful in treating cardiac cancers selected from sarcoma (angiosarcoma, fibrosarcoma, rhabdomyosarcoma, liposarcoma), myxoma, rhabdomyoma, fibroma, lipoma and teratoma. In another aspect, the compounds and compositions described herein are useful in treating a lung cancer selected from bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, and mesothelioma. In one aspect, the compounds and compositions described herein are useful in treating a gastrointestinal cancer selected from esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), and large bowel (adenocarcinoma, tubular adenoma, villous adenoma, hamartoma, leiomyoma). In one aspect, the compounds and compositions described herein are useful in treating a genitourinary tract cancer selected from kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma). In one aspect, the compounds and compositions described herein are useful in treating a liver cancer selected from hepatoma (hepatocellular carcinoma), cholangiocarcinoma, hepatoblastoma, angiosarcoma, hepatocellular adenoma, and hemangioma.

In some embodiments, the compounds described herein relate to treating, a bone cancer selected from osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors.

In one aspect, the compounds and compositions described herein are useful in treating a nervous system cancer selected from skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), and spinal cord (neurofibroma, meningioma, glioma, sarcoma).

In one aspect, the compounds and compositions described herein are useful in treating a gynecological cancer selected from uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

In one aspect, the compounds and compositions described herein are useful in treating a skin cancer selected from malignant melanoma, basal cell carcinoma, squamous cell carcinoma, Karposi's sarcoma, moles dysplastic nevi, lipoma, angioma, dermatofibroma, keloids, and psoriasis.

In one aspect, the compounds and compositions described herein are useful in treating an adrenal gland cancer selected from neuroblastoma.

In one aspect, the compounds and compositions described herein are useful in treating cancers that include, but are not limited to: leukemias including acute leukemias and chronic leukemias such as acute lymphocytic leukemia (ALL), Acute myeloid leukemia (AML), chronic lymphocytic leukemia (CLL), chronic myelogenous leukemia (CML) and Hairy Cell Leukemia; lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), Hodgkin's disease and non-Hodgkin's lymphomas, large-cell lymphomas, diffuse large B-cell lymphoma (DLBCL); Burkitt's lymphoma; mesothelioma, primary central nervous system (CNS) lymphoma; multiple myeloma; childhood solid tumors such as brain tumors, neuroblastoma, retinoblastoma, Wilm's tumor, bone tumors, and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer and thyroid cancer.

In one aspect, the compounds and compositions described herein are useful in treating a condition is selected from Alzheimer's disease, Huntington's disease, fronto-temporal lobar degeneration, Friedreich's ataxia, post-traumatic stress disorder, Parkinson's disease, Parkinson's disease dementia, substance dependence recovery, memory or cognitive function disorder or impairment, neurological disorder with synaptic pathology, disorder of learning distinction, psychiatric disorders, cognitive function or impairment associated with Alzheimer's disease, Lewy body dementia, schizophrenia, Rubinstein Taybi syndrome, Rett Syndrome, Fragile X, multiple sclerosis, age associated memory impairment, age related cognitive decline, and social, cognitive and learning disorders associated with autism.

In one aspect, provided herein is a method of treating a subject suffering from a neurological disorder, memory or cognitive function disorder or impairment, extinction learning disorder, fungal disease or infection, inflammatory disease, hematological disease, psychiatric disorders, and neoplastic disease, comprising administering to the subject an effective amount a compound described herein, or a pharmaceutically acceptable salt thereof, or the composition comprising a compound described herein.

Also provided herein is a method of treating a subject suffering from (a) a cognitive function disorder or impairment associated with Alzheimer's disease, posterior cortical atrophy, normal-pressure hydrocephalus, Huntington's disease, seizure induced memory loss, schizophrenia, Rubinstein Taybi syndrome, Rett Syndrome, depression, Fragile X, Lewy body dementia, vascular dementia, vascular cognitive impairment (VCI), Binswanger's Disease, fronto-temporal lobar degeneration (FTLD), ADHD, dyslexia, major depressive disorder, bipolar disorder and social, cognitive and learning disorders associated with autism, traumatic brain injury (TBI), chronic traumatic encephalopathy (CTE), multiple sclerosis (MS), attention deficit disorder, anxiety disorder, conditioned fear response, panic disorder, obsessive compulsive disorder, posttraumatic stress disorder (PTSD), phobia, social anxiety disorder, substance dependence recovery, Age Associated Memory Impairment (AAMI), Age Related Cognitive Decline (ARCD), ataxia, Parkinson's disease, or Parkinson's disease dementia; or (b) a hematological disease selected from acute myeloid leukemia, acute promyelocytic leukemia, acute lymphoblastic leukemia, chronic myelogenous leukemia, myelodysplastic syndromes, and sickle cell anemia; or (c) a neoplastic disease; or (d) a disorder of learning extinction selected from fear extinction and post-traumatic stress disorder; or (e) hearing loss or a hearing disorder; or (f) fibrotic diseases, such as pulmonary fibrosis, renal fibrosis, cardiac fibrosis, and scleroderma; or (g) bone pain in patients with cancer; or (h) neuropathic pain; comprising administering to the subject an effective amount a compound described herein, or a pharmaceutically acceptable salt thereof, or the composition comprising a compound described herein.

Also provided is a method of treating a subject suffering from Alzheimer's disease, Huntington's disease, frontotemporal dementia, Friedreich's ataxia, post-traumatic stress disorder (PTSD), Parkinson's disease, or substance dependence recovery, comprising administering to the subject an effective amount a compound described herein, or a pharmaceutically acceptable salt thereof, or the composition comprising a compound described herein.

Also provided is a compound described herein, or a pharmaceutically acceptable salt thereof, or a provided composition, for treating one or more of the disclosed conditions.

Also provided is a compound described herein, or a pharmaceutically acceptable salt thereof, or a provided composition, for the manufacture of a medicament for treating one or more of the disclosed conditions.

Subjects may also be selected to be suffering from one or more of the described conditions prior to treatment with a compound described herein, or a pharmaceutically acceptable salt thereof, or a provided composition.

The present disclosure also provides pharmaceutically acceptable compositions comprising a compound described herein, or a pharmaceutically acceptable salt thereof; and a pharmaceutically acceptable carrier. These compositions can be used to treat one or more of the conditions described above.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Liquid dosage forms, injectable preparations, solid dispersion forms, and dosage forms for topical or transdermal administration of a compound are included herein.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided compound in the composition will also depend upon the particular compound in the composition.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### General Information

Spots were visualized by UV light (254 and 365 nm). Purification by column and flash chromatography was carried out using silica gel (200-300 mesh). Solvent systems are reported as the ratio of solvents.

NMR spectra were recorded on a Bruker 400 (400 MHz) spectrometer. ¹H chemical shifts are reported in 8 values in ppm with tetramethylsilane (TMS, =0.00 ppm) as the internal standard. See, e.g., the data provided in Table 1.

LCMS spectra were obtained on an Agilent 1200 series 6110 or 6120 mass spectrometer with ESI (+) ionization mode. See, e.g., the data provided in Table 1.

### Example 1

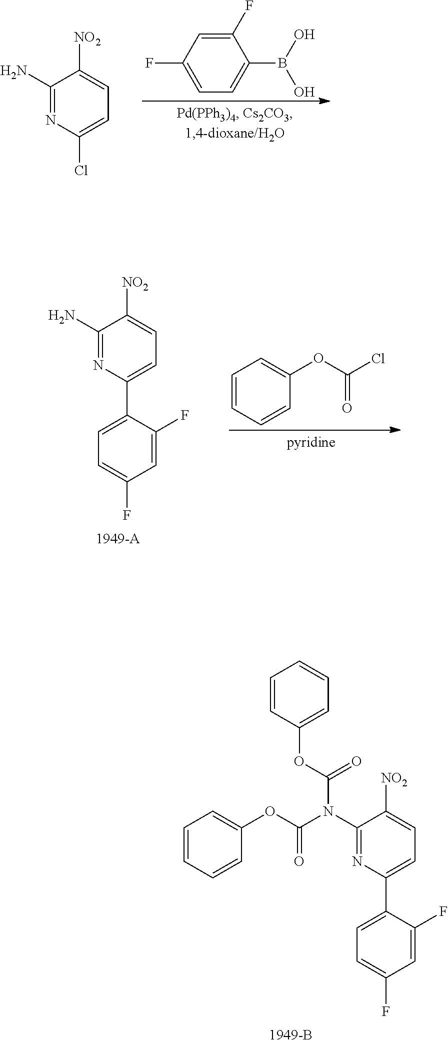 [SMILES: Nc1nc(Cl)ccc1[N+](=O)[O-] ; OB(O)c1ccc(F)cc1F ; Nc1nc(-c2ccc(F)cc2F)ccc1[N+](=O)[O-] ; O=C(Cl)Oc1ccccc1 ; O=C(Oc1ccccc1)N(C(=O)Oc1ccccc1)c1nc(-c2ccc(F)cc2F)ccc1[N+](=O)[O-]]

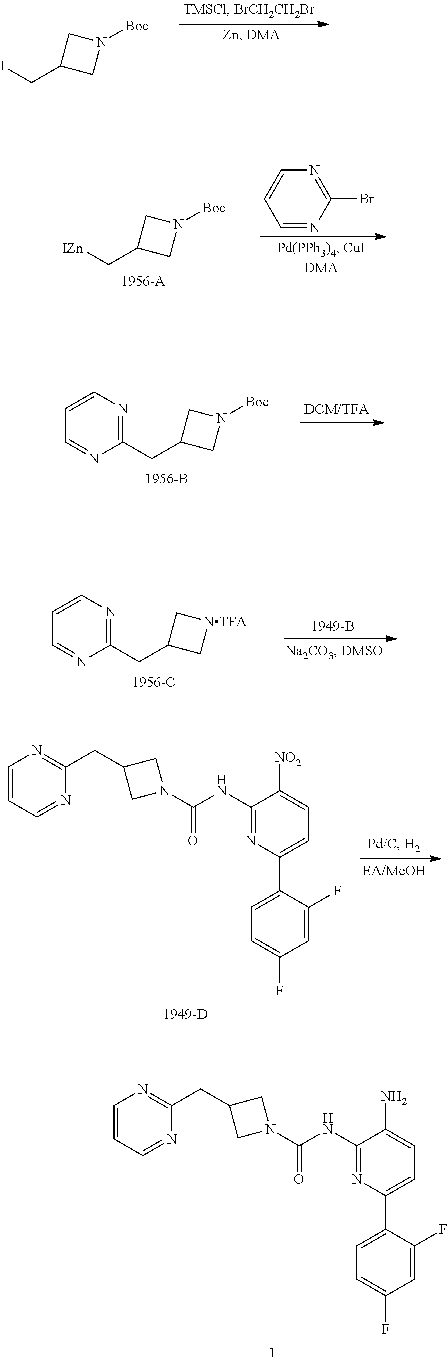

Synthesis of 1949-A. A mixture of 6-chloro-3-nitropyridin-2-amine (4.58 g, 26.4 mmol), 2,4-difluorophenylboronic acid (5.00 g, 31.7 mmol) and Cs₂CO₃ (25.73 g, 79.2 mmol) in dioxane/H₂O (100 mL/10 mL) was treated with Pd(PPh₃)₄ (1.10 g, 0.95 mmol) under a N₂ atmosphere. The mixture was stirred at 100° C. for 2 h and then concentrated in vacuo. The residue was dissolved with EtOAc (200 mL) and the resulting solution was washed with brine (100 mL×3). The organic layer was dried over anhydrous Na₂SO₄ and then concentrated in vacuo. The residue was purified by column chromatography on silica gel (PE:EtOAc=7:1˜5:1) to give 1949-A (4.0 g, 61%) as a yellow solid. MS 252.1 [M+H]⁺.

Synthesis of 1949-B. A stirred solution of 1949-A (4.0 g, 15.94 mmol) in pyridine (60 mL) was treated with phenyl carbonochloridate (7.50 g, 47.81 mmol) dropwise at 0° C. After the addition was completed, the mixture was stirred at 50° C. for 4 h. The mixture was then concentrated in vacuo, and the residue was purified by column chromatography on silica gel (PE:DCM=3:2˜1:1) to give 1949-B (7.1 g, 91%) as a yellow solid. MS 492.1 [M+H]⁺.

Synthesis of 1956-A. To a mixture of zinc dust (896 mg, 13.8 mmol) in anhydrous DMA (3 mL) was added TMSCl and 1,2-dibromoethane (0.24 mL, v/v=7/5), and the mixture was stirred at room temperature for 20 min under a N₂ atmosphere. A solution of tert-butyl 3-(iodomethyl)azetidine-1-carboxylate (3.15 g, 10.6 mmol) in anhydrous DMA (4 mL) was then added to the above mixture, and the resulting mixture was stirred at room temperature for 16 h under a N₂ atmosphere. The reaction mixture was used in the next step directly as 1956-A. The concentration of 1956-A was about 1.0 mol/L in DMA.

Synthesis of 1956-B. A mixture of 2-bromopyrimidine (265 mg, 1.67 mmol), CuI (32 mg, 0.17 mmol) and Pd(PPh₃)₄ (96 mg, 0.084 mmol) in anhydrous DMA (6 mL) under a N₂ atmosphere was treated with 1956-A (2.0 mL). The resulting mixture was stirred at 60° C. for 48 h under a N₂ atmosphere. The mixture was then diluted with water (30 mL) and extracted with EtOAc (20 mL×3). The combined organic layers were washed with brine (20 mL×3), dried over anhydrous Na₂SO₄ and then concentrated in vacuo. The residue was purified by Prep-TLC (EtOAc:PE=1:1) to give 1956-B (160 mg, 38%) as a yellow solid. MS 250.2 [M+H]⁺.

Synthesis of 1956-C. To a solution of 1956-B (160 mg, 0.64 mmol) in DCM (6 mL) was added TFA (2 mL) dropwise. Then the solution was stirred at room temperature for 1 h. The solution was concentrated in vacuo to give 1956-C as a crude product which was used in the next step directly without further purification. MS 150.2 [M+H]⁺.

Synthesis of 1956-D. A mixture of 1956-C (0.64 mmol, crude product from last step) and 1949-B (177 mg, 0.36 mmol) in DMSO (6 mL) was stirred at room temperature for 10 min, then Na₂CO₃ (377 mg, 3.55 mmol) was added into the above mixture and stirring was continued at room temperature for 2 h. The mixture was then diluted with water (30 mL) and extracted with EtOAc (10 mL×3). The combined organic layers were washed with brine (10 mL×3), dried over anhydrous Na₂SO₄ and then concentrated in vacuo. The residue was purified by Prep-TLC (DCM:EtOAc=1:1) to give 1956-D (70 mg, 46%) as a yellow solid. MS 427.2 [M+H]⁺.

Synthesis of Compound 1. A mixture of 1956-D (70 mg, 0.16 mmol) and Pd/C (70 mg) in MeOH/EtOAc (2 mL/2 mL) was stirred at room temperature for 50 min under a H₂ atmosphere. The Pd/C was removed by filtration through Celite, the filtrate was concentrated in vacuo, and the residue was purified by Prep-TLC (DCM:MeOH=30:1) to give 1 (40 mg, 63%) as a brown solid. MS 397.2 [M+H]⁺.

Compounds 2-27, 48, 49, and 50 were synthesized in a similar manner using appropriately substituted boronic acid and aryl bromide variants of reagents used to synthesize 1.

Compound 2. 15 mg, 36%, a yellow solid.

Compound 3. 100 mg, 57%, a white solid.

Compound 4. 20 mg, 21%, a yellow solid.

Compound 5. 20 mg, 42%, an off-white solid.

Compound 6. 50 mg, 72%, an off-white solid.

Compound 7. 35 mg, 63%, a light yellow solid.

Compound 8. 35 mg, 42%, a gray solid.

Compound 9. 15 mg, 40%, a orange solid.

Compound 10. 118 mg, 70%, a light yellow solid.

Compound 11. 90 mg, 48%, a yellow solid.

Compound 12. 40 mg, 29%, a light yellow solid.

Compound 13. 30 mg, 40%, a yellow solid.

Compound 14. 120 mg, 80%, a yellow solid.

Compound 15. 120 mg, 54%, a flesh color solid.

Compound 16.5 mg, 27%, a white solid.

Compound 17. 90 mg, 53%, a white solid.

Compound 18. 85 mg, 53%, a white solid.

Compound 19. 80 mg, 43%, a white solid.

Compound 20. 10 mg, 36%, a orange solid.

Compound 21. 60 mg, 58%, a light yellow solid.

Compound 22. 90 mg, 54%, a yellow solid.

Compound 23. 100 mg, 43%, a yellow solid.

Compound 24. 28 mg, 32%, a light yellow solid.

Compound 25. 55 mg, 59%, a white solid.

Compound 26. 20 mg, 43%, an off-white solid.

Compound 27. 25 mg, 58%, a light yellow solid.

Compound 48. 15 mg, 36%, a yellow solid.

Compound 49. 100 mg, 57%, a white solid.

Compound 50. 53 mg, 44%, an off-white solid.

### Example 2

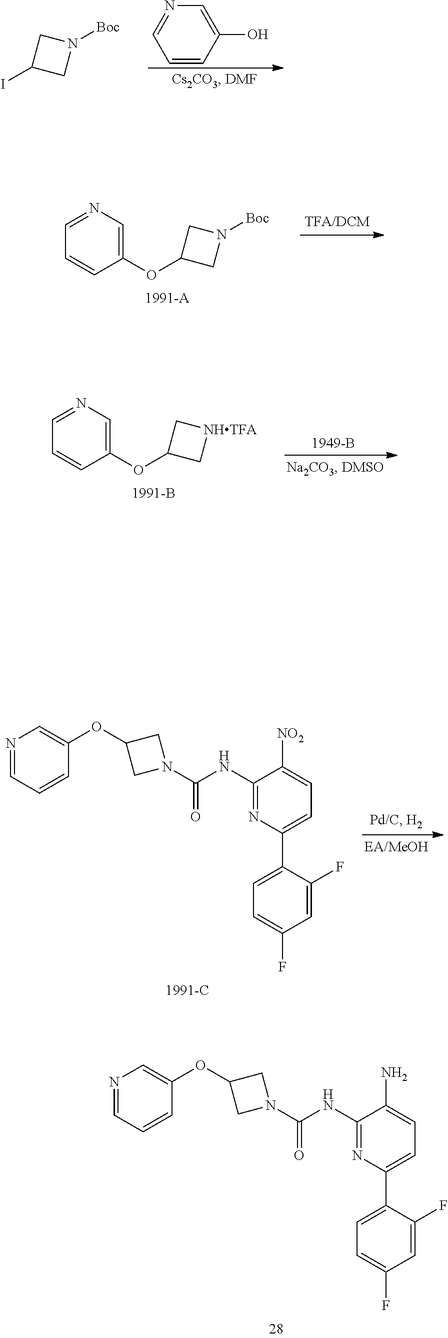

Synthesis of 1991-A. A mixture of tert-butyl 3-iodoazetidine-1-carboxylate (600 mg, 2.12 mmol), pyridin-3-ol (168 mg, 1.77 mmol) and Cs₂CO₃ (865 mg, 2.66 mol) in DMF (10 mL) was stirred at 100° C. for 3 h. The mixture was diluted with water (30 mL) and extracted with EtOAc (10 mL×3). The combined organic layers were washed with brine (10 mL×3), dried over anhydrous Na₂SO₄ and then concentrated in vacuo. The residue was purified by column chromatography on silica gel (DCM:EtOAc=10:1˜1:1) to give 1991-A (300 mg, 68%) as a white solid. MS 195.3 [M-56+H]⁺.

Synthesis of 1991-B. To a solution of 1991-A (300 mg, 1.20 mmol) in DCM (6 mL) was added TFA (2 mL) dropwise. Then the solution was stirred at room temperature for 1 h. The solution was concentrated in vacuo to give 1991-B as a crude product which was used in the next step directly without further purification. MS 151.3 [M+H]⁺.

Synthesis of 199NC. A mixture of 1991-B (1.20 mmol, crude product from last step) and 1949-B (329 mg, 0.67 mmol) in DMSO (10 mL) was stirred at room temperature for 10 min, then Na₂CO₃ (707 mg, 6.67 mmol) was added into the above mixture and stirring was continued at room temperature for 2 h. The mixture was then diluted with water (30 mL) and extracted with EtOAc (20 mL×3). The combined organic layers were washed with brine (20 mL×3), dried over anhydrous Na₂SO₄ and then concentrated in vacuo. The residue was purified by Prep-TLC (DCM:MeOH=30:1) to give 1991-C (160 mg, 56%) as a yellow solid. MS 428.1 [M+H]⁺.

Synthesis of Compound 28. A mixture of 1991-D (160 mg, 0.37 mmol) and Pd/C (160 mg) in MeOH/EtOAc (5 mL/5 mL) was stirred at room temperature for 50 min under a H₂ atmosphere. The Pd/C was removed by filtration through Celite, the filtrate was concentrated in vacuo, and the residue was purified by Prep-TLC (DCM:MeOH=20:1) to give 28 (80 mg, 54%) as a light yellow solid. MS 199.6 [M/2+H]⁺, 398.1 [M+H]⁺, 420.1 [M+23]⁺.

Compound 29 was synthesized in a similar manner using an appropriately substituted alcohol variant of reagents used to synthesize 28.

Compound 29. 40 mg, 21%, a white solid.

### Example 3

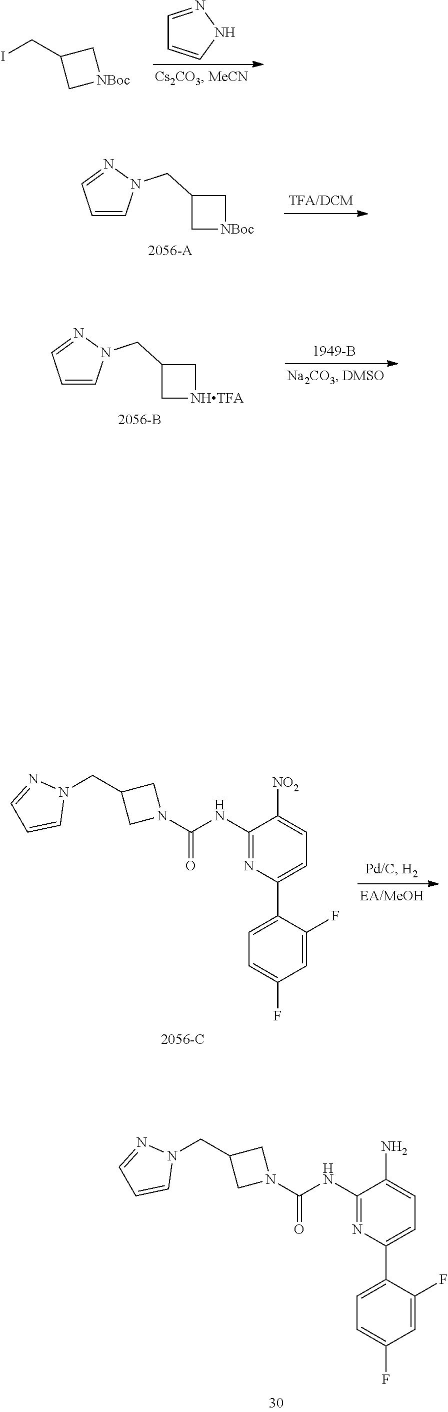

Synthesis of 2056-A. A mixture of tert-butyl 3-(iodomethyl)azetidine-1-carboxylate (419 mg, 1.41 mmol), pyrazole (80 mg, 1.18 mmol) and Cs₂CO₃ (769 mg, 2.36 mol) in acetonitrile (10 mL) was stirred at 80° C. for 3 h. The mixture was diluted with water (30 mL) and extracted with EtOAc (10 mL×3). The combined organic layer was washed with brine (10 mL×3), dried over anhydrous Na₂SO₄ and then concentrated in vacuo. The residue was purified by column chromatography on silica gel (PE:EtOAc=10:1˜1:1) to give 2056-A (190 mg, 68%) as a white solid. MS 238.3 [M+H]⁺.

Synthesis of 2056-B. To a solution of 2056-A (190 mg, 0.80 mmol) in DCM (6 mL) was added TFA (2 mL) dropwise. Then the solution was stirred at room temperature for 1 h. The solution was concentrated in vacuo to give 2056-B as a crude product used to next step directly. MS 138.3 [M+H]⁺.

Synthesis of 2056-C. A mixture of 2056-B (0.80 mmol, crude product from last step) and 1949-B (218 mg, 0.44 mmol) in DMSO (6 mL) was stirred at room temperature for 10 min, then Na₂CO₃ (471 mg, 4.44 mmol) was added into above mixture and stirred at room temperature for 2 h. The mixture was diluted with water (20 mL) and extracted with EtOAc (10 mL×3). The combined organic layer was washed with brine (10 mL×3), dried over anhydrous Na₂SO₄ and then concentrated in vacuo. The residue was purified by Prep-TLC (DCM:MeOH=40:1) to give 2056-C (120 mg, 66%) as a yellow solid. MS 428.1 [M+H]⁺.

Synthesis of Compound 30. A mixture of 2056-C (120 mg, 0.29 mmol) and Pd/C (120 mg) in MeOH/EtOAc (5 mL/5 mL) was stirred at room temperature for 50 min under H₂ atmosphere. Pd/C was removed by filtration through Celite. The filtrate was concentrated in vacuo and the residue was purified by Prep-TLC (DCM:MeOH=20:1) to give 30 (68 mg, 61%) as a white solid. MS 385.2 [M+H]⁺.

Compound 31 was synthesized in a similar manner using imidazole.

Compound 31 85 mg, 83%, a white solid.

### Example 4

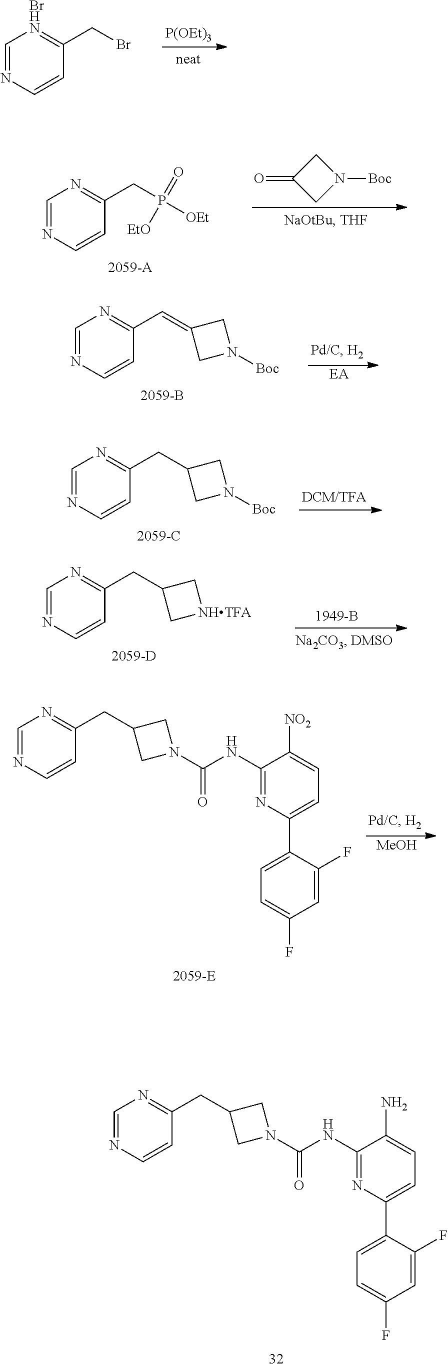

Synthesis of 2059-A. A solution of 4-(bromomethyl)pyrimidine hydrobromide (450 mg, 1.77 mmol) in P(OEt)₃ (10 mL) was stirred at 160° C. for 4 h. The mixture was then concentrated in vacuo and the residue was purified by column chromatography on silica gel (PE:EtOAc=10:1 to EtOAc) to give 2059-A (220 mg, 54%) as a yellow solid. MS 231.2 [M+H]⁺.

Synthesis of 2059-B. To a solution of 2059-A (220 mg, 0.96 mmol) in THF (10 mL) was added tert-butyl 3-oxoazetidine-1-carboxylate (213 mg, 1.3 mmol) and tBuONa (240 mg, 2.5 mmol) at room temperature. The resulting solution was stirred at room temperature for 3 h, then the mixture was diluted with water (20 ml), and extracted with EtOAc (30 mL×3). The combined organic layers were washed with brine (30 mL×3), dried over anhydrous Na₂SO₄ and then concentrated? in vacuo. The residue was purified by column chromatography on silica gel (PE:EtOAc=10:1 to EtOAc) to give 2059-B (100 mg, 42%) as a light yellow solid. MS 248.2 [M+H]⁺.

Synthesis of 2059-C. A mixture of 2059-B (100 mg, 0.41 mmol) and Pd/C (100 mg) in EtOAc (6 mL) was stirred at room temperature for 1 h under a H₂ atmosphere. The Pd/C was then removed by filtration through the Celite, the filtrate was concentrated, and the residue was purified by Prep-TLC (EtOAc:PE=10:1) to give 2059-C (90 mg, 89%) as a yellow solid. MS 250.2 [M+H]⁺.

Synthesis of 2059-D. To a solution of 2059-C (90 mg, 0.36 mmol) in DCM (6 mL) was added TFA (2 mL) dropwise. The resulting solution was stirred at room temperature for 1 h, whereupon the solvent was removed in vacuo to give 2059-D as a crude product which was used in the next step directly without further purification.

Synthesis of 2059-E. A mixture of 1949-B (98 mg, 0.2 mmol) and 2059-D (0.36 mmol, crude product from last step) in DMSO (5 mL) was treated with Na₂CO₃ (382 mg, 3.6 mmol) and the reaction mixture was stirred at room temperature for 2 h. The mixture was then diluted with water (20 mL) and extracted with EtOAc (20 mL×3). The combined organic layers were washed with brine (20 mL×3), dried over anhydrous Na₂SO₄ and then concentrated in vacuo. The residue was purified by Prep-TLC (EtOAc:PE=5:1) to give 2059-E (80.0 mg, 94%) as a yellow solid. MS 427.2 [M+H]⁺.

Synthesis of Compound 32. A mixture of 2059-E (80.0 mg, 0.188 mmol) and Pd/C (80.0 mg) in MeOH (6 mL) was stirred at room temperature for 1 h under a H₂ atmosphere. The Pd/C was then removed by filtration through the Celite, the filtrate was concentrated, and the residue was purified by Prep-TLC (EtOAc:MeOH=5:1) to give 32 (41 mg, 50%) as a white solid. MS 397.2 [M+H]⁺.

### Example 5

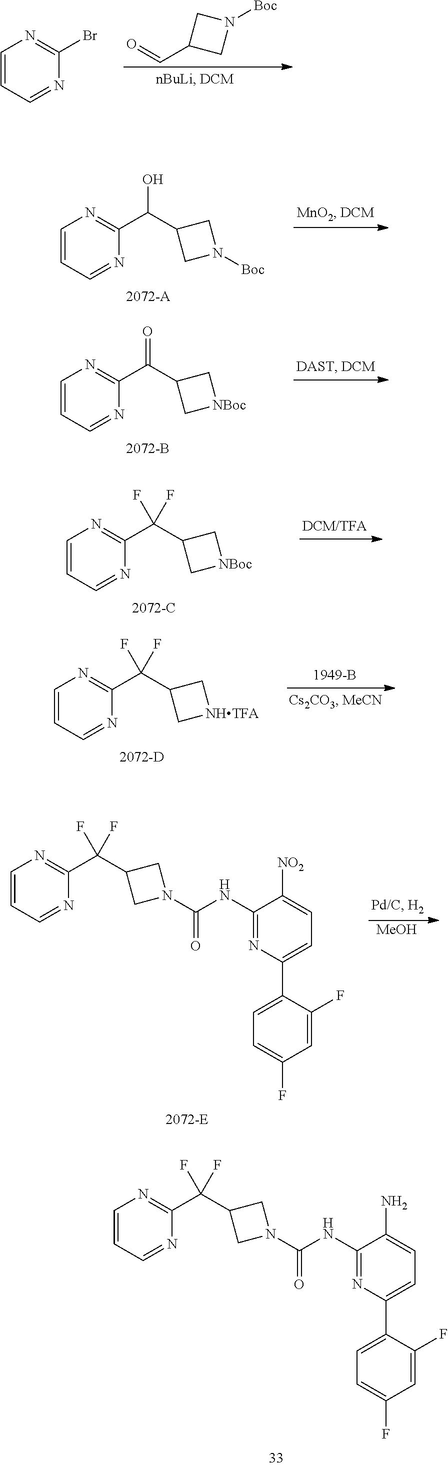

Synthesis of 2072-A. To a solution of 2-bromopyrimidine (1.0 g, 6.29 mmol) in DCM (20 mL) under a N₂ atmosphere was added nBuLi (3.0 mL, 7.55 mmol) dropwise at −78° C., and the reaction mixture was stirred at −78° C. for 1 h. Tert-butyl 3-formylazetidine-1-carboxylate (1.4 g, 7.55 mmol) in DCM (10 mL) was then added into above mixture dropwise at −78° C. The resulting mixture was then allowed to warm to room temperature and stirred at room temperature for 3 h. The mixture was then diluted with saturated aqueous NH₄Cl (40 mL), and extracted with DCM (20 mL×3). The combined organic layers were washed with brine (20 mL×3), dried over anhydrous Na₂SO₄ and then concentrated in vacuo. The residue was purified by column chromatography on silica gel (PE:EtOAc=10:1 to EtOAc) to give 2072-A (300 mg, 18%) as a light yellow solid. MS 266.2 [M+H]⁺.

Synthesis of 2072-B. A mixture of 2072-A (300 mg, 1.13 mmol) and MnO₂ (3.0 g) in DCM (20 mL) was stirred at room temperature for 4 h. The MnO₂ was then removed by filtration through the Celite. The filtrate was concentrated and the residue was purified by Prep-TLC (EtOAc:PE=10:1) to give 2072-B (150 mg, 50%) as a light yellow solid. MS 264.2 [M+H]⁺.

Synthesis of 2072-C. A solution of 2072-B (150 mg, 0.57 mmol) in DCM (10 mL) was treated with DAST (0.3 mL) dropwise at −78° C., and the reaction mixture was allowed to warm temperature, and then stirred at room temperature for 16 h. The solvent was then removed under reduced pressure and the residue was purified by Prep-TLC (EtOAc:PE=10:1) to give 2072-C (60 mg, 37%) as a brown solid. MS 286.2 [M+H]⁺.

Synthesis of 2072-D. To a solution of 2072-C (60 mg, 0.21 mmol) in DCM (3 mL) was added TFA (1 mL) dropwise. The resulting solution was stirred at room temperature for 1 h, whereupon the solvent was removed in vacuo to give 2072-D as a crude product which was used directly in the next step without further purification.

Synthesis of 2072-E. A mixture of 1949-B (86 mg, 0.18 mmol) and 2072-D (0.21 mmol, crude product from last step) in acetonitrile (10 mL) was treated with Cs₂CO₃ (285 mg, 0.88 mmol), and the reaction mixture was stirred at room temperature for 2 h. The mixture was then diluted with water (20 mL), and extracted with EtOAc (20 mL×3). The combined organic layers were washed with brine (20 mL×3), dried over anhydrous Na₂SO₄ and then concentrated in vacuo. The residue was purified by Prep-TLC (EtOAc:PE=5:1) to give 2072-E (60 mg, 74%) as a yellow solid. MS 463.2 [M+H]⁺.

Synthesis of Compound 33 A mixture of 2072-E (60 mg, 0.13 mmol) and Pd/C (60 mg) in MeOH (5 mL) was stirred at room temperature for 1 h under a H₂ atmosphere. The Pd/C was then removed by filtration through the Celite, the filtrate was concentrated and the residue was purified by Prep-TLC (EtOAc:MeOH=15:1) to give 33 (30 mg, 53%) as a light yellow solid. MS 433.2 [M+H]⁺.

Compounds 34, 35, 36, 37 and 38 were synthesized in a similar manner using appropriately substituted boronic acid and bromine variants of reagents used to synthesize 33.

Compound 34. 38 mg, 56%, an off-white solid.

Compound 35 15 mg, 26%, a white solid.

Compound 36. 17 mg, 37%, a light yellow solid.

Compound 37. 38 mg, 59%, a white solid.

Compound 38. 34 mg, 52%, a light yellow solid.

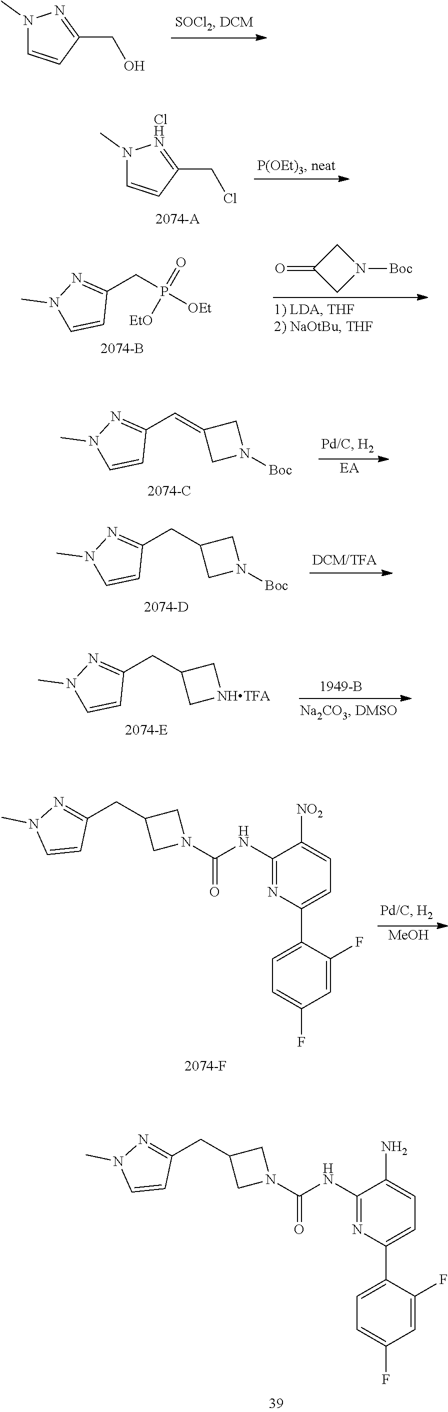

### Example 6

Synthesis of 2074-A To a solution of (1-methyl-1H-pyrazol-3-yl)methanol (1.0 g, 8.92 mmol) in DCM (10 mL) was added SOCl₂ (2.66 g, 22.3 mmol) dropwise at 0° C. The reaction mixture was then allowed to warm to room temperature, and was stirred at room temperature for 2 h. The mixture was then concentrated in vacuo to give 2074-A (1.0 g, 67%) as a white solid. MS 131.2 [M+H]⁺, MS 133.2 [M+H]⁺.

Synthesis of 2074-A, A solution of 2074-A (1.0 g, 6.0 mmol) in P(OEt)₃ (10 mL) was stirred at 145° C. for 16 h. The mixture was then concentrated in vacuo, and the residue was purified by column chromatography on silica gel (EtOAc to EtOAc:MeOH=10:1) to give 2074-B (550 mg, 40%) as a colorless oil. MS 233.2 [M+H]⁺.

Synthesis of 2074-C A solution of 2074-B (400 mg, 1.72 mmol) in THF (10 mL) was treated with a solution of LDA (2.6 mL, 5.2 mmol, 2 M in THF) dropwise at −78° C., and the reaction mixture was stirred for 1 h. A solution of tert-butyl 3-oxoazetidine-1-carboxylate (441 mg, 2.58 mmol) in THF (5 mL) was then added to the reaction mixture dropwise at −78° C., and the reaction was then allowed to warm to room temperature and stirred for 2 h. Finally, tBuONa (330 mg, 3.44 mmol) was added at room temperature and stirred was continued for another 4 h. The mixture was then diluted with saturated aqueous NH₄Cl (40 mL), and extracted with EtOAc (30 mL×3). The combined organic layers were washed with brine (30 mL×3), dried over anhydrous Na₂SO₄ and then concentrated in vacuo. The residue was purified by Prep-TLC (EtOAc) to give 2074-C (90 mg, 21%) as a white solid. MS 250.2 [M+H]⁺.

Synthesis of 2074-D. A mixture of 2074-C (90 mg, 0.36 mmol) and Pd/C (90 mg) in EtOAc (3 mL) was stirred at room temperature for 1 h under a H₂ atmosphere. The Pd/C was then removed by filtration through the Celite, the filtrate was concentrated, and the residue was purified by Prep-TLC (EtOAc) to give 2074-D (65 mg, 72%) as yellow solid. MS 252.2 [M+H]⁺.

Synthesis of 2074-E. To a solution of 2074-D (65 mg, 0.26 mmol) in DCM (3 mL) was added TFA (1 mL) dropwise. Then resulting solution was stirred at room temperature for 1 h, whereupon the solvent was removed in vacuo to give 2074-E as a crude product which was used directly in the next step without further purification.

Synthesis of 2074-F. A mixture of 1949-B (71 mg, 0.14 mmol) and 2059-D (0.26 mmol, crude product from last step) in DMSO (5 mL) was treated with Na₂CO₃ (153 mg, 1.44 mmol) and the reaction mixture was stirred at room temperature for 2 h. The mixture was then diluted with water (10 mL), and extracted with EtOAc (10 mL×3). The combined organic layers were washed with brine (10 mL×3), dried over anhydrous Na₂SO₄ and then concentrated in vacuo. The residue was purified by Prep-TLC (EtOAc:MeOH=50:1) to give 2074-F (50 mg, 83%) as a yellow solid. MS 429.2 [M+H]⁺.

Synthesis of Compound 39. A mixture of 2074-F (50 mg, 0.12 mmol) and Pd/C (50 mg) in MeOH (3 mL) was stirred at room temperature for 1 h under a H₂ atmosphere. The Pd/C was then removed by filtration through the Celite, the filtrate was concentrated, and the residue was purified by Prep-TLC (DCM:MeOH=30:1) to give 39 (30 mg, 63%) as a white solid. MS 399.2 [M+H]⁺.

### Example 7

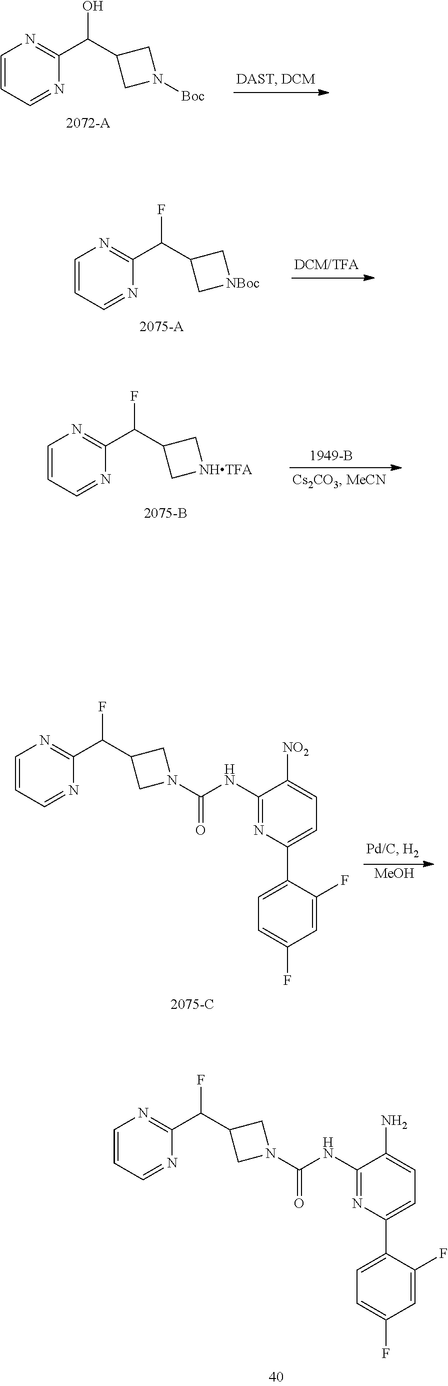

Synthesis of 2075-A. A solution of 2072-A (240 mg, 0.91 mmol) in DCM (10 mL) was treated with DAST (0.6 mL) dropwise at −78° C., and the reaction mixture was then allowed to warm to room temperature and stirred at room temperature for 16 h. The solvent was removed under reduced pressure and the residue was purified by Prep-TLC (EtOAc:PE=10:1) to give 2075-A (50 mg, 20%) as a brown solid. MS 268.2 [M+H]⁺.

Synthesis of 2075-B. To a solution of 2075-A (50 mg, 0.19 mmol) in DCM (3 mL) was added TFA (1 mL) dropwise. The resulting reaction mixture was stirred at room temperature for 1 h, whereupon the solvent was removed in vacuo to give 2075-B as a crude product which was used directly in the next step without further purification.

Synthesis of 2075-C. A mixture of 1949-B (78 mg, 0.16 mmol) and 2075-B (0.19 mmol, crude product from last step) in acetonitrile (10 mL) was treated with Cs₂CO₃ (247 mg, 0.76 mmol), and the reaction mixture was stirred at room temperature for 2 h. The mixture was then diluted with water (20 mL), and extracted with EtOAc (20 mL×3). The combined organic layers were washed with brine (20 mL×3), dried over anhydrous Na₂SO₄ and then concentrated in vacuo. The residue was purified by Prep-TLC (EtOAc:PE=5:1) to give 2075-C (50 mg, 70%) as a yellow solid. MS 445.0 [M+H]⁺.

Synthesis of Compound 40 A mixture of 2075-C (50 mg, 0.11 mmol) and Pd/C (50 mg) in MeOH (4 mL) was stirred at room temperature for 1 h under a H₂ atmosphere. The Pd/C was then removed by filtration through Celite, the filtrate was concentrated in vacuo, and the residue was purified by Prep-TLC (EA:MeOH=15:1) to give 40 (17.0 mg, 37%) as a white solid. MS 415.2 [M+H]⁺.

Compound 41 was synthesized in a similar manner using an appropriately substituted bromine variant of reagents used to synthesize 40.

Compound 41. 20 mg, 31%, a light yellow solid.

### Example 8

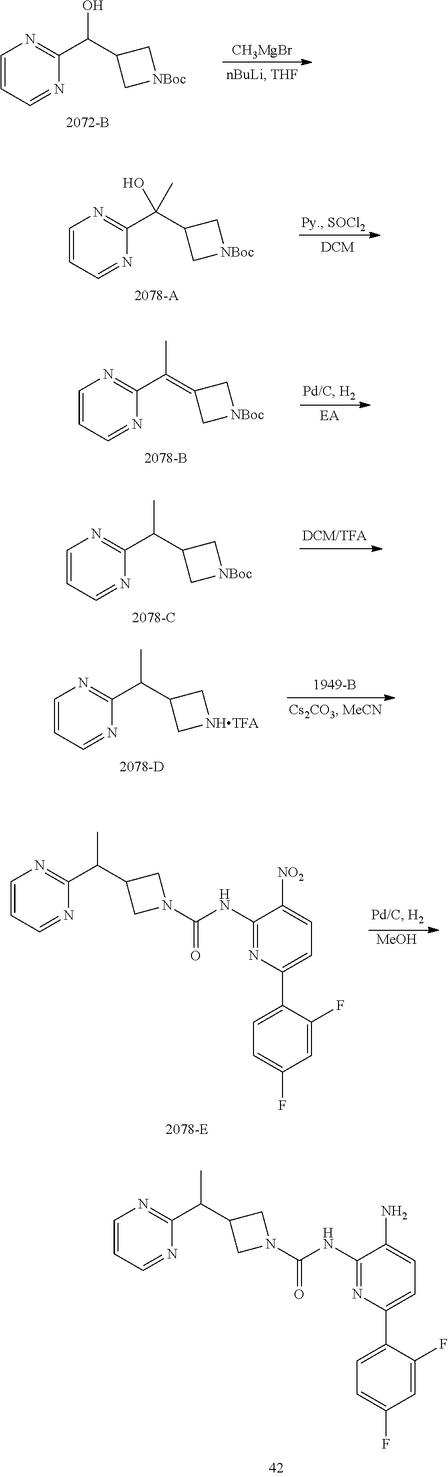

Synthesis of 2078-A. A solution of 2072-B (500 mg, 1.9 mmol) in THF (10 mL) was treated with CH₃MgBr (1.3 mL, 3.80 mmol, solution in THF) dropwise at −78° C. The reaction mixture was then allowed to warm to room temperature, and was stirred at room temperature for 4 h. The mixture was then diluted with saturated aqueous NH₄Cl (20 mL), and extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=10:1˜5:1) to give 2078-A (300 mg, 56%) as a brown oil. MS 280.2 [M+H]⁺.

Synthesis of 2078-B. A solution of 2078-A (300 mg, 1.1 mmol) in DCM (6 mL) was cooled to 0° C. and treated with pyridine (170 mg, 2.15 mmol), followed by dropwise addition of SOCl₂ (128 mg, 1.07 mmol). The reaction mixture was then allowed to warm to room temperature and was stirred at room temperature for 12 h. The mixture was then diluted with water (20 ml), and extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=10:1˜5:1) to give 2078-B (80 mg, 27%) as a brown oil. MS 262.2 [M+H]⁺.

Synthesis of 2078-C. A mixture of 2078-B (80 mg, 0.31 mmol) and Pd/C (40 mg) in EA (6 mL) was stirred at room temperature for 1 h under a H₂ atmosphere. The Pd/C was then removed by filtration through Celite, the filtrate was concentrated, and the residue was purified by Prep-TLC (EtOAc:PE=10:1) to give 2078-C (60 mg, 74%) as a yellow solid. MS 264.2 [M+H]⁺.

Synthesis of 2078-D. To a solution of 2078-C (60 mg, 0.23 mmol) in DCM (3 mL) was added TFA (1 mL) dropwise. The resulting reaction mixture was stirred at room temperature for 1 h, whereupon the solvent was removed in vacuo to give 2078-D as a crude product which was used directly in the next step without further purification.

Synthesis of 2078-E. A mixture of 1949-B (93 mg, 0.19 mmol) and 2078-D (0.23 mmol, crude product from last step) in acetonitrile (10 mL) was treated with Cs₂CO₃ (247 mg, 0.76 mmol), and the reaction mixture was stirred at room temperature for 2 h. The mixture was then diluted with water (20 mL), and extracted with EtOAc (20 mL×3). The combined organic layers were washed with brine (20 mL×3), dried over anhydrous Na₂SO₄ and then concentrated in vacuo. The residue was purified by Prep-TLC (EtOAc:PE=5:1) to give 2078-E (40 mg, 48%) as a yellow solid. MS 441.2 [M+H]⁺.

Synthesis of Compound 42. A mixture of 2078-E (40 mg, 0.09 mmol) and Pd/C (40 mg) in MeOH (5 mL) was stirred at room temperature for 1 h under a H₂ atmosphere. The Pd/C was then removed by filtration through Celite, the filtrate was concentrated, and the residue was purified by Prep-TLC (EtOAc:MeOH=15:1) to give 42 (8.0 mg, 22%) as a yellow solid. MS 411.2 [M+H]⁺.

### Example 9

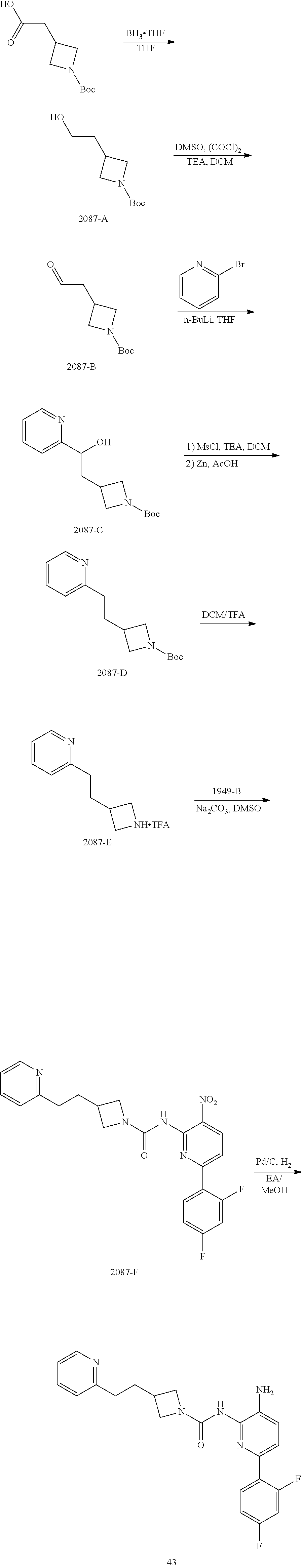

Synthesis of 2087-A. To a solution of 2-(1-(tert-butoxycarbonyl)azetidin-3-yl)acetic acid (5.0 g, 23.3 mmol) in THF (25 mL) was added BH₃.THF (70 mL, 70.0 mmol) dropwise at 0° C. The resulting reaction mixture was stirred at 0° C. for 1 h, whereupon the solution was quenched with water (30 mL) and the solution was stirred at room temperature for 1 h. The THF was removed in vacuo, then the remaining aqueous residue was extracted with EtOAc (20 mL×3), and the combined organic layers were washed with brine (20 mL×3). The organic layer was dried over anhydrous Na₂SO₄ and then concentrated in vacuo. The residue was purified by column chromatography on silica gel (PE:EtOAc=5:1˜1:1) to give 2087-A (4.0 g, 85%) as a colorless oil. MS 146.2 [M-56+H]⁺.

Synthesis of 2087-B. A solution of DMSO (1.17 g, 15.0 mmol) in DCM (10 mL) was treated with (COCl)₂ (1.27 g, 10.0 mmol) at −78° C. dropwise under a N₂ atmosphere. The reaction mixture was stirred at −78° C. for 1 h, whereupon a solution of 2087-A (1.0 g, 5.0 mmol) in DCM (5 mL) was added dropwise, and the reaction mixture continued to stir at −78° C. for 30 min. Finally, TEA (657 mg, 6.5 mmol) was added dropwise to the reaction mixture at −78° C., and the mixture was then allowed to warm to room temperature and stirred for an additional 30 min. The mixture was then diluted with DCM (20 mL) and then washed with water (10 mL×3) and saturated aqueous NaHCO₃ (10 mL×3). The organic layer was dried over anhydrous Na₂SO₄ and then concentrated in vacuo to give 2087-B (900 mg, 83%) as a yellow solid. MS 144.2 [M-56+H]⁺.

Synthesis of 2087-C. A solution of 2-bromopyridine (710 mg, 4.5 mmol) in THF (10 mL) was treated with n-BuLi (2.2 mL, 5.4 mmol) at −78° C. dropwise under a N₂ atmosphere. The resulting reaction mixture was stirred at −78° C. for 1 h, then a solution of 2087-B (900 mg, 4.5 mmol) in THF (5 mL) was added dropwise. The reaction mixture was allowed to warm to room temperature, and was allowed to stir at room temperature for 2 h. The mixture was then quenched with saturated aqueous NH₄Cl (30 mL), and extracted with EtOAc (10 mL×3). The combined organic layers were washed with brine (10 mL×3), then dried over anhydrous Na₂SO₄ and concentrated in vacuo. The residue was purified by column chromatography on silica gel (PE:EtOAc=10:1˜1:1) to give 2087-C (310 mg, 25%) as a yellow solid. MS 279.2 [M+H]⁺.

Synthesis of 2087-D. To a mixture of 2087-C (310 mg, 1.1 mmol) in DCM (10 mL) was added MsCl (192 mg, 1.6 mmol) at 0° C. dropwise, and the reaction mixture as then allowed to warm to room temperature and stirred for 1 h. The mixture was concentrated in vacuo, and the residue was treated with HOAc (8 mL) and zinc dust (429 mg, 6.6 mmol). The resulting mixture was stirred at 40° C. for 3 h, whereupon the solvent was removed in vacuo. The residue was dissolved with EtOAc (30 mL), washed with brine (10 mL×3), and the organic layer was dried over anhydrous Na₂SO₄ and then concentrated in vacuo. The residue was purified by column chromatography on silica gel (PE:EtOAc=10:1˜3:1) to give 2087-D (200 mg, 69%) as a yellow solid. MS 263.2 [M+H]⁺.

Synthesis of 2087-E. To a solution of 2087-D (100 mg, 0.38 mmol) in DCM (3 mL) was added TFA (1 mL) dropwise. The resulting reaction mixture was stirred at room temperature for 1 h, whereupon the solution was concentrated in vacuo to give 2087-E as a crude product which was used directly in the next step without further purification. MS 163.2 [M+H]⁺. Synthesis of 2087-F. A mixture of 2087-E (0.38 mmol, crude product from last step) and 1949-B (104 mg, 0.21 mmol) in DMSO (4 mL) was stirred at room temperature for 10 min, then was treated with Na₂CO₃ (224 mg, 2.11 mmol) and stirred at room temperature for 2 h. The mixture was then diluted with water (10 mL) and extracted with EtOAc (10 mL×3). The combined organic layers were washed with brine (10 mL×3), dried over anhydrous Na₂SO₄ and then concentrated in vacuo. The residue was purified by Prep-TLC (DCM:EtOAc=1:1) to give 2087-F (60 mg, 65%) as a yellow solid. MS 440.2 [M+H]⁺.

Synthesis of Compound 43. A mixture of 2087-F (60 mg, 0.14 mmol) and Pd/C (60 mg) in MeOH/EtOAc (3 mL/3 mL) was stirred at room temperature for 50 min under a H₂ atmosphere. The Pd/C was removed by filtration through Celite, the filtrate was concentrated in vacuo and the residue was purified by Prep-TLC (DCM:MeOH=30:1) to give 43 (18 mg, 31%) as a brown solid. MS 410.2 [M+H]⁺.

### Example 10

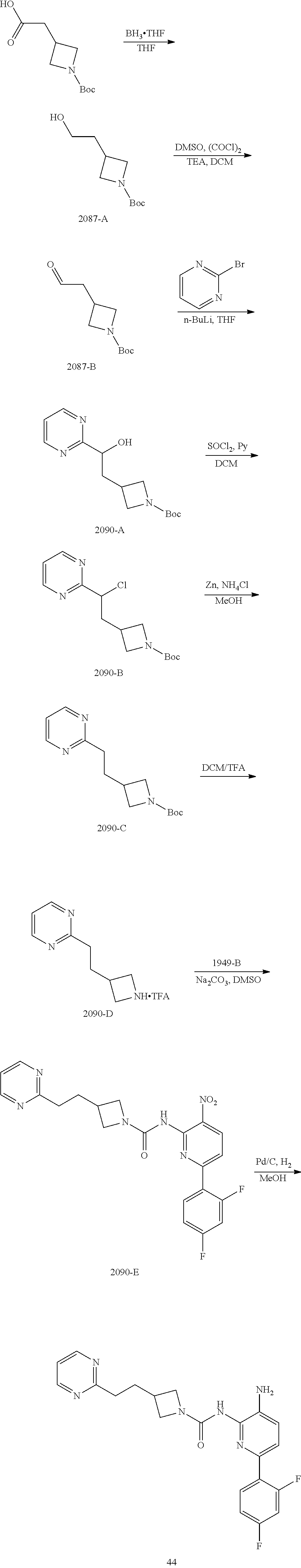

Synthesis of 2087-A. To a solution of 2-(1-(tert-butoxycarbonyl)azetidin-3-yl)acetic acid (5.0 g, 23.3 mmol) in THF (25 mL) was added BH₃.THF (70 mL, 70.0 mmol) dropwise at 0° C. The reaction mixture was stirred at 0° C. for 1 h, whereupon the solution was quenched with water (30 mL), and the mixture was allowed to stir at room temperature for 1 h. The THF was removed in vacuo, then the aqueous residue was extracted with EtOAc (20 mL×3), washed with brine (20 mL×3), and the organic layer was dried over anhydrous Na₂SO₄ and then concentrated in vacuo. The residue was purified by column chromatography on silica gel (PE:EtOAc=5:1˜1:1) to give 2087-A (4.0 g, 85%) as a colorless oil. MS 146.2 [M-56+H]⁺.

Synthesis of 2087-B. A solution of DMSO (1.17 g, 15.0 mmol) in DCM (10 mL) was treated with (COCl)₂ (1.27 g, 10.0 mmol) at −78° C. dropwise under a N₂ atmosphere. The reaction mixture was stirred at −78° C. for 1 h, whereupon a solution of 2087-A (1.0 g, 5.0 mmol) in DCM (5 mL) was added dropwise, and the reaction mixture continued to stir at −78° C. for an additional 30 min. Finally, TEA (657 mg, 6.5 mmol) was added dropwise to the reaction mixture at −78° C., and the mixture was then allowed to warm to room temperature and stirred for an additional 30 min. The mixture was then diluted with DCM (20 mL) and then washed with water (10 mL×3) and saturated aqueous NaHCO₃ (10 mL×3). The organic layer was dried over anhydrous Na₂SO₄ and then concentrated in vacuo to give 2087-B (900 mg, 83%) as a yellow solid. MS 144.2 [M-56+H]⁺.

Synthesis of 2090-A. A solution of 2-bromopyrimidine (715 mg, 4.5 mmol) in THF (10 mL) was treated with n-BuLi (2.2 mL, 5.4 mmol) at −78° C. dropwise under a N₂ atmosphere. The resulting reaction mixture was stirred at −78° C. for 1 h, whereupon a solution of 2087-B (900 mg, 4.5 mmol) in THF (5 mL) was added dropwise. The reaction mixture was then allowed to warm to room temperature, and was stirred at room temperature for 2 h. The mixture was then quenched with saturated aqueous NH₄Cl (30 mL) extracted with EtOAc (10 mL×3), and the combined organic layers were washed with brine (10 mL×3). The organic layer was then dried over anhydrous Na₂SO₄ and concentrated in vacuo. The residue was purified by column chromatography on silica gel (PE:EtOAc=10:1˜1:1) to give 2090-A (320 mg, 25%) as a yellow solid. MS 280.2 [M+H]⁺.

Synthesis of 2090-B. A mixture of 2090-A (320 mg, 1.1 mmol) and pyridine (521 mg, 6.6 mmol) in DCM (10 mL) was cooled to 0° C. and treated with SOCl₂ (196 mg, 1.7 mmol) dropwise, and the reaction mixture was then warmed to room temperature and stirred for 4 h. The reaction mixture was then diluted with EtOAc (30 mL), and washed with brine (10 mL×3). The organic layer was dried over anhydrous Na₂SO₄ and then concentrated in vacuo. The residue was purified by column chromatography on silica gel (PE:EtOAc=10:1˜3:1) to give 2090-B (160 mg, 69%) as a yellow solid. MS 298.2 [M+H]⁺.

Synthesis of 2090-C. A solution of 2090-B (160 mg, 0.54 mmol) in MeOH (6 mL) was treated with zinc dust (60 mg, 1.1 mmol) and NH₄Cl (58 mg, 1.1 mmol). The resulting reaction mixture was stirred at room temperature for 16 h, whereupon the reaction was diluted with EtOAc (30 mL), and washed with brine (10 mL×3). The organic layer was dried over anhydrous Na₂SO₄ and then concentrated in vacuo. The residue was purified by column chromatography on silica gel (PE:EtOAc=10:1˜2:1) to give 2090-C (70 mg, 49%) as a yellow solid. MS 264.2 [M+H]⁺.

Synthesis of 2090-D. To a solution of 2090-C (70 mg, 0.27 mmol) in DCM (3 mL) was added TFA (1 mL) dropwise, and the reaction mixture was stirred at room temperature for 1 h. The reaction mixture was then concentrated in vacuo to give 2090-D as a crude product which was used directly in the next step without further purification. MS 164.2 [M+H]⁺. Synthesis of 2090-E. A mixture of 2090-D (0.27 mmol, crude product from last step) and 1949-B (74 mg, 0.15 mmol) in DMSO (6 mL) was stirred at room temperature for 10 min, then was treated with Na₂CO₃ (159 mg, 1.50 mmol), and the reaction mixture was stirred at room temperature for 2 h. The mixture was then diluted with water (10 mL) and extracted with EtOAc (10 mL×3). The combined organic layers were washed with brine (10 mL×3), dried over anhydrous Na₂SO₄ and then concentrated in vacuo. The residue was purified by Prep-TLC (PE:EtOAc=1:4) to give 2090-E (40 mg, 61%) as a yellow solid. MS 441.2 [M+H]⁺.

Synthesis of Compound 44. A mixture of 2090-E (40 mg, 0.09 mmol) and Pd/C (40 mg) in MeOH (4 mL) was stirred at room temperature for 30 min under a H₂ atmosphere. The Pd/C was removed by filtration through Celite, the filtrate was concentrated in vacuo and the residue was purified by Prep-TLC (DCM:MeOH=30:1) to give 44 (5 mg, 14%) as a brown solid. MS 411.2 [M+H]⁺.

### Example 11

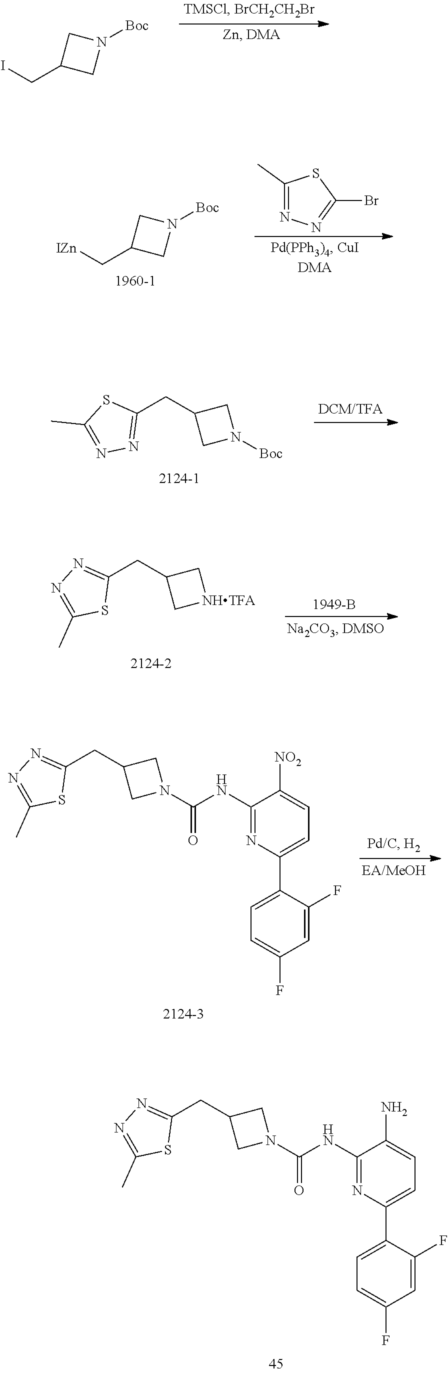

Synthesis of 1960-1. To a mixture of zinc dust (230 mg, 3.54 mmol) in anhydrous DMA (0.8 mL) was added TMSCl and 1,2-dibromoethane (0.06 mL, v/v=7/5), and the reaction mixture was stirred at room temperature for 20 min under a N₂ atmosphere. A solution of tert-butyl 3-(iodomethyl)azetidine-1-carboxylate (800 mg, 2.70 mmol) in anhydrous DMA (1 mL) was then added, and the resulting mixture was stirred at room temperature for 16 h under a N₂ atmosphere. The reaction mixture was used directly in the next step as 1960-1. The concentration of 1960-1 was about 1.0 mol/L in DMA.

Synthesis of 2124H. A mixture of 2-bromo-5-methyl-1,3,4-thiadiazole (297 mg, 1.67 mmol), CuI (32 mg, 0.17 mmol) and Pd(PPh₃)₄ (96 mg, 0.084 mmol) in anhydrous DMA (6 mL) under a N₂ atmosphere was treated with 1960-1 (2.0 mL). The resulting reaction mixture was stirred at 60° C. for 48 h under a N₂ atmosphere. The mixture was then diluted with water (30 mL) and extracted with EtOAc (20 mL×3). The combined organic layers were washed with brine (20 mL×3), dried over anhydrous Na₂SO₄ and then concentrated in vacuo. The residue was purified by Prep-TLC (EtOAc:PE=1:1) to give 2124-1 (120 mg, 27%) as a yellow solid. MS 270.3 [M+H]⁺, 214.2 [M-55]⁺

Synthesis of 2124-2. To a solution of 2124-1 (120 mg, 0.45 mmol) in DCM (10 mL) was added TFA (3 mL) dropwise. The reaction mixture was stirred at room temperature for 1 h, whereupon it was concentrated in vacuo to give 2124-2 as a crude product which was used directly in the next step without further purification. MS 170.3 [M+H]⁺.

Synthesis of 2124-3. A mixture of 2124-2 (0.45 mmol, crude product from last step) and Na₂CO₃ (477 mg, 4.5 mmol) in DMSO (10 mL) was stirred at room temperature for 10 min, then 1949-B (123 mg, 0.25 mmol) was added, and the reaction mixture was stirred at room temperature for 2 h. The mixture was then diluted with water (30 mL) and extracted with EtOAc (20 mL×3). The combined organic layers were washed with brine (20 mL×3), dried over anhydrous Na₂SO₄ and then concentrated in vacuo. The residue was purified by Prep-TLC (EtOAc) to give 2124-3 (30 mg, 15%) as a yellow solid. MS 447.0 [M+H]⁺.

Synthesis of Compound 45. A mixture of 2124-3 (30 mg, 0.067 mmol) and Pd/C (30 mg) in MeOH/EtOAc (5 mL/5 mL) was stirred at room temperature for 1 h under a H₂ atmosphere. The Pd/C was removed by filtration through Celite, the filtrate was concentrated in vacuo and the residue was purified by Prep-TLC (EtOAc:MeOH=14:1) to give 45 (14 mg, 54%) as an off-white solid. MS 417.0 [M+H]⁺.

Compound 46 was synthesized in a similar manner using an appropriately substituted aryl bromide variant of reagents used to synthesize 45.

Compound 46.1 mg, 53%, an off-white solid.

### Example 12

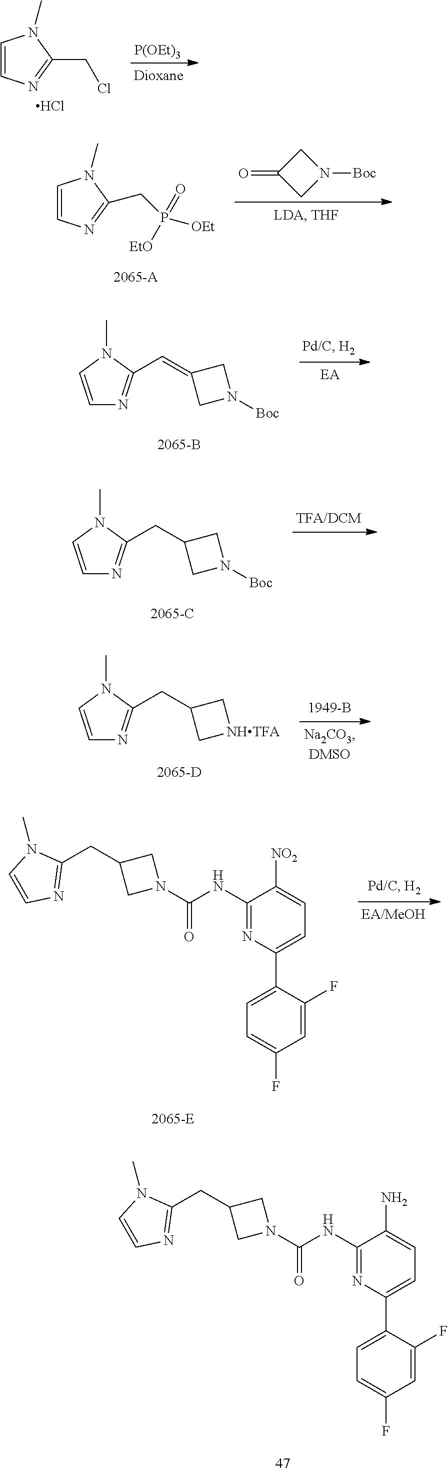

Synthesis of 2065-A, A solution of 2-(chloromethyl)-1-methyl-1H-imidazole hydrochloride (2.0 g, 12.0 mmol) and P(OEt)₃ (20 mL) in dioxane (20 mL) was stirred at 120° C. for 4 h under N₂. The mixture was then concentrated in vacuo, and the residue was purified by column chromatography on silica gel (EtOAc:PE=1:1 to EtOAc:MeOH=6:1) to give 2065-A (760 mg, 27%) as a colorless oil. MS 233.2 [M+H]⁺.

Synthesis of 2065-B. A solution of 2065-A (200 mg, 0.86 mmol) in THF (5 mL) was cooled to −78° C. and then LDA (2.6 mL, 2.6 mmol) was added dropwise under a N₂ atmosphere. The solution was stirred at −78° C. for 1 h, whereupon a solution of tert-butyl 3-oxoazetidine-1-carboxylate (192 mg, 1.1 mmol) in THF (3 mL) was added dropwise. The reaction was then allowed to warm to room temperature, and was stirred at room temperature for 16 h. The mixture was then quenched with saturated aqueous NH₄Cl (30 mL) and extracted with EtOAc (10 mL×3). The combined organic layers were washed with brine (10 mL×3), dried over anhydrous Na₂SO₄ and then concentrated in vacuo. The residue was purified by Prep-TLC (PE:EtOAc=1:3) to give 2065-B (80 mg, 37%) as a yellow oil. MS 250.2 [M+H]⁺.

Synthesis of 2065-C. A mixture of 2065-B (200 mg, 0.80 mmol) and Pd/C (200 mg) in EtOAc (10 mL) was stirred at room temperature for 1 h under a H₂ atmosphere. The Pd/C was then removed by filtration through Celite, the filtrate was concentrated and the residue was purified by Prep-TLC (EtOAc) to give 2065-C (120 mg, 60%) as a yellow solid. MS 152.3 [M-100+H]⁺.

Synthesis of 2065-D. To a solution of 2065-C (120 mg, 0.48 mmol) in DCM (3 mL) was added TFA (1 mL) dropwise. The reaction mixture was stirred at room temperature for 1 h, whereupon the solvent was removed in vacuo to give 2065-D as a crude product which was used directly in the next step without further purification. MS 152.3 [M+H]⁺.

Synthesis of 2065-E. A mixture of 1949-B (132 mg, 0.27 mmol) and 2065-D (0.48 mmol, crude product from last step) in DMSO (5 mL) was stirred at room temperature for 10 min, then was treated with Na₂CO₃ (286 mg, 2.7 mmol), and the reaction mixture was stirred at room temperature for 2 h. The mixture was then diluted with water (10 mL), and extracted with EtOAc (10 mL×3). The combined organic layers were washed with brine (10 mL×3), dried over anhydrous Na₂SO₄ and then concentrated in vacuo. The residue was purified by Prep-TLC (EtOAc:MeOH=50:1) to give 2065-E (80 mg, 69%) as a yellow solid. MS 429.0 [M+H]⁺.

Synthesis of Compound 47. A mixture of 2065-E (80 mg, 0.19 mmol) and Pd/C (80 mg) in EtOAc/MeOH (3 mL/3 mL) was stirred at room temperature for 1 h under a H₂ atmosphere. The Pd/C was then removed by filtration through Celite, the filtrate was concentrated and the residue was purified by Prep-TLC (EA:MeOH=15:1) to give 47 (40 mg, 53%) as a white solid. MS 199.1 [M/2+H]⁺, MS 399.0 [M+H]⁺.

### Example 13 Synthesis of Compound 51 and 52

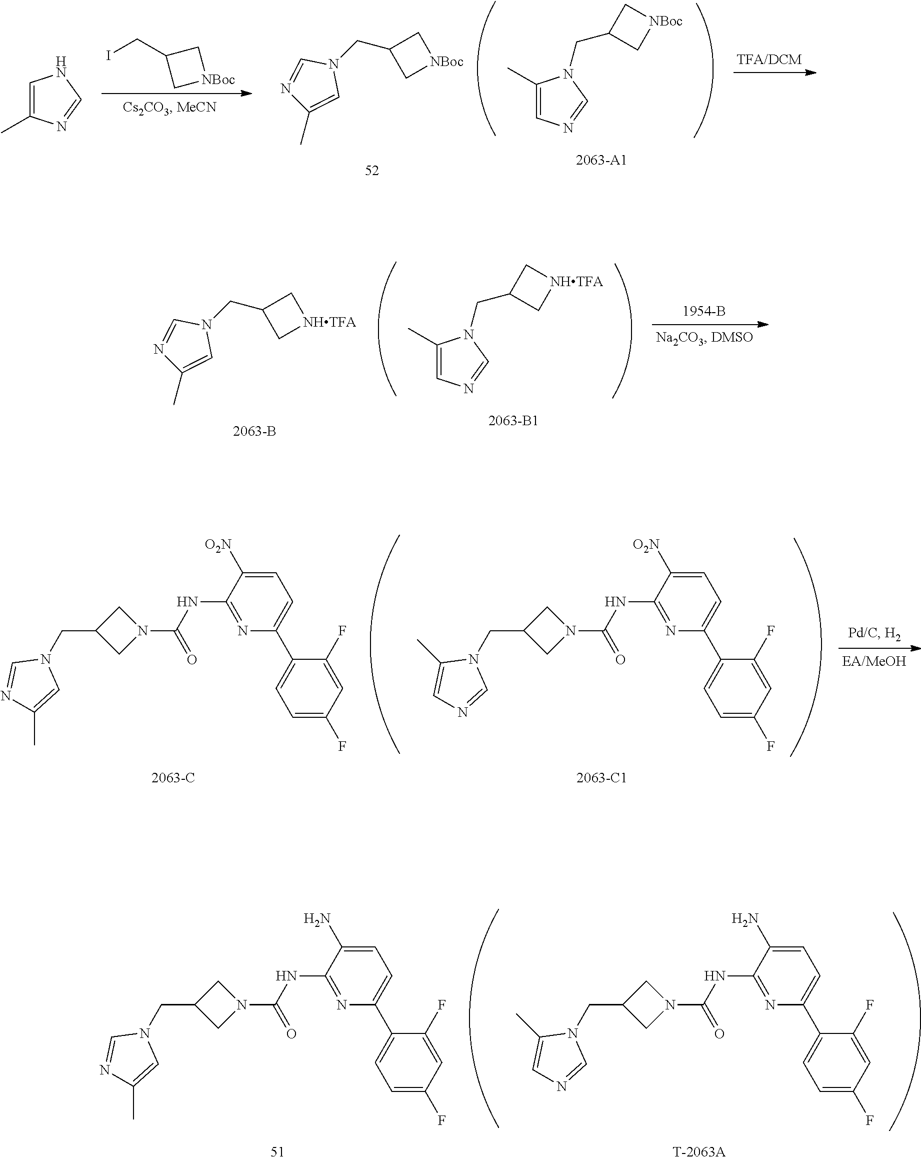

Synthesis of 2063-A and 2063-A1. A mixture of tert-butyl 3-(iodomethyl)-azetidine-1-carboxylate (419 mg, 1.41 mmol), 4-methyl-1H-imidazole (97 mg, 1.18 mmol) and Cs₂CO₃ (769 mg, 2.36 mol) in acetonitrile (10 mL) was stirred at 80° C. for 3 h. The mixture was diluted with water (30 mL) and extracted with EtOAc (10 mL×3). The combined organic layers were washed with brine (10 mL×3), dried over anhydrous Na₂SO₄ and then concentrated in vacuo. The residue was purified by column chromatography on silica gel (PE:EtOAc=10:1 to 1:1) to give a mixture of 2063-A and 2063-A1 (231 mg, 78%) as a yellow oil. MS 239.7 [M+H]⁺.

Synthesis of 2063-B and 2063-B1. To a solution of 2063-A and 2063-A1 (201 mg, 0.80 mmol) in DCM (6 mL) was added TFA (2 mL) dropwise at 0° C. Then the solution was stirred at room temperature for 1 h. The solution was concentrated in vacuo to give a mixture of 2063-B and 2063-B1 as a crude product which was directly used in the next step. MS 151.1 [M+H]⁺.

Synthesis of 2063-C and 2061-C1. A mixture of 2063-B and 2063-B1 (0.80 mmol, crude product from previous step), 1949-B (216 mg, 0.44 mmol) in DMSO (6 mL) was stirred at room temperature for 10 min, then Na₂CO₃ (471 mg, 4.44 mmol) was added into above mixture and stirred at room temperature for 2 h. The mixture was diluted with water (20 mL) and extracted with EtOAc (10 mL×3). The combined organic layers were washed with brine (10 mL×3), dried over anhydrous Na₂SO₄ and then concentrated in vacuo. The residue was purified by Prep-TLC (DCM:MeOH=45:1) to give a mixture of 2063-C and 2063-C1 (109 mg, 58%) as a yellow solid. MS 429.1 [M+H]⁺.

Synthesis of 51 and 52. A mixture of 2063-C and 2063-C1 (124 mg, 0.29 mmol), Pd/C (124 mg) in MeOH/EtOAc (5 mL/5 mL) was stirred at room temperature for 2 h under H₂ atmosphere. Pd/C was removed by filtration through a pad of Celite. The filtrate was concentrated in vacuo and the residue was purified by Prep-TLC (DCM:MeOH=25:1) to give a mixture of 51 and 52 (90 mg, 78%) as a white solid. MS 399.1 [M+H]⁺. Separation of 51 and 52. The mixture of 51 and 52 (90 mg, 0.23 mmol) was separated by using SFC (Column: Chiralcel OJ-3; Solvent: EtOH (0.3% DEA); Flow rate: 2 mL/min; RT₅₁=1.138 min, RT₅₂=1.920 min) to give 51 (40 mg, 44%) as a white solid (MS 399.1 [M+H]⁺) and 52 (25 mg, 28%) as a white solid. MS 399.1 [M+H]⁺.

Compounds 53 and 54 were synthesized in a similar manner as 51 and 52 by using 3-methyl-1H-pyrazole as a reagent.

Compound 53. 45 mg, 46%, a yellow solid.

Compound 54. 24 mg, 25%, a yellow solid.

### Example 14 Synthesis of Compound 55

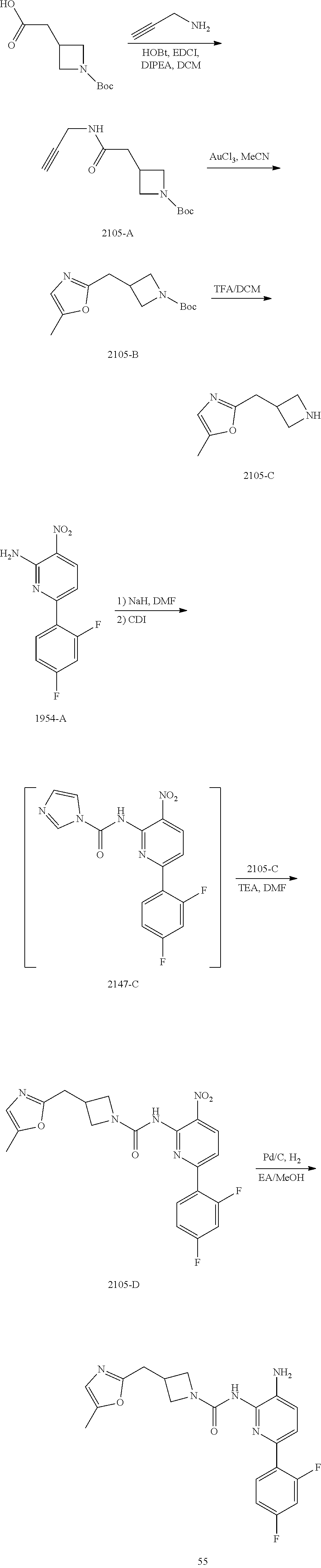

Synthesis of 2105-A. To a solution of 2-(1-(tert-butoxycarbonyl)azetidin-3-yl)acetic acid (3.23 g, 15 mmol), HOBt (2.43 g, 18 mmol) and EDCI (4.32 g, 22.5 mmol) in DCM (40 mL) was added DIPEA (2.58 g, 30 mmol) and stirred at room temperature for 30 min under nitrogen atmosphere. Then a solution of prop-2-yn-1-amine (1.650 g, 30 mmol) in DCM (10 mL) was added into above mixture and stirred at room temperature for 24 h. The mixture was diluted with DCM (200 mL), washed with 0.5 N HCl (100 mL×2), saturated NaHCO₃ (100 mL×2) and brine (100 mL×2). The organic layer was dried over anhydrous Na₂SO₄ and then concentrated in vacuo. The residue was purified by column chromatography on silica gel (PE:EtOAc=10:1 to 2:1) to give 2105-A (3.1 g, 82%) as color oil. MS 197.0 [M-55]⁺.

Synthesis of 2105-B. To a solution of 2105-A (2.0 g, 7.9 mmol) in acetonitrile (20 mL) was added gold trichloride (200 mg, 0.66 mmol) and stirred at 45° C. for 84 h under nitrogen atmosphere. The mixture was concentrated in vacuo. The residue was purified by column chromatography on silica gel (PE:EtOAc=10:1 to 1:4) to give 2105-B (1.1 g, 55%) as colorless oil. MS 197.0 [M-55]⁺.

Synthesis of 2105-C. To a solution of 2105-B (300 mg, 1.2 mmol) in DCM (12 mL) was added TFA (4 mL) drop wise at 0° C. Then the solution was stirred at room temperature for 1 h. The solution was concentrated in vacuo to give 2105-C as a crude product. Then the residue was dissolved in DMF (6 mL) and treated with TEA (363 mg, 3.6 mmol) to give 2105-C as a solution which was directly used in the next step. MS 153.0 [M+H^{]+}.

Synthesis of 2105-D. To a solution of 1949-A (200 mg, 0.8 mmol) in DMF (5 mL) was added NaH (60% in mineral oil) (80 mg, 2.0 mmol) at ice bath and the mixture was stirred at ice bath for 30 min, then CDI (162 mg, 1.0 mmol) was added into above mixture and stirred at ice bath for another 30 min. Finally, the solution of 2105-C was added into above mixture at ice bath and stirred at ice bath for 1 h. The mixture was quenched with water (50 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=10:1 to 1:4) to give 2105-D (220 mg, 51%) as a yellow solid. MS 430.0 [M+H]⁺.

Synthesis of 55. A mixture of 2105-D (200 mg, 0.47 mmol) and Pd/C (200 mg) in MeOH/EtOAc (10 mL/10 mL) were stirred at room temperature for 120 min under H₂ atmosphere. Pd/C was removed by filtration through a pad of Celite. The filtrate was concentrated in vacuo and the residue was purified by Prep-HPLC to give 55 (95 mg, 51%) as a white solid. MS 400.0 [M+H]⁺.

Compounds 62 and 63 were synthesized in a similar manner as 55 by using 2332-E or 2475-E, respectively, as reagents in place of 2147-C.

Compound 62. 70 mg, 15%, a yellow solid.

Compound 63. 90 mg, 44%, a white solid.

### Example 15 Synthesis of Compound 56

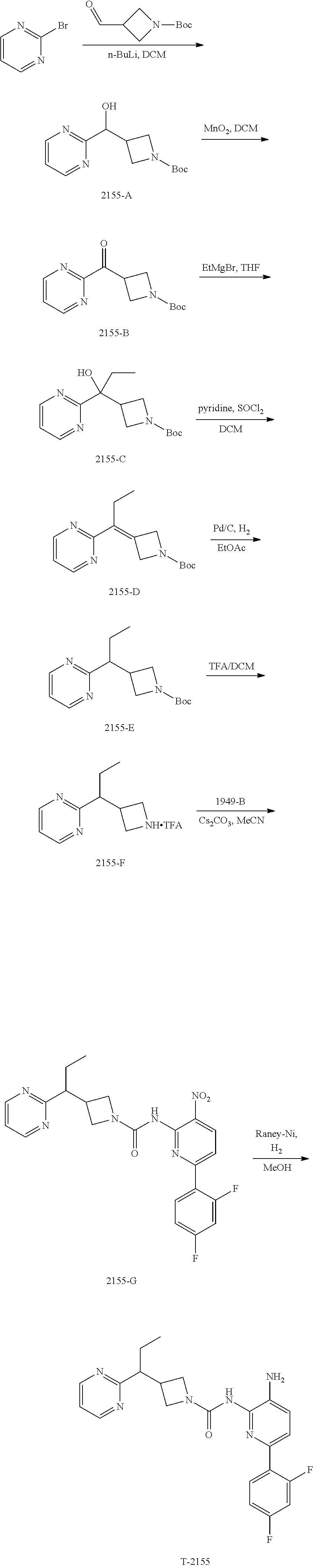

Synthesis of 2155-A. To a solution of 2-bromopyrimidine (1.0 g, 6.29 mmol) in DCM (20 mL) was added n-BuLi (3.0 mL, 7.55 mmol) dropwise at −78° C. and stirred at −78° C. for 1 h under nitrogen atmosphere. Then a solution of tert-butyl 3-formylazetidine-1-carboxylate (1.4 g, 7.55 mmol) in DCM (10 mL) was added into the above mixture dropwise at −78° C. The resulting mixture was warmed to room temperature for 3 h. The mixture was quenched with saturated NH₄Cl (40 mL), extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=10:1 to EtOAc) to give 2155-A (300 mg, 18%) as a light yellow solid. MS 266.2 [M+H]⁺.

Synthesis of 2155-B. To a solution of 2155-A (300 mg, 1.13 mmol) in DCM (20 mL) was added MnO₂ (3.0 g). Then the solution was stirred at room temperature for 4 h. MnO₂ was removed by filtration through a pad of Celite. The filtrate was concentrated and the residue was purified by Prep-TLC (EtOAc:PE=10:1) to give 2155-B (150 mg, 50%) as a light yellow solid. MS 264.2 [M+H]⁺.

Synthesis of 2155-C. To a solution of 2155-B (3.4 g, 12.9 mmol) in THF (40 mL) was added ethylmagnesium bromide (8.6 mL, 25.8 mmol) dropwise at −78° C. and then warmed to room temperature for 4 h under nitrogen atmosphere. The mixture was quenched with saturated NH₄Cl (50 mL), extracted with EtOAc (50 mL×3). The combined organic layers were washed with brine (50 mL×3), dried over anhydrous Na₂SO₄ and then concentrated in vacuo. The residue was purified by column chromatography on silica gel (PE:EtOAc=10:1 to 5:1) to give 2155-C (340 mg, 9%) as a brown oil. MS 294.2 [M+H]⁺.

Synthesis of 2155-D. A solution of 2155-C (340 mg, 1.2 mmol) in DCM (10 mL) was treated with pyridine, cooled to 0° C. (187 mg, 2.4 mmol), and then SOCl₂ (143 mg, 1.2 mmol) was added dropwise. The reaction was then warmed to room temperature and stirred for 12 h. The mixture was diluted with water (20 ml), and extracted with EtOAc (40 mL×3). The combined organic layers were washed with brine (40 mL×3), dried over anhydrous Na₂SO₄ and then concentrated in vacuo. The residue was purified by column chromatography on silica gel (PE:EtOAc=10:1 to 5:1) to give 2155-D (200 mg, 60%) as a brown oil. MS 276.2 [M+H]⁺.

Synthesis of 2155-E A mixture of 2155-D (200 mg, 0.73 mmol) and Pd/C (200 mg) in EtOAc (10 mL) was stirred at room temperature for 1 h under a H₂ atmosphere. Pd/C was then removed by filtration through a pad of Celite. The filtrate was concentrated and the residue was purified by Prep-TLC (EA:PE=10:1) to give 2155-E (100 mg, 49%) as yellow solid. MS 278.2 [M+H]⁺.

Synthesis of 2155-F. A solution of 2155-E (200 mg, 0.36 mmol) in DCM (10 mL) was cooled to 0° C. and TFA (4 mL) was added dropwise. The reaction was allowed to warm to room temperature and was stirred at room temperature for 1 h. The solvent was removed in vacuo to give 2155-F as a crude product which was used directly in the next step.

Synthesis of 2155-G. To a mixture of 1949-B (147 mg, 0.3 mmol) and 2078-D (0.36 mmol, crude product from previous step) in acetonitrile (10 mL) was added Cs₂CO₃ (391 mg, 1.2 mmol) and then stirred at room temperature for 2 h. The mixture was diluted with water (20 mL), extracted with EtOAc (20 mL×3). The combined organic layers were washed with brine (20 mL×3), dried over anhydrous Na₂SO₄ and then concentrated in vacuo. The residue was purified by Prep-TLC (EA:PE=5:1) to give 2155-G (70 mg, 51%) as a yellow solid. MS 455.2 [M+H]⁺.

Synthesis of 56. A mixture of 2155-G (70 mg, 0.15 mmol) and Raney-Ni (70 mg) in MeOH (6 mL) was stirred at room temperature for 1 h under H₂ atmosphere. Raney-Ni was then removed by filtration through a pad of Celite. The filtrate was concentrated and the residue was purified by Prep-TLC (EA:MeOH=15:1) to give 56 (35 mg, 55%) as yellow solid. MS 425.2 [M+H]⁺.

Compound 57 was synthesized in a similar manner as 56 by using methyl magnesium bromide and 2475-E. Compound 58 was synthesized in a similar manner to 56 by using an appropriately substituted boronic acid when making the 2-F-phenyl core in place of 1949-B.

Compound 57. A mg, 17%, an orange solid.

Compound 58. 75 mg, 67%, a flesh color solid.

### Example 16 Synthesis of Compound 59

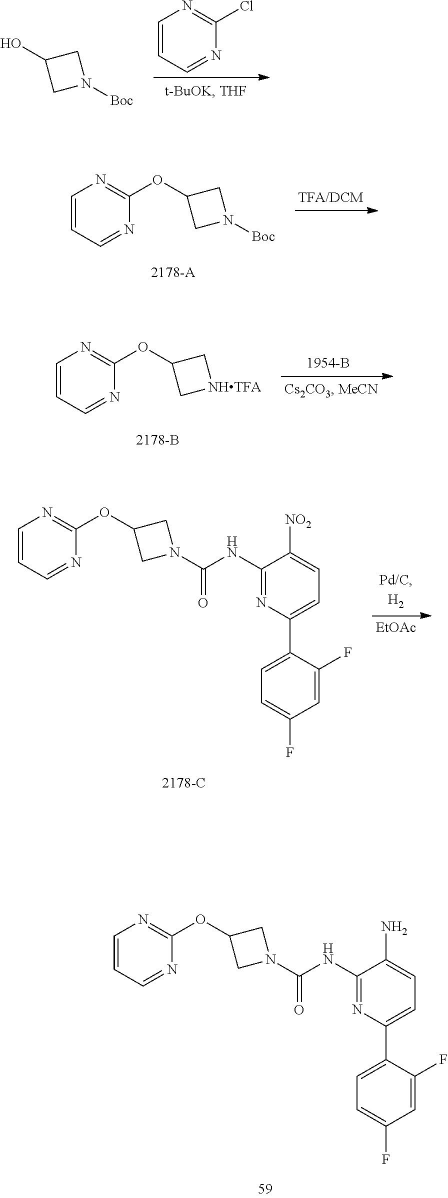

Synthesis of 2178-A. To a mixture of tert-butyl 3-hydroxyazetidine-1-carboxylate (300 mg, 1.73 mmol) and 2-chloropyrimidine (413 mg, 2.38 mmol) in THF (10 mL) was added t-BuOK (401 mg, 3.57 mmol). The mixture was stirred at 65° C. for 6 h and then concentrated in vacuo. The residue was dissolved with EtOAc (20 mL) and the solution was washed with brine (10 mL×3). The organic layer was dried over anhydrous Na₂SO₄ and then concentrated in vacuo. The residue was purified by column chromatography on silica gel (PE:EtOAc=10:1 to 5:1) to give 2178-A (350 mg, 53%) as a yellow oil. MS 252.2 [M+H]⁺.

Synthesis of 2178-B. A of 2178-A (350 mg, 1.40 mmol) in DCM (9 mL) was cooled to 0° C. and TFA (3 mL) was added dropwise. The reaction was allowed to warm to room temperature and was stirred at room temperature for 1 h. The solution was then concentrated in vacuo to give 2178-B as a crude product which was used directly in the next step. MS 196.0 [M+H]⁺.

Synthesis of 2178-C. A mixture of 2178-B (1.40 mmol, crude product from previous step) and 1949-B (326 mg, 0.66 mmol) in acetonitrile (6 mL) was stirred at room temperature for 10 min, then Cs₂CO₃ (649 mg, 1.99 mmol) was added and the reaction mixture was stirred at room temperature for 2 h. The mixture was then diluted with water (30 mL) and extracted with EtOAc (10 mL×3). The combined organic layers were washed with brine (10 mL×3), dried over anhydrous Na₂SO₄ and then concentrated in vacuo. The residue was purified by Prep-TLC (PE:EtOAc=3:2) to give 2178-C (100 mg, 35%) as a yellow oil. MS 429.0 [M+H]⁺.

Synthesis of 59. A mixture of 2178-C (100 mg, 0.23 mmol) and Pd/C (100 mg) in MeOH/EtOAc (50 mL/50 mL) was stirred at room temperature for 1 h under a H₂ atmosphere. Pd/C was removed by filtration through a pad of Celite. The filtrate was concentrated in vacuo and the residue was purified by Prep-TLC (EtOAc) to give 59 (75 mg, 73%) as a pale yellow solid. MS 399.0 [M+H]⁺.

### Example 17 Synthesis of 60

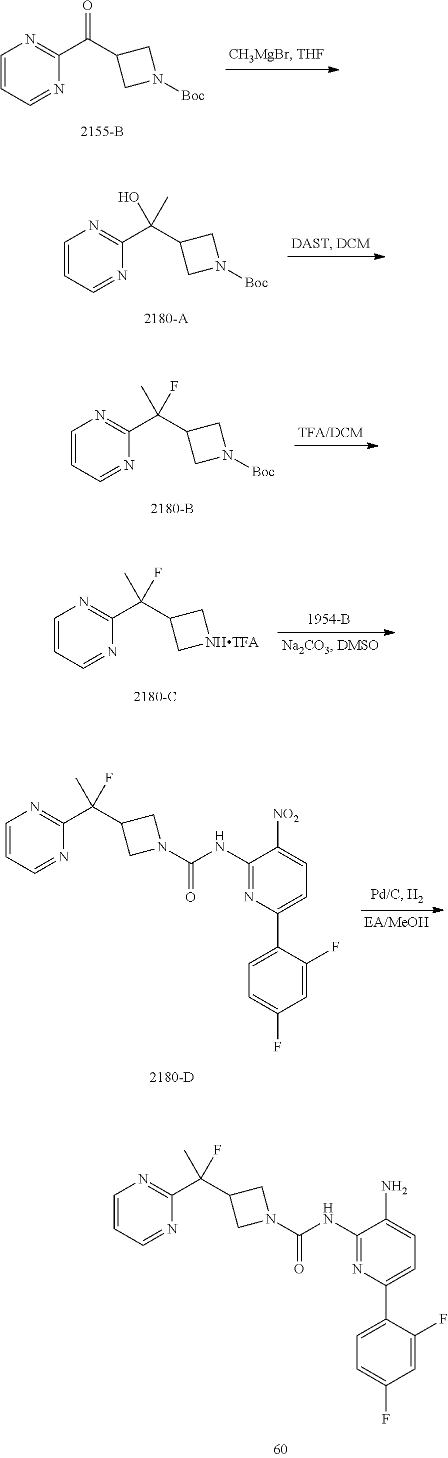

Synthesis of 2180-A. To a solution of 2155-B (1.1 g, 4.2 mmol) in THF (40 mL) was added methylmagnesium bromide (2.8 mL, 8.4 mmol) dropwise at −78° C. and then warmed to room temperature for 4 h under nitrogen atmosphere. The mixture was quenched with saturated NH₄Cl (50 mL), extracted with EtOAc (50 mL×3). The combined organic layers were washed with brine (50 mL×3), dried over anhydrous Na₂SO₄ and then concentrated in vacuo. The residue was purified by column chromatography on silica gel (PE:EtOAc=10:1 to 5:1) to give 2180-A (500 mg, 43%) as a brown oil. MS 280.2 [M+H]⁺.

Synthesis of 2180-B. To a solution of 2180-A (200 mg, 0.72 mmol) in DCM (10 mL) was added DAST (0.4 ml) dropwise at −78° C. under nitrogen atmosphere and then warmed to room temperature for 1 h. The mixture was quenched with saturated NaHCO₃ (50 mL), extracted with DCM (50 mL×3). The combined organic layers were washed with brine (50 mL×3), dried over anhydrous Na₂SO₄ and then concentrated in vacuo. The residue was purified by Prep-TLC (PE:EtOAc=1:3) to give 2180-B (80 mg, 40%) as a brown solid. MS 282.2 [M+H]⁺.

Synthesis of 2180-C. To a solution of 2180-B (80 mg, 0.28 mmol) in DCM (3 mL) was added TFA (1 mL) dropwise at 0° C. The reaction mixture was allowed to warm to room temperature and was stirred at room temperature for 1 h. The solvent was then removed in vacuo to give 2180-C as a crude product which was used directly in the next step. MS 182.2 [M+H]⁺.

Synthesis of 2180-D. To a mixture of 1954-B (92 mg, 0.18 mmol) and 2180-C (0.28 mmol) in DMSO (20 mL) was added Na₂CO₃ (190 mg, 1.8 mmol). The resulting mixture was stirred at room temperature for 2 h. The mixture was then diluted with water (50 mL), and extracted with EtOAc (50 mL×3). The combined organic layers were washed with brine (50 mL×3), dried over anhydrous Na₂SO₄ and then concentrated in vacuo. The residue was purified by Prep-TLC (PE:EtOAc=5:1) to give 2180-D (40 mg, 48%) as a yellow solid. MS 459.2 [M+H]⁺. Synthesis of 60. A mixture of 2180-D (40 mg, 0.09 mmol) and Pd/C (40 mg) in MeOH/EtOAc (3 mL/3 mL) was stirred at room temperature for 1 h under a H₂ atmosphere. Pd/C was removed by filtration through a pad of Celite. The filtrate was concentrated and the residue was purified by Prep-TLC (EtOAc:MeOH=15:1) to give 60 (10 mg, 26%) as a yellow solid. MS 429.2 [M+H]⁺.

### Example 18 Synthesis of Compound 61

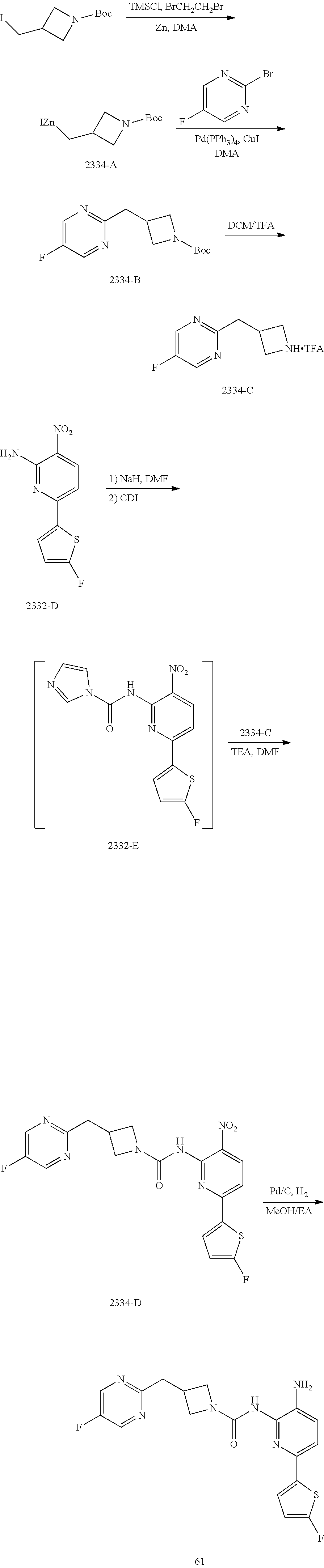

Synthesis of 2334-A. To a mixture of zinc dust (3.87 g, 59.5 mmol) in anhydrous DMA (16 mL) was added TMSCl and 1,2-dibromoethane (0.96 mL, v/v=7/5) and the reaction mixture stirred at room temperature for 20 min under a nitrogen atmosphere. A solution of tert-butyl 3-(iodomethyl)azetidine-1-carboxylate (13.6 g, 45.8 mmol) in anhydrous DMA (16 mL) was then added, and the resulting mixture was stirred at room temperature for 16 h under a nitrogen atmosphere. The mixture was used directly in the next step as 2334-A. The concentration of 2334-A was about 1.0 mol/L in DMA.

Synthesis of 2334-B. A mixture of 2-bromo-5-fluoropyrimidine (6.0 g, 33.9 mmol), CuI (646 mg, 3.4 mmol) and Pd(PPh₃)₄ (1.96 g, 1.7 mmol) in anhydrous DMA (100 mL) under a nitrogen atmosphere was treated with 2334-A (34.0 mL). The resulting mixture was stirred at 60° C. for 48 h under a nitrogen atmosphere. The mixture was then diluted with water (400 mL) and extracted with EtOAc (200 mL×3). The combined organic layers were washed with brine (200 mL×3), dried over anhydrous Na₂SO₄ and then concentrated in vacuo. The residue was purified by column chromatography on silica gel (PE:EtOAc=20:1 to 10:1) to give 2334-B (6.3 g, 70%) as a yellow solid. MS 212.1 [M-55]⁺.

Synthesis of 2334-C. To a solution of 2334-B (720 mg, 2.70 mmol) in DCM (21 mL) was added TFA (7 mL) drop wise at 0° C. Then the solution was stirred at room temperature for 1 h. The solution was concentrated in vacuo and the residue was dissolved in DMF (6 mL) and treated with TEA (818 mg, 8.1 mmol) to give 2334-C as a solution which was directly used in the next step. MS 168.1 [M+H]⁺.

Synthesis of 2334-D. A solution of 2332-D (540 mg, 2.26 mmol) in DMF (6 mL) was cooled to 0° C. and treated with NaH (60% in mineral oil) (181 mg, 4.52 mmol). The reaction mixture was stirred at 0° C. for 30 min, then CDI (305 mg, 1.88 mmol) was added and stirring continued at 0° C. for another 30 min. Finally, the solution of 2334-C was added into above mixture at ice bath and stirred at ice bath for 1 h. The mixture was quenched with water (50 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (DCM:EtOAc=10:1 to 2:1) to give 2334-D (390 mg, 40%) as a yellow solid. MS 433.1 [M+H]⁺.

Synthesis of 61. A mixture of 2334-D (390 mg, 0.90 mmol) and Pd/C (390 mg) in MeOH/EtOAc (10 mL/10 mL) was stirred at room temperature for 50 min under H₂ atmosphere. Pd/C was removed by filtration through a pad of Celite. The filtrate was concentrated in vacuo and the residue was purified by Prep-HPLC to give 61 (230 mg, 63%) as a white solid. MS 403.0 [M+H]⁺. Compounds 66 and 67 were synthesized in a similar manner as 61 by using the appropriately substituted aryl bromide variant.

Compound 66. 190 mg, 68%, a light yellow solid.

Compound 67. 175 mg, 52%, a light yellow solid.

Compound 64, 65, 68, 69, 72, 74, 76, 77, 78, 7879, 83, 84 and 85 were synthesized in a similar manner using appropriately substituted boronic acid and aryl bromide variants of reagents used to synthesize 61.

Compound 64. 260 mg, 43%, a white solid.

Compound 65. 290 mg, 65%, a white solid.

Compound 68. 35 mg, 29%, a yellow solid.

Compound 69. 45 mg, 35%, a yellow solid.

Compound 72. 93 mg, 44%, a white solid.

Compound 74. 158 mg, 49%, an off-white solid.

Compound 76. 70 mg, 25%, a light yellow solid.

Compound 77. 20 mg, 42%, an orange solid.

Compound 78. 65 mg, 29%, a white solid.

Compound 79. 23 mg, 41%, a white solid.

Compound 83. 80 mg, 36%, a light yellow solid.

Compound 84. 38 mg, 37%, a white solid.

Compound 85. 73 mg, 38%, a white solid.

### Example 19 Synthesis of Compound

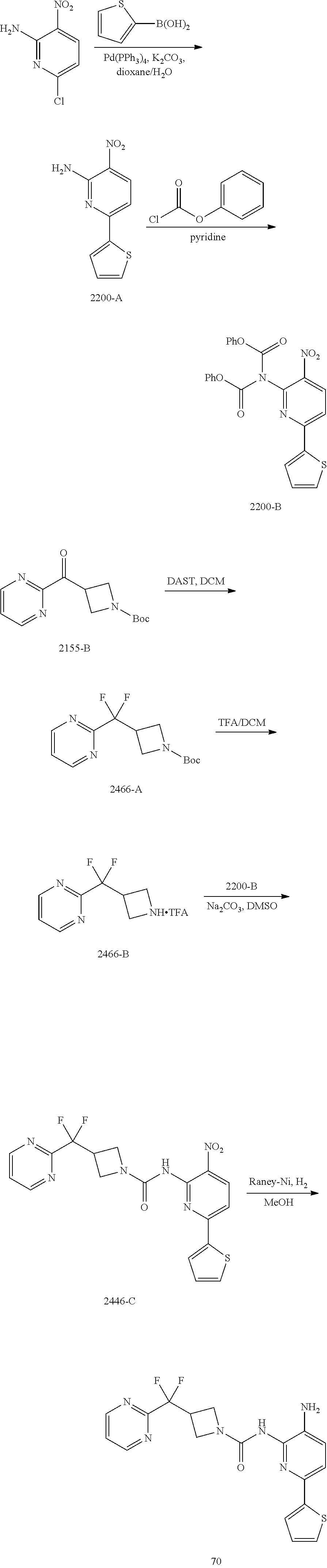

Synthesis of 2200-A. To a mixture of thiophen-2-ylboronic acid (14.1 g, 110 mmol), 6-chloro-3-nitropyridin-2-amine (17.3 g, 100 mmol) and K₂CO₃ (41.4 g, 300 mmol) in dioxane/H₂O (500 mL/50 mL) was added Pd(PPh₃)₄ (5.8 g, 5.0 mmol) under a nitrogen atmosphere. The reaction mixture was stirred at 100° C. for 2 h and then concentrated in vacuo. The residue was dissolved with EtOAc (200 mL) and the solution was washed with brine (100 mL×3). The organic layer was dried over anhydrous Na₂SO₄ and then concentrated in vacuo. The residue was purified by column chromatography on silica gel (PE:EtOAc=10:1 to 5:1) to give 2200-A (20.4 g, 84%) as a yellow solid. MS 222.0 [M+H]⁺.

Synthesis of 2200-B. To a stirred solution of 2200-A (4.42 g, 20 mmol) in pyridine (80 mL) was added phenyl carbonochloridate (3.12 g, 60 mmol) dropwise at 0° C. After the addition was completed, the mixture was stirred at 50° C. for 4 h. The mixture was then concentrated in vacuo, and the residue was purified by column chromatography on silica gel (PE:DCM=3:2 to 1:1) to give 2200-B (8.57 g, 93%) as a yellow solid. MS 462.1 [M+H]⁺.

Synthesis of 2466-A. To a solution of 2155-B (550 mg, 2.1 mmol) in DCM (10 mL) was added DAST (1.1 ml) dropwise at −78° C. under nitrogen atmosphere, and the reaction was allowed to slowly warm to room temperature and stirred at room temperature for 16 h. The solvent was concentrated and the residue was purified by Prep-TLC (EtOAc:PE=3:1) to give 2466-A (240 mg, 40%) as a brown solid. MS 286.2 [M+H]⁺.

Synthesis of 2466-B. A solution of 2466-A (240 mg, 0.84 mmol) in DCM (10 mL) was treated with TFA (4 mL) dropwise at 0° C. The solution was then warmed to room temperature and stirred at room temperature for 1 h, whereupon the solvent was removed in vacuo to give 2466-B as a crude product which was used directly in the next step.

Synthesis of 2466-C. To a mixture of 2200-B (260 mg, 0.56 mmol) and 2466-B (0.84 mmol, crude product from previous step) in DMSO (20 mL) was added Na₂CO₃ (285 mg, 0.88 mmol) and the reaction mixture was stirred at room temperature for 2 h. The mixture was then diluted with water (50 mL), extracted with EtOAc (50 mL×3), and the combined organic layers were washed with brine (50 mL×3), and then dried over anhydrous Na₂SO₄ and concentrated in vacuo. The residue was purified by Prep-TLC (EA:PE=5:1) to give 2466-C (150 mg, 62%) as a yellow solid. MS 433.0 [M+H]⁺.

Synthesis of 70 A mixture of 2466-C (150 mg, 0.35 mmol) and Raney-Ni (150 mg) in MeOH (8 mL) was stirred at room temperature for 1 h under a H₂ atmosphere. Raney-Ni was then removed by filtration through a pad of Celite, the filtrate was concentrated, and the residue was purified by Prep-TLC (EA:MeOH=15:1) to give 70 (84 mg, 59%) as yellow solid. MS 403.2 [M+H]⁺.

Compound 75 was synthesized in a similar manner as 70 using 2475-E instead of 2200-B

Compound 75. 275 mg, 74%, a light yellow solid.

### Example 20 Synthesis of Compound 71

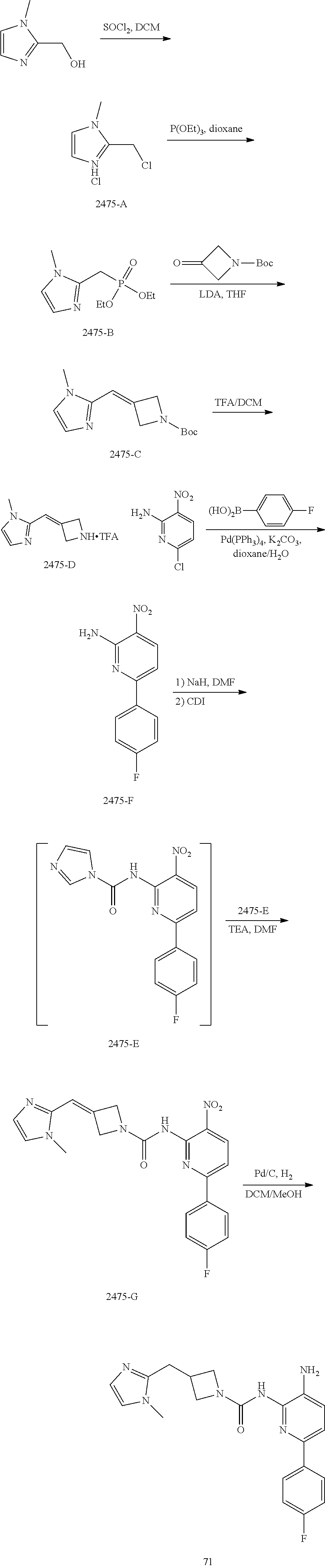

Synthesis of 2475-A. To a solution of (1-methyl-1H-imidazol-2-yl)methanol (4.5 g, 40.1 mmol) in DCM (90 mL) was added thionyl chloride (9 mL, 120.4 mmol) dropwise at 0° C. The reaction mixture stirred at room temperature for 4 h and then concentrated in vacuo to give 2475-A (5.95 g, 89%) as a white solid. MS 131.1 [M+1]⁺.

Synthesis of 2475-B. A stirred solution of 2475-A (3.0 g, 18.0 mmol) in dioxane (30 mL) was treated with triethyl phosphite (30 mL) under nitrogen atmosphere. The reaction mixture was stirred at 120° C. for 4 h and then concentrated in vacuo. The residue was purified by column chromatography on silica gel (EA:MeOH=100:1 to 10:1) to give 2475-B (960 mg, 23%) as a colorless oil. MS 233.2 [M+1]⁺.

Synthesis of 2475-C. To a solution of 2475-B (400 mg, 1.7 mmol) in THF (10 mL) was added LDA (2.6 mL, 5.2 mmol) dropwise at −78° C. under nitrogen atmosphere, and the reaction mixture was stirred for 1 h at −78° C. A solution of tert-butyl 3-oxoazetidine-1-carboxylate (441 mg, 2.6 mmol) in THF (5 mL) was then added dropwise to the mixture, while stirring at −78° C., and when the addition was completed the reaction was allowed to warm to room temperature and stirred for 16 h. The reaction mixture was then diluted with saturated NH₄Cl (40 mL), extracted with EtOAc (30 mL×3), and the combined organic layers were washed with brine (30 mL×3), dried over anhydrous Na₂SO₄ and then concentrated in vacuo. The residue was purified by column chromatography on silica gel (EtOAc) to give 2475-C (180 mg, 42%) as a white solid. MS 250.2 [M+H]⁺.

Synthesis of 2475-D. To a solution of 2475-C (180 mg, 0.72 mmol) in DCM (15 mL) was added TFA (3 mL) dropwise at 0° C. The reaction mixture was stirred at room temperature for 1 h, and then was concentrated in vacuo. The crude residue was dissolved in DMF (4 mL) and treated with TEA (218 mg, 2.16 mmol) to give 2475-D as a solution which was used directly in the next step. MS 150.2 [M+H]⁺.

Synthesis of 2475-F. A mixture of 6-chloro-3-nitropyridin-2-amine (4.58 g, 26.4 mmol), 4-fluorophenylboronic acid (4.44 g, 31.7 mmol) and K₂CO₃ (10.9 g, 79.2 mmol) in dioxane/H₂O (100 mL/10 mL) was added Pd(PPh₃)₄ (1.10 g, 0.95 mmol) under nitrogen atmosphere. The mixture was stirred at 100° C. for 2 h and then concentrated in vacuo. The residue was dissolved with EtOAc (200 mL) and the solution was washed with brine (100 mL×3). The organic layer was dried over anhydrous Na₂SO₄ and then concentrated in vacuo. The residue was purified by column chromatography on silica gel (PE:EtOAc=7:1 to 5:1) to give 2475-F (3.96 g, 64%) as a yellow solid. MS 234.2 [M+H]⁺.

Synthesis of 2475-G. To a solution of 2475-F (180 mg, 0.77 mmol) in DMF (5 mL) was added NaH (60% in mineral oil) (61 mg, 1.52 mmol) at ice bath and stirred at ice bath for 30 min, then CDI (133 mg, 0.84 mmol) was added into above mixture and stirred at ice bath for another 30 min. Finally, the solution of 2475-D was added into above mixture at ice bath and stirred at ice bath for 1 h. The mixture was quenched with water (40 mL) and extracted with EtOAc (40 mL×3). The combined organic layers were washed with brine (40 mL×3), dried over anhydrous Na₂SO₄ and then concentrated in vacuo. The residue was purified by column chromatography on silica gel (PE:EtOAc=2:1 to EtOAc) to give 2475-G (270 mg, 92%) as a yellow solid. MS 409.4 [M+H]⁺.

Synthesis of 71. A mixture of 2475-G (270 mg, 0.66 mmol) and Pd/C (270 mg) in MeOH/EtOAc (20 mL/20 mL) was stirred at room temperature for 1 h under a H₂ atmosphere. Pd/C was removed by filtration through a pad of Celite. The filtrate was concentrated in vacuo and the residue was purified by Prep-HPLC to give 71 (105 mg, 42%) as a yellow solid. MS 381.2 [M+H]⁺.

### Example 21 Synthesis of Compound 73

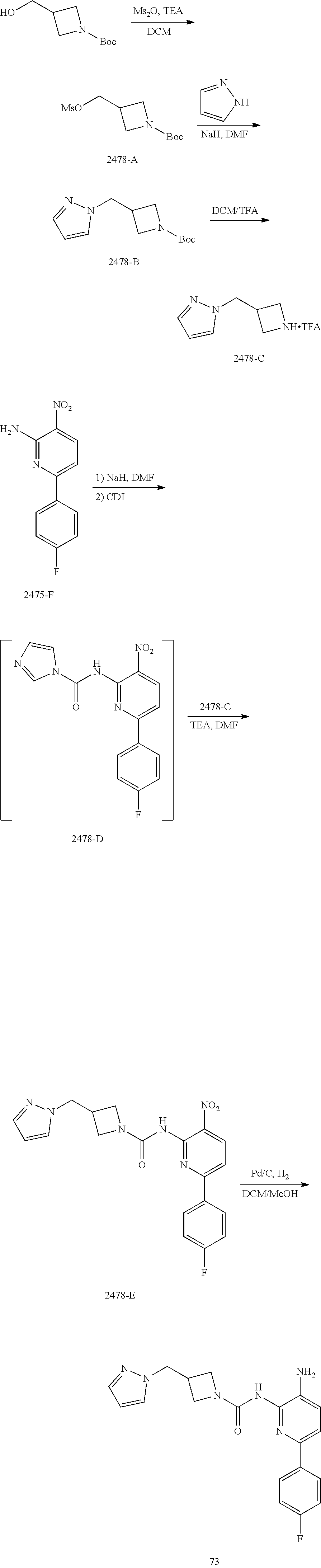

Synthesis of 2478-A. To a solution of tert-butyl 3-(hydroxymethyl)azetidine-1-carboxylate (1.12 g, 6.0 mmol) in DCM (30 mL) and triethylamine (1.82 g, 18.0 mmol) was added methanesulfonic anhydride (2.08 g, 12.0 mmol) dropwise at 0° C. The reaction mixture was stirred at room temperature for 16 h. The mixture was quenched with water (40 mL) and extracted with DCM (40 mL×3). The combined organic layers were washed with brine (40 mL×3), dried over anhydrous Na₂SO₄ and then concentrated in vacuo to give 2478-A (1.55 g, 97%) as a brown oil. MS 215.1 [M-55]⁺.

Synthesis of 2478-B. A solution of 1H-pyrazole (340 mg, 5 mmol) in DMF (10 mL) was cooled to 0° C. and then treated with NaH (60% in mineral oil) (400 mg, 10 mmol), and the reaction mixture was stirred 1 h at 0° C. Then a solution of 2478-A (1.33 g, 5 mmol) in DMF (3 mL) was added dropwise, and the resulting mixture was allowed to warm to room temperature and was stirred for 16 h at room temperature. The mixture was quenched with water (40 mL) and extracted with EtOAc (40 mL×3). The combined organic layers were washed with brine (40 mL×3), dried over anhydrous Na₂SO₄ and then concentrated in vacuo. The residue was purified by column chromatography on silica gel (PE:EtOAc=10:1 to 1:2) to give 2478-B (900 mg, 76%) as a colorless oil. MS 182.1 [M-55]⁺.

Synthesis of 2478-C. To a solution of 2478-B (237 mg, 1.0 mmol) in DCM (10 mL) was added TFA (3 mL) dropwise at 0° C. The reaction mixture was then allowed to warm to room temperature and was stirred at room temperature for 1 h. The solution was concentrated in vacuo, then the residue was dissolved in DMF (4 mL) and treated with TEA (303 mg, 3.0 mmol) to give 2478-C as a solution which was directly used in the next step. MS 138.2 [M+H]⁺.

Synthesis of 2478-E. A solution of 2475-F (233 mg, 1.0 mmol) in DMF (5 mL) was cooled to 0° C. and treated with NaH (60% in mineral oil) (80 mg, 2.0 mmol). The reaction mixture was stirred at 0° C. for 30 min, then CDI (180 mg, 1.1 mmol) was added into above mixture and stirring was continued at 0° C. for another 30 min. Finally, the solution of 2478-C was added, and the resulting reaction mixture was stirred at 0° C. for 1 h. The mixture was quenched with water (40 mL) and extracted with EtOAc (40 mL×3). The combined organic layers were washed with brine (40 mL×3), dried over anhydrous Na₂SO₄ and then concentrated in vacuo. The residue was purified by column chromatography (PE:EtOAc=4:1 to 1:1) to give 2478-E (350 mg, 88%) as a yellow solid. MS 397.4 [M+H]⁺.

Synthesis of 73. A mixture of 2478-E (350 mg, 0.88 mmol) and Pd/C (350 mg) in MeOH/EtOAc (20 mL/20 mL) was stirred at room temperature for 1 h under a H₂ atmosphere. Pd/C was removed by filtration through a pad of Celite. The filtrate was concentrated in vacuo and the residue was purified by Prep-TLC (EA:MeOH=10:1) to give 73 (200 mg, 62%) as a white solid. MS 367.1 [M+H]⁺.

### Example 22 Synthesis of Compound 80

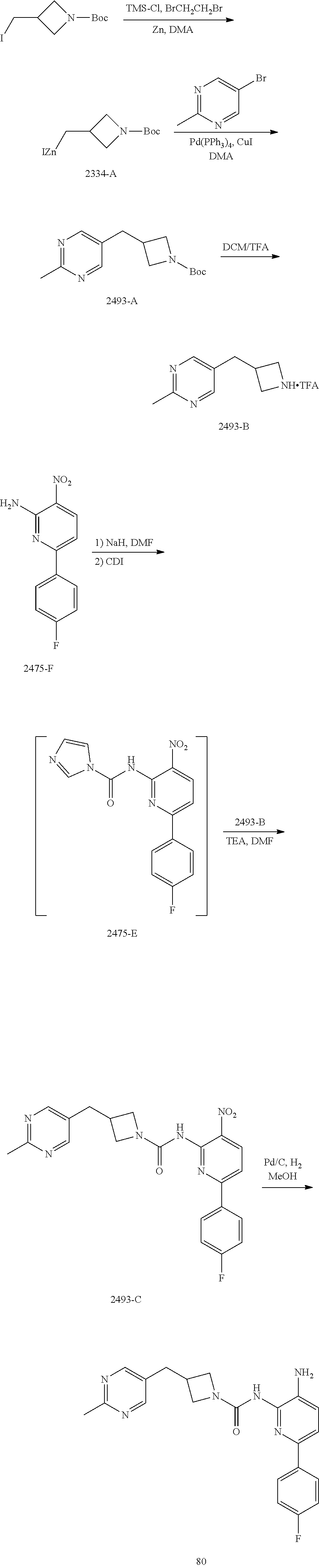

Synthesis of 2334-A. To a mixture of zinc dust (228 mg, 3.5 mmol) in anhydrous DMA (1 mL) were added TMSCl and 1,2-dibromoethane (0.06 mL, v/v=7/5). The resulting mixture was stirred at room temperature for 20 min under nitrogen atmosphere. Then a solution of tert-butyl 3-(iodomethyl)azetidine-1-carboxylate (800 mg, 2.7 mmol) in anhydrous DMA (1 mL) was added into above mixture. The resulting mixture continued to stir at room temperature for 16 h under nitrogen atmosphere. The mixture was used to next step directly as 2334-A. The concentration of 2334-A was about 1.0 mol/L in DMA.

Synthesis of 2493-A. To a mixture of 5-bromo-2-methylpyrimidine (344 mg, 2.0 mmol), CuI (38 mg, 0.2 mmol) and Pd(PPh₃)₄ (116 mg, 0.1 mmol) in anhydrous DMA (6 mL) under a nitrogen atmosphere was added 2334-A (2.0 mL). The resulting mixture was stirred at 60° C. for 48 h under a nitrogen atmosphere. The mixture was diluted with water (20 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=20:1 to 5:1) to give 2493-B (80 mg, 15%) as a yellow oil. MS 208.2 [M-55]⁺.

Synthesis of 2493-B. To a solution of 2493-A (80 mg, 0.3 mmol) in DCM (3 mL) was added TLA (1 mL) dropwise at 0° C. Then the solution was stirred at room temperature for 1 h. The solution was concentrated in vacuo. Then the residue was dissolved in DML (2 mL) and treated with TEA (91 mg, 0.9 mmol) to give 2493-B as a solution which was used directly in the next step. MS 164.1 [M+H]⁺.

Synthesis of 2493-C. A solution of 2475-F (71 mg, 0.3 mmol) in DML (2 mL) was cooled to 0° C. and treated with NaH (60% in mineral oil, 24 mg, 0.6 mmol). The reaction mixture was stirred at 0° C. for 30 min, then CDI (58 mg, 0.36 mmol) was added into above mixture and stirring continued at 0° C. for another 30 min. Finally, the solution of 2493-B was added, and the reaction mixture was stirred at 0° C. for 1 h. The mixture was quenched with water (10 mL) and extracted with EtOAc (10 mL×3). The combined organic layers were washed with brine (10 mL×3), dried over anhydrous Na₂SO₄ and then concentrated in vacuo. The residue was purified by Prep-TLC (DCM:EtOAc=1:1) to give 2493-C (85 mg, 67%) as a yellow solid. MS 423.1 [M+H]⁺.

Synthesis of 80. A mixture of 2493-D (85 mg, 0.2 mmol) and Pd/C (85 mg) in MeOH/EtOAc (3 mL/3 mL) was stirred at room temperature for 50 min under a H₂ atmosphere. Pd/C was removed by filtration through a pad of Celite. The filtrate was concentrated in vacuo and the residue was purified by Prep-HPLC to give 80 (230 mg, 63%) as a light yellow solid. MS 393.1 [M+H]⁺.

### Example 23 Synthesis of Compound 81

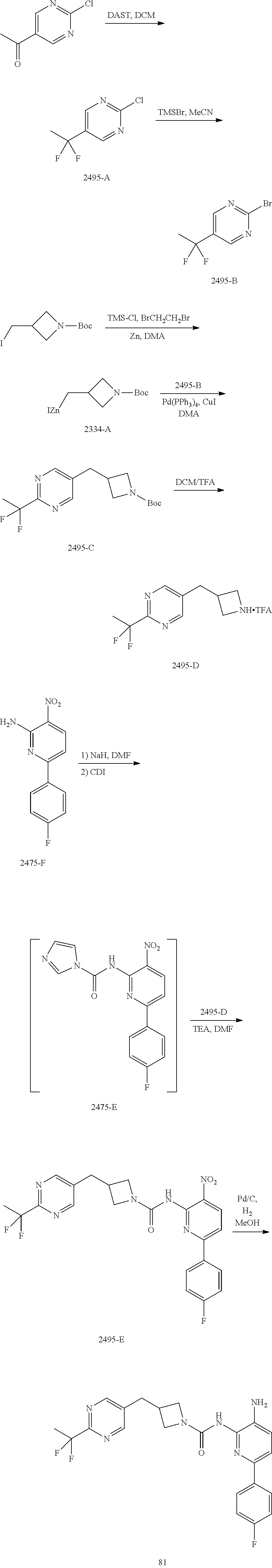

Synthesis of 2495-A. To a solution of l-(2-chloropyrimidin-5-yl)ethanone (1.8 g, 11.5 mmol) in DCM (50 mL) was added DAST (8.0 mL) dropwise at −78° C. under nitrogen atmosphere. Then the solution was warmed to room temperature for 16 h. The reaction was quenched with ice water (50 mL×3), extracted with DCM (30 mL×3). The combined organic layers were washed with brine (50 mL×3), dried over anhydrous Na₂SO₄ and then concentrated in vacuo. The residue was purified by column chromatography on silica gel (PE:EtOAc=20:1 to 8:1) to give 2495-A (1.4 g, 68%) as a yellow solid. MS 179.1, 181.1 [M+H]⁺.

Synthesis of 2495-B. A solution of 2495-A (700 mg, 4.0 mmol) and bromotrimethylsilane (1.84 g, 12.0 mmol) in acetonitrile (14 mL) was stirred at 75° C. for 16 h. The solvent was removed in vacuo. The residue was purified by column chromatography on silica gel (PE:EtOAc=10:1 to 5:1) to give 2495-B (500 mg, 56%) as a yellow solid. MS 223.0, 225.0 [M+H]⁺.

Synthesis of 2334-A. To a mixture of zinc dust (228 mg, 3.5 mmol) in anhydrous DMA (1 mL) was added TMSCl and 1,2-dibromoethane (0.06 mL, v/v=7/5) and stirred at room temperature for 20 min under nitrogen atmosphere. Then a solution of tert-butyl 3-(iodomethyl)azetidine-1-carboxylate (800 mg, 2.7 mmol) in anhydrous DMA (1 mL) was added into above mixture. The resulting mixture was stirred at room temperature for 16 h under nitrogen atmosphere. The mixture was used to next step directly as 2334-A. The concentration of 2334-A was about 1.0 mol/L in DMA.

Synthesis of 2495-C. To a mixture of 2495-B (444 mg, 2.0 mmol), CuI (38 mg, 0.2 mmol) and Pd(PPh₃)₄ (116 mg, 0.1 mmol) in anhydrous DMA (6 mL) under nitrogen atmosphere was added 2334-A (2.0 mL). The resulting mixture was stirred at 60° C. for 48 h under a nitrogen atmosphere. The mixture was diluted with water (20 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=20:1 to 5:1) to give 2495-B (330 mg, 53%) as a yellow oil. MS 258.2 [M-55]⁺.

Synthesis of 2495-D. To a solution of 2495-C (330 mg, 1.05 mmol) in DCM (9 mL) was added TLA (3 mL) drop wise at 0° C. Then the solution was stirred at room temperature for 1 h. The solution was concentrated in vacuo. Then the residue was dissolved in DML (5 mL) and treated with TEA (318 mg, 3.15 mmol) to give 2495-D as a solution which was directly used in the next step. MS 158.2 [M+H]⁺.

Synthesis of 2495-E. A solution of 2475-F (244 mg, 1.05 mmol) in DML (5 mL) was cooled to 0° C. and then treated with NaH (60% in mineral oil) (92 mg, 2.3 mmol). The reaction mixture was stirred at 0° C. for 30 min, then CDI (204 mg, 1.26 mmol) was added into above mixture and stirring was continued at 0° C. for another 30 min. Finally, the solution of 2495-D was added, and the reaction mixture was stirred at 0° C. for 1 h. The reaction was quenched with water (30 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (DCM:EtOAc=10:1 to 2:1) to give 2495-E (250 mg, 50%) as a yellow solid. MS 473.2 [M+H]⁺.

Synthesis of 81. A mixture of 2495-E (250 mg, 0.53 mmol) and Pd/C (250 mg) in MeOH/EtOAc (10 mL/10 mL) was stirred at room temperature for 50 min under a H₂ atmosphere. Pd/C was removed by filtration through a pad of Celite. The filtrate was concentrated in vacuo and the residue was purified by Prep-HPLC to give 81 (120 mg, 51%) as a off-white solid. MS 443.2 [M+H]⁺.

### Example 24 Synthesis of Compound 82

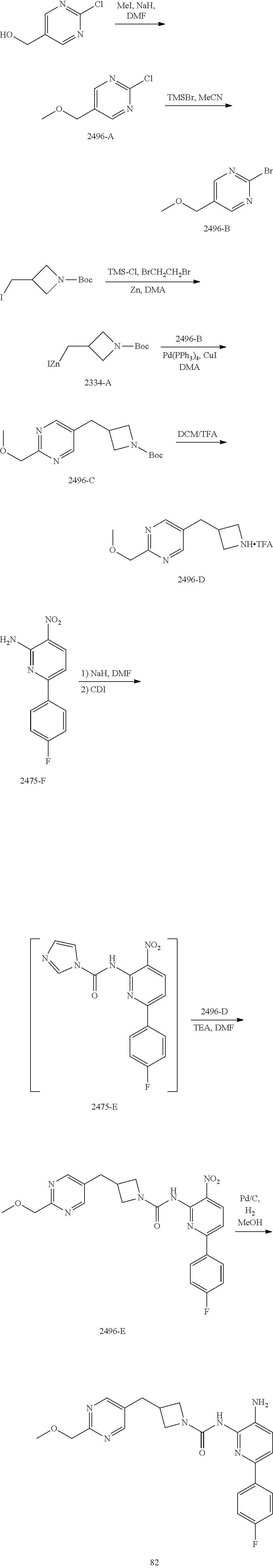

Synthesis of 2496-A. To a solution of (2-chloropyrimidin-5-yl)methanol (2.0 g, 13.9 mmol) and iodomethane (11.8 g, 83.4 mmol) in DMF (30 mL) was added NaH (60% in mineral oil, 583 mg, 14.6 mmol) at ice bath and then stirred at room temperature for 1 h. The mixture was diluted with water (90 mL) and extracted with EtOAc (40 mL×3). The combined organic layers were washed with brine (40 mL×3), dried over anhydrous Na₂SO₄ and then concentrated in vacuo. The residue was purified by column chromatography on silica gel (PE:EtOAc=20:1 to 10:1) to give 2496-A (1.4 g, 64%) as a yellow oil. MS 159.2, 161.2 [M+H]⁺. Synthesis of 2496-B. A solution of 2496-A (1.4 g, 8.9 mmol) and bromotrimethylsilane (4.1 g, 26.7 mmol) in acetonitrile (30 mL) was stirred at 75° C. 16 h. The solvent was removed in vacuo. The residue was purified by column chromatography on silica gel (PE:EtOAc=10:1 to 5:1) to give 2496-B (1.1 g, 61%) as a yellow solid. MS 203.1, 205.2 [M+H]⁺. Synthesis of 2334-A. To a mixture of zinc dust (228 mg, 3.5 mmol) in anhydrous DMA (1 mL) was added TMSCl and 1,2-dibromoethane (0.06 mL, v/v=7/5) and stirred at room temperature for 20 min under nitrogen atmosphere. Then a solution of tert-butyl 3-(iodomethyl)azetidine-1-carboxylate (800 mg, 2.7 mmol) in anhydrous DMA (1 mL) was added into above mixture. The resulting mixture was stirred at room temperature for 16 h under nitrogen atmosphere. The mixture was used to next step directly as 2334-A. The concentration of 2334-A was about 1.0 mol/L in DMA.

Synthesis of 2496-C. To a mixture of 2496-B (404 mg, 2.0 mmol), CuI (38 mg, 0.2 mmol) and Pd(PPh₃)₄ (116 mg, 0.1 mmol) in anhydrous DMA (6 mL) under nitrogen atmosphere was added 2334-A (2.0 mL). The resulting mixture was stirred at 60° C. for 48 h under nitrogen atmosphere. The mixture was diluted with water (20 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (PE:EtOAc=20:1 to 5:1) to give 2496-B (250 mg, 43%) as a yellow oil. MS 294.3 [M+H]⁺.

Synthesis of 2496-D. To a solution of 2495-C (250 mg, 0.85 mmol) in DCM (9 mL) was added TFA (3 mL) drop wise at 0° C. Then the solution was stirred at room temperature for 1 h. The solution was concentrated in vacuo. Then the residue was dissolved in DMF (5 mL) and treated with TEA (257.6 mg, 2.55 mmol) to give 2496-D as a solution which was directly used in the next step. MS 158.2 [M+H]⁺.

Synthesis of 2496-E. To a solution of 2475-F (198 mg, 0.85 mmol) in DMF (5 mL) was added NaH (60% in mineral oil, 68 mg, 1.7 mmol) at ice bath and the mixture was stirred at ice bath for 30 min, then CDI (165 mg, 1.02 mmol) was added into above mixture and stirred at ice bath for another 30 min. Finally, the solution of 2496-D was added into above mixture at ice bath and stirred at ice bath for 1 h. The mixture was quenched with water (30 mL) and extracted with EtOAc (20 mL×3). The combined organic layers were washed with brine (20 mL×3), dried over anhydrous Na₂SO₄ and then concentrated in vacuo. The residue was purified by column chromatography on silica gel (DCM:EtOAc=10:1 to 3:1) to give 2496-E (200 mg, 52%) as a yellow solid. MS 453.2 [M+H]⁺.

Synthesis of 82. A mixture of 2496-E (200 mg, 0.44 mmol) and Pd/C (200 mg) in MeOH/EtOAc (10 mL/10 mL) was stirred at room temperature for 50 min under H₂ atmosphere. Pd/C was removed by filtration through a pad of Celite. The filtrate was concentrated in vacuo and the residue was purified by Prep-TLC (DCM:MeOH=30:1) to give 82 (135 mg, 51%) as a off-white solid. MS 423.2 [M+H]⁺.

### Example 25 Synthesis of Compound 86

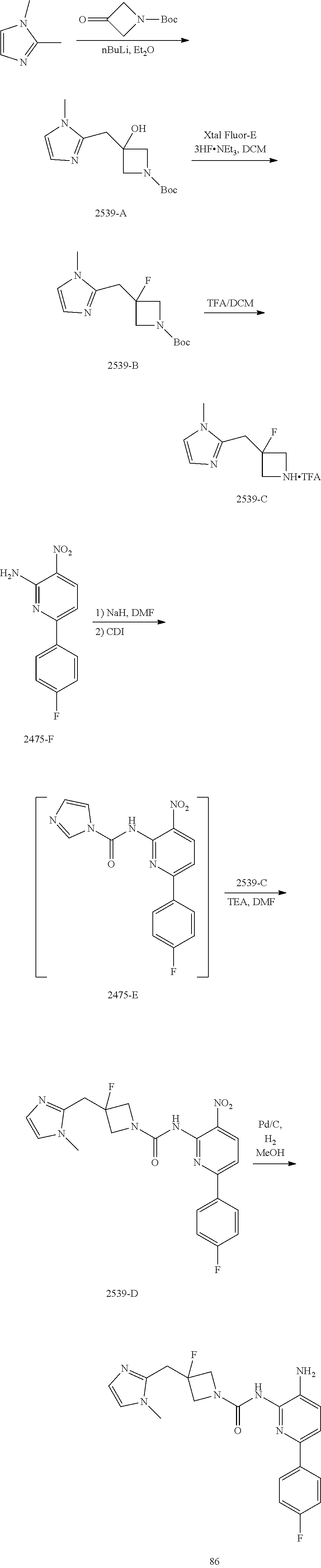

Synthesis of 2539-A. To a solution of 1,2-dimethyl-1H-imidazole (2.0 g, 20.8 mmol) in diethyl ether (40 mL) was added n-BuLi (25.0 mL, 62.4 mmol) dropwise at −78° C. and stirred at −78° C. for 1 h under nitrogen atmosphere. Then a solution of tert-butyl 3-oxoazetidine-1-carboxylate (10.7 g, 62.4 mmol) in diethyl ether (20 mL) was added into the above mixture dropwise at −78° C. The resulting mixture was warmed to room temperature for 3 h. The mixture was quenched with saturated NH₄Cl (40 mL), extracted with EtOAc (50 mL×3). The combined organic layers were washed with brine (50 mL×3), dried over anhydrous Na₂SO₄ and then concentrated in vacuo. The residue was purified by column chromatography on silica gel (PE:EtOAc=10:1 to EtOAc) to give 2539-A (2.0 g, 36%) as an off-white solid. MS 268.2 [M+H]⁺.

Synthesis of 2539-B. To a solution of 2539-A (800 mg, 3.0 mmol) in DCM (20 mL) was added XtalFluor-E (2.1 g, 9.0 mmol) and triethylamine trihydro fluoride (1.0 ml) dropwise at −78° C. under nitrogen atmosphere and then warmed to room temperature for 1 h. The mixture was quenched with saturated NaHCO₃ (50 mL), extracted with DCM (50 mL×3). The combined organic layers were washed with brine (50 mL×3), dried over anhydrous Na₂SO₄ and then concentrated in vacuo. The residue was purified by Prep-TLC (PE:EtOAc=1:3) to give 2539-B (500 mg, 62%) as a brown solid. MS 270.2 [M+H]⁺.

Synthesis of 2539-C. To a solution of 2539-B (500 mg, 1.86 mmol) in DCM (15 mL) was added TFA (5 mL) dropwise at 0° C. Then the solution was stirred at room temperature for 1 h. The solution was concentrated in vacuo. Then the residue was dissolved in DMF (6 mL) and treated with TEA (563 mg, 5.58 mmol) to give 2539-C as a solution which was directly used in the next step. MS 170.2 [M+H]⁺.

Synthesis of 2539-D. To a solution of 2475-F (440 mg, 1.89 mmol) in DMF (20 mL) was added NaH (60% in mineral oil) (113 mg, 2.83 mmol) at 0° C. and stirred for 30 min, then CDI (367 mg, 2.27 mmol) was added into above mixture and stirred at ice bath for another 30 min. Finally, the solution of 2539-C was added into above mixture at ice bath and stirred at ice bath for 1 h. The mixture was quenched with water (60 mL) and extracted with EtOAc (50 mL×3). The combined organic layers were washed with brine (30 mL×3), dried over anhydrous Na₂SO₄ and then concentrated in vacuo. The residue was purified by column chromatography on silica gel (PE:EtOAc=2:1 to EtOAc) to give 2539-D (700 mg, 87%) as a yellow solid. MS 429.0 [M+H]⁺.

Synthesis of 86. A mixture of 2539-D (700 mg, 1.64 mmol) and Pd/C (400 mg) in MeOH (10 mL) was stirred at room temperature for 1 h under H₂ atmosphere. Pd/C was removed by filtration through a pad of Celite. The filtrate was concentrated in vacuo and the residue was purified by Prep-TLC (EtOAc:MeOH=15:1) to give 86 (465 mg, 71%) as an off-white solid. MS 399.0 [M+H]⁺.

### Example 26 Synthesis of Compound 87

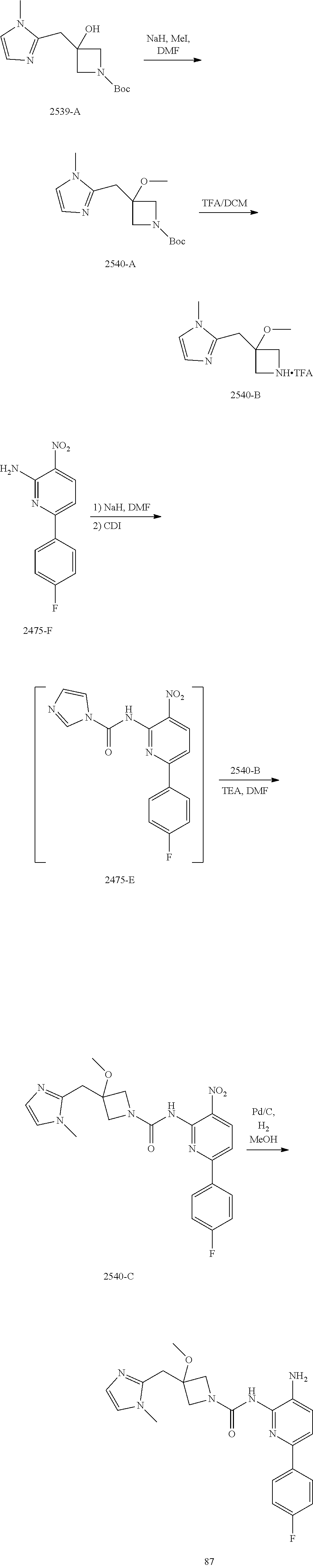

Synthesis of 2540-A. To a mixture of 2539-A (400 mg, 1.49 mmol) in DMF (20 mL) was added NaH (60% in mineral oil, 120 mg, 3.0 mmol) at room temperature and stirred at room temperature for 30 min. Then iodomethane (319 mg, 2.25 mmol) was added into above mixture dropwise. The resulting mixture was stirred at room temperature for 3 h. The solution was diluted with water (50 mL), extracted with EtOAc (50 mL×3). The combined organic layers were washed with brine (30 mL×3), dried over anhydrous Na₂SO₄ and then concentrated in vacuo to give 2540-A (400 mg, 96%) as an brown solid. MS 282.3 [M+H]⁺.

Synthesis of 2540-B. To a solution of 2540-A (400 mg, 1.42 mmol) in DCM (12 mL) was added TFA (4 mL) dropwise at 0° C. Then the solution was stirred at room temperature for 1 h. The solution was concentrated in vacuo. Then the residue was dissolved in DMF (6 mL) and treated with TEA (430 mg, 4.26 mmol) to give 2540-B as a solution which was directly used in the next step. MS 282.3 [M+H]⁺.

Synthesis of 2540-C. To a solution of 2475-F (350 mg, 1.5 mmol) in DMF (20 mL) was added NaH (60% in mineral oil, 90 mg, 2.3 mmol) at 0° C. and stirred at 0° C. for 30 min, then CDI (292 mg, 1.8 mmol) was added into above mixture and stirred at for another 30 min. Finally, the solution of 2540-B was added into above mixture at 0° C. and stirred for 1 h. The mixture was quenched with water (60 mL) and extracted with EtOAc (50 mL×3). The combined organic layers were washed with brine (30 mL×3), dried over anhydrous Na₂SO₄ and then concentrated in vacuo. The residue was purified by column chromatography on silica gel (PE:EtOAc=2:1 to EtOAc) to give 2540-D (350 mg, 53%) as a yellow solid. MS 441.0 [M+H]⁺.

Synthesis of 87. A mixture of 2540-D (350 mg, 0.79 mmol) and Pd/C (350 mg) in MeOH (10 mL) was stirred at room temperature for 1 h under a H₂ atmosphere. Pd/C was removed by filtration through a pad of Celite. The filtrate was concentrated in vacuo and the residue was purified by Prep-TLC (EtOAc:MeOH=15:1) to give 87 (220 mg, 68%) as an off-white solid. MS 411.2 [M+H]⁺.

TABLE 1
Exemplary Compounds and Spectrometric Data

| | | MS | MS | ¹H NMR Data (400 |
|---|---|---|---|---|
| No. | Structure | Calc. | found | MHz, DMSO-d₆) |
| 1 | 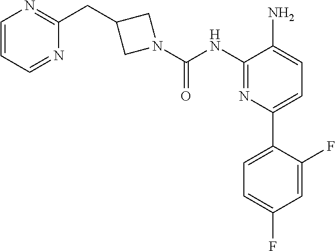 [SMILES: Nc1ccc(-c2ccc(F)cc2F)nc1NC(=O)N1CC(Cc2ncccn2)C1] | 396 | 397 | δ 8.75 (d, J = 4.9 Hz, 2H), 8.47 (s, 1H), 7.96- 7.90 (m, 1H), 7.38- 7.36 (m, 2H), 7.31- 7.26 (m, 2H), 7.18- 7.14 (m, 1H), 5.25 (s, 2H), 4.10 (t, J = 8.3 Hz, 2H), 3.78-3.75 (m, 2H), 3.25-2.23 (m, 2H), 3.13-3.05 (m, 1H). |
| 2 | 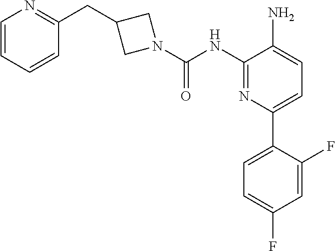 [SMILES: Nc1ccc(-c2ccc(F)cc2F)nc1NC(=O)N1CC(Cc2ccccn2)C1] | 395 | 396 | δ 8.50-8.47 (m, 2H), 7.96-7.90 (m, 1H), 7.74-7.69 (m, 1H), 7.37 (dd, J = 8.4, 2.4 Hz, 1H), 7.32-7.31 (m, 1H), 7.29-7.24 (m, 2H), 7.23-7.14 (m, 2H), 5.24 (s, 2H), 4.05 (t, J = 8.0 Hz, 2H), 3.76-3.73 (m, 2H), 3.08-2.98 (m, 3H). |
| 3 | 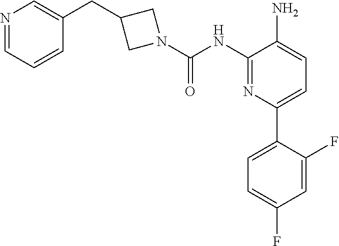 [SMILES: Nc1ccc(-c2ccc(F)cc2F)nc1NC(=O)N1CC(Cc2cccnc2)C1] | 395 | 396 | δ 8.49-8.44 (m, 2H), 8.43 (dd, J = 4.8, 1.6 Hz, 1H), 7.96-7.90 (m, 1H), 7.68-7.65 (m, 1H), 7.38-7.26 (m, 3H), 7.19-7.14 (m, 2H), 5.24 (s, 2H), 4.01 (t, J = 8.0 Hz, 2H), 3.72-3.68 (m, 2H), 2.94-2.86 (m, 3H). |
| 4 | 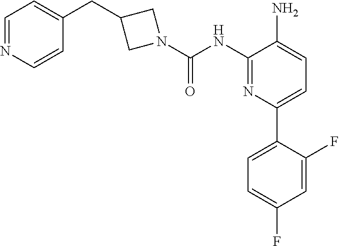 [SMILES: Nc1ccc(-c2ccc(F)cc2F)nc1NC(=O)N1CC(Cc2ccncc2)C1] | 395 | 396 | δ 8.49-8.47 (m, 3H), 7.96-7.89 (m, 1H), 7.37 (dd, J = 8.0, 5.6 Hz, 1H), 7.32-7.25 (m, 3H), 7.19-7.14 (m, 2H), 5.24 (s, 2H), 4.03 (t, J = 8.0 Hz, 2H), 3.71-3.67 (m, 2H), 2.95-2.90 (m, 3H). |
| 5 | 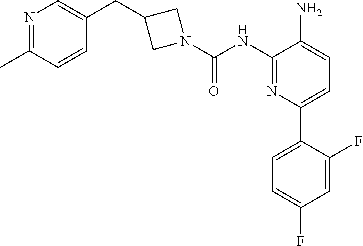 [SMILES: Cc1ccc(CC2CN(C(=O)Nc3nc(-c4ccc(F)cc4F)ccc3N)C2)cn1] | 409 | 410 | δ 8.47 (s, 1H), 8.32- 8.32 (m, 1H), 7.96- 7.89 (m, 1H), 7.53 (dd, J = 8.0, 2.0 Hz, 1H), 7.37 (dd, J = 8.0, 2.4 Hz, 1H), 7.32- 7.26 (m, 1H), 7.19- 7.14 (m, 3H), 5.24 (s, 2H), 3.99 (t, J = 8.0 Hz, 2H), 3.69-3.66 (m, 2H), 3.32-2.82 (m, 3H), 2.45 (s, 3H). |
| 6 | 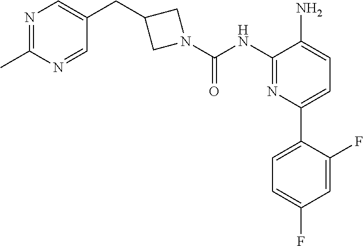 [SMILES: Cc1ncc(CC2CN(C(=O)Nc3nc(-c4ccc(F)cc4F)ccc3N)C2)cn1] | 410 | 411 | δ 8.60 (s, 2H), 8.49 (s, 1H), 7.93-7.92 (m, 1H), 7.38-7.14 (m, 4H), 5.24 (s, 2H), 4.01 (t, J = 7.2 Hz, 2H), 3.71-3.68 (m, 2H), 2.89 (s, 3H), 2.58 (s, 3H). |
| 7 | 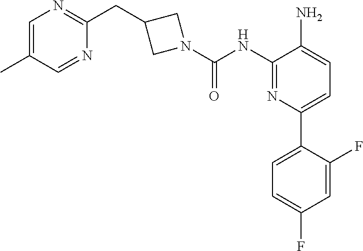 [SMILES: Cc1cnc(CC2CN(C(=O)Nc3nc(-c4ccc(F)cc4F)ccc3N)C2)nc1] | 410 | 411 | δ 8.60 (s, 2H), 8.49 (s, 1H),7.96-7.89 (m, 1H), 7.38-7.36 (m, 1H), 7.32-7.26 (m, 1H), 7.19-7.14 (m, 2H), 5.24 (s, 2H), 4.03- 3.99 (m, 2H), 3.71- 3.68 (m, 2H), 2.92- 2.87 (m, 3H), 2.58 (s, 3H). |
| 8 | 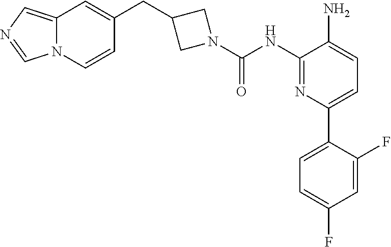 [SMILES: Nc1ccc(-c2ccc(F)cc2F)nc1NC(=O)N1CC(Cc2ccn3cncc3c2)C1] | 434 | 435 | δ 8.60 (s, 1H), 8.27 (d, J = 8.0 Hz, 2H), 7.96- 7.90 (m, 1H), 7.36 (dd, J = 8.0, 2.4 Hz, 1H), 7.31 (s, 1H), 7.29- 7.25 (m, 1H), 7.23 (s, 1H), 7.19-7.14 (m, 2H), 6.54 (dd, J = 7.2, 5.6 Hz, 1H), 5.24 (s, 2H), 4.04 (t, J = 8.0 Hz, 2H), 3.71-3.68 (m, 2H), 2.89-2.83 (m, 3H). |
| 9 | 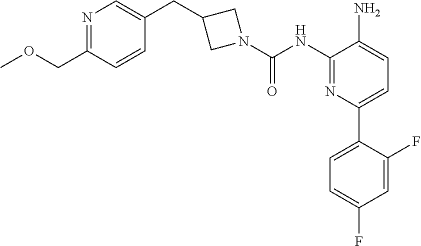 [SMILES: COCc1ccc(CC2CN(C(=O)Nc3nc(-c4ccc(F)cc4F)ccc3N)C2)cn1] | 439 | 440 | δ 8.48 (s, 1H), 8.41 (d, J = 1.6 Hz, 1H), 7.96- 7.90 (m, 1H), 7.70 (dd, J = 8.0, 6.4 Hz, 1H), 7.38-7.26 (m, 3H), 7.19-7.14 (m, 2H), 5.24 (s, 2H), 4.46 (s, 2H), 4.01 (t, J = 8.0 Hz, 2H), 3.71-3.68 (m, 2H), 3.35 (s, 3H), 2.93-2.85 (m, 3H). |
| 10 | 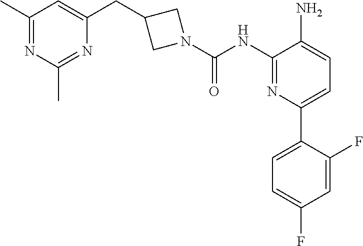 [SMILES: Cc1cc(CC2CN(C(=O)Nc3nc(-c4ccc(F)cc4F)ccc3N)C2)nc(C)n1] | 424 | 425 | δ 8.48 (s, 1H), 7.96- 7.90 (m, 1H), 7.37 (q, J = 2.4 Hz, 1H), 7.31-7.26 (m, 1H), 7.19-7.14 (m, 2H), 7.07 (s, 1H), 5.24 (s, 2H), 4.06 (t, J = 8.0 Hz, 2H), 3.73 (q, J = 4.4 Hz, 2H), 2.97 (s, 3H), 2.52 (s, 3H), 2.38 (s, 3H). |
| 11 | 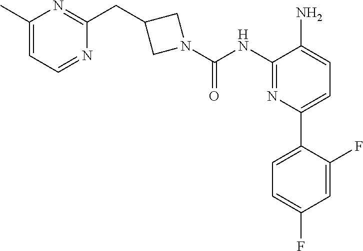 [SMILES: Cc1ccnc(CC2CN(C(=O)Nc3nc(-c4ccc(F)cc4F)ccc3N)C2)n1] | 410 | 411 | δ 8.54 (d, J = 5.2 Hz, 1H), 8.47 (s, 1H), 7.91- 7.89 (m, 1H), 7.35- 7.34 (m, 1H), 7.30- 7.20 (m, 2H), 7.16- 7.11 (m, 2H), 5.23 (s, 2H), 4.06 (t, J = 7.2 Hz, 2H), 3.73 (s, 2H), 3.15-3.14 (m, 2H), 3.04-3.03 (m, 1H), 2.40 (m, 3H). |
| 12 | 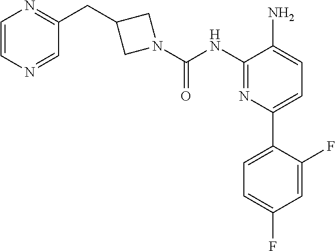 [SMILES: Nc1ccc(-c2ccc(F)cc2F)nc1NC(=O)N1CC(Cc2cnccn2)C1] | 396 | 397 | δ 8.57 (d, J = 11.6 Hz, 2H), 8.48 (s, 2H), 7.91- 7.89 (m, 1H), 7.35 (d, J = 7.2 Hz, 1H), 7.29-7.24 (m, 1H), 7.16-7.11 (m, 2H), 5.22 (s, 2H), 4.06- 4.01 (m, 2H), 3.74- 3.71 (m, 2H), 3.12- 3.11 (m, 2H), 3.00- 2.97 (m, 1H). |
| 13 | 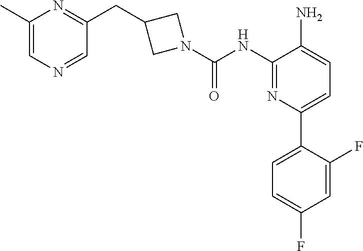 [SMILES: Cc1cncc(CC2CN(C(=O)Nc3nc(-c4ccc(F)cc4F)ccc3N)C2)n1] | 410 | 411 | δ 8.79 (s, 1H), 8.36 (d, J = 5.2 Hz, 2H), 7.90 (q, J = 8.4 Hz, 1H), 7.34 (d, J = 8.0 Hz, 1H), 7.27 (t, J = 9.6 Hz, 1H), 7.13 (t, J = 8.0 Hz, 2H), 5.22 (s, 2H), 4.03 (t, J = 8.0 Hz, 2H), 3.73 (t, J = 7.2 Hz, 2H), 3.05 (d, J = 7.6 Hz, 2H), 2.99- 2.95 (m, 1H), 2.40 (s, 3H). |
| 14 | 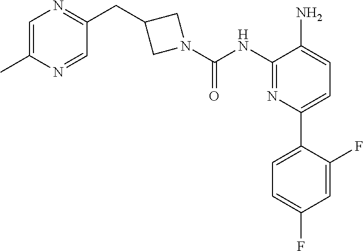 [SMILES: Cc1cnc(CC2CN(C(=O)Nc3nc(-c4ccc(F)cc4F)ccc3N)C2)cn1] | 410 | 411 | δ 8.46 (s, 1H), 8.43 (d, J = 2.0 Hz, 2H), 7.90 (q, J = 8.4 Hz, 1H), 7.35 (dd, J = 8.0 Hz, J = 2.0 Hz, 1H), 7.30- 7.24 (m, 1H), 7.17- 7.11 (m, 2H), 5.22 (s, 2H), 4.02 (t, J = 8.0 Hz, 2H), 3.73 (t, J = 5.2 Hz, 2H), 3.05 (d, J = 7.6 Hz, 2H), 2.97- 2.94 (m, 1H), 2.43 (s, 3H). |
| 15 | 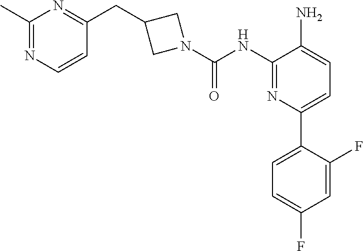 [SMILES: Cc1nccc(CC2CN(C(=O)Nc3nc(-c4ccc(F)cc4F)ccc3N)C2)n1] | 410 | 411 | δ 8.55 (d, J = 5.2 Hz, 1H), 8.48 (s, 1H), 7.90 (q, J = 8.0 Hz, 1H), 7.35 (d, J = 8.0 Hz, 1H), 7.27 (t, J = 8.8 Hz, 1H), 7.20-7.11 (m, 3H), 5.23 (s, 2H), 4.04 (t, J = 8.0 Hz, 2H), 3.72 (t, J = 5.2 Hz, 2H), 3.02-2.97 (m, 3H), 2.55 (s, 3H). |
| 16 | 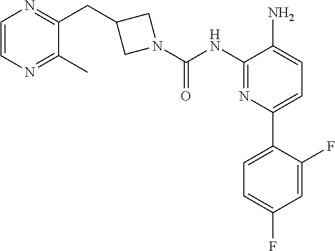 [SMILES: Cc1nccnc1CC1CN(C(=O)Nc2nc(-c3ccc(F)cc3F)ccc2N)C1] | 410 | 411 | CD₃OD as solvent. δ 8.35 (d, J = 2.4 Hz, 1H), 8.28 (d, J = 2.8 Hz, 1H), 7.91-7.85 (m, 1H), 7.42 (dd, J = 8.0, 2.4 Hz, 1H), 7.24 (d, J = 8.0 Hz, 1H), 7.03-6.97 (m, 2H), 4.31-4.27 (m, 2H), 3.89-3.86 (m, 2H), 3.26-3.24 (m, 3H), 2.58 (s, 3H). |
| 17 | 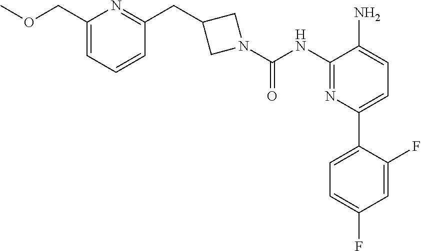 [SMILES: COCc1cccc(CC2CN(C(=O)Nc3nc(-c4ccc(F)cc4F)ccc3N)C2)n1] | 439 | 440 | δ 7.97 (q, J = 7.6 Hz, 1H), 7.67 (t, J = 7.6 Hz, 1H), 7.66 (d, J = 7.6 Hz, 1H), 7.24 (d, J = 7.6 Hz, 1H), 7.14 (q, J = 8.0 Hz, 2H), 7.06-6.98 (m, 3H), 4.73- 4.47 (m, 2H), 4.46 (s, 2H), 4.14-4.10 (m, 2H), 3.84-3.79 (m, 2H), 3.40 (s, 3H), 3.08- 3.02 (m, 3H). |
| 18 | 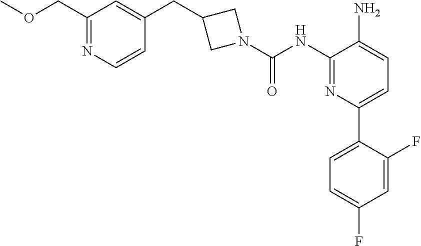 [SMILES: COCc1cc(CC2CN(C(=O)Nc3nc(-c4ccc(F)cc4F)ccc3N)C2)ccn1] | 439 | 440 | δ 8.41 (d, J = 4.8 Hz, 1H), 7.99-7.92 (m, 1H), 7.44 (d, J = 8.0 Hz, 1H), 7.26 (s, 1H), 7.16 (d, J = 8.0 Hz, 1H), 7.09-6.98 (m, 4H), 4.73 (s, 2H), 4.48 (s, 2H), 4.11-4.07 (m, 2H), 3.77-3.74 (m, 2H), 3.40 (s, 3H), 2.98 (s, 3H). |
| 19 | 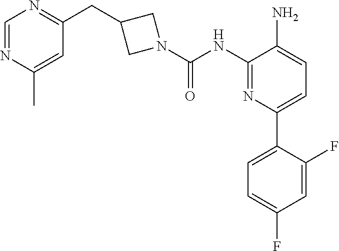 [SMILES: Cc1cc(CC2CN(C(=O)Nc3nc(-c4ccc(F)cc4F)ccc3N)C2)ncn1] | 410 | 411 | CD₃OD as solvent. δ 8.90 (d, J = 0.8 Hz, 1H), 7.89-7.85 (m, 1H), 7.42 (dd, J = 8.0, 2.0 Hz, 1H), 7.33 (s, 1H), 7.24 (d, J = 8.0 Hz, 1H), 7.04-6.97 (m, 2H), 4.22 (t, J = 8.0 Hz, 2H), 3.88- 3.85 (m, 2H), 3.14- 3.12 (m, 3H), 2.00 (s, 3H). |
| 20 | 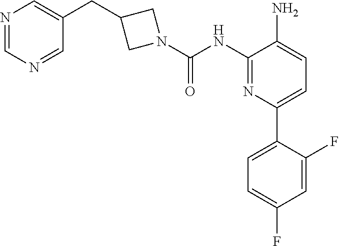 [SMILES: Nc1ccc(-c2ccc(F)cc2F)nc1NC(=O)N1CC(Cc2cncnc2)C1] | 396 | 397 | δ 9.06 (s, 1H), 8.73 (s, 2H), 8.50 (s, 1H), 7.96- 7.90 (m, 1H), 7.37 (dd, J = 8.0, 2.4 Hz, 1H), 7.32-7.26 (m, 1H), 7.19-7.14 (m, 2H), 5.24 (s, 2H), 4.03 (t, J = 8.0 Hz, 2H), 3.73-3.70 (m, 2H), 2.96-2.87 (m, 3H). |
| 21 | 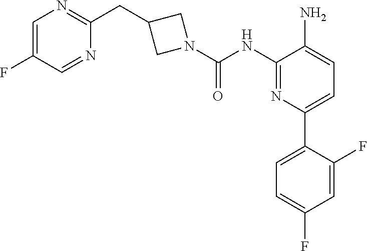 [SMILES: Nc1ccc(-c2ccc(F)cc2F)nc1NC(=O)N1CC(Cc2ncc(F)cn2)C1] | 414 | 415 | δ 8.81 (s, 2H), 8.46 (s, 1H), 7.90 (q, J = 8.8 Hz, 1H), 7.34 (q, J = 2.4 Hz, 1H), 7.29-7.33 (m, 1H), 7.13 (t, J = 8.4 Hz, 2H), 5.22 (s, 2H), 4.08 (t, J = 8.4 Hz, 2H), 3.74 (t, J = 6.0 Hz, 2H), 3.23 (d, J = 7.6 Hz, 2H), 3.05- 3.02 (m, 1H). |
| 22 | 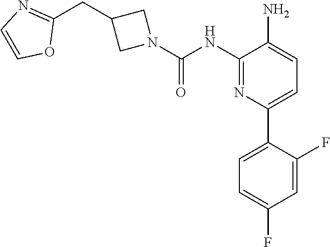 [SMILES: Nc1ccc(-c2ccc(F)cc2F)nc1NC(=O)N1CC(Cc2ncco2)C1] | 385 | 386 | δ 8.49 (s, 1H), 8.00 (d, J = 0.8 Hz, 1H), 7.93- 7.87 (m, 1H), 7.35 (dd, J = 8.4, 2.4 Hz, 1H), 7.29-7.23 (m, 1H), 7.16-7.10 (m, 3H), 5.23 (s, 2H), 4.08 (t, J = 8.0 Hz, 2H), 3.73-3.70 (m, 2H), 3.08 (d, J = 8.0 Hz, 2H), 2.97-2.94 (m, 1H). |
| 23 | 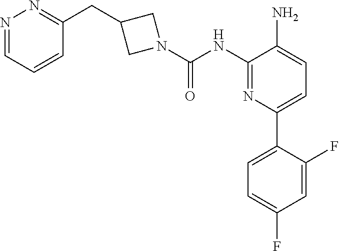 [SMILES: Nc1ccc(-c2ccc(F)cc2F)nc1NC(=O)N1CC(Cc2cccnn2)C1] | 396 | 397 | δ 9.11 (dd, J = 4.0, 2.0 Hz, 1H), 8.51 (s, 1H), 7.94-7.90 (m, 1H), 7.66-7.60 (m, 2H), 7.37 (dd, J = 8.0, 2.4 Hz, 1H), 7.32-7.26 (m, 1H), 7.19-7.14 (m, 2H), 5.25 (s, 2H), 4.08 (t, J = 8.0 Hz, 2H), 3.79-3.76 (m, 2H), 3.26 (d, J = 8.0 Hz, 2H), 3.17-3.04 (m, 1H). |
| 24 | 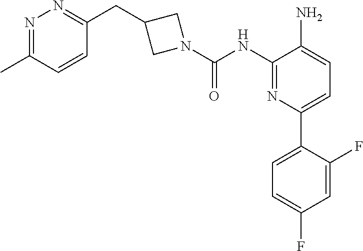 [SMILES: Cc1ccc(CC2CN(C(=O)Nc3nc(-c4ccc(F)cc4F)ccc3N)C2)nn1] | 410 | 411 | δ 8.49 (s, 1H), 7.96- 7.90 (m, 1H), 7.49 (s, 2H), 7.37 (dd, J = 8.0, 2.4 Hz, 1H), 7.31- 7.26 (m, 1H), 7.19- 7.14 (m, 2H), 5.24 (s, 2H), 4.06 (t, J = 8.0 Hz, 2H), 3.77-3.74 (m, 2H), 3.20 (d, J = 7.6 Hz, 2H), 3.06- 3.00 (m, 1H), 2.57 (s, 3H). |
| 25 | 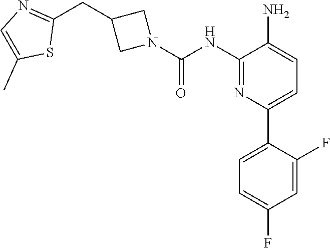 [SMILES: Cc1cnc(CC2CN(C(=O)Nc3nc(-c4ccc(F)cc4F)ccc3N)C2)s1] | 415 | 416 | δ 8.50 (s, 1H), 7.96- 7.89 (m, 1H), 7.38- 7.36 (m, 2H), 7.32- 7.26 (m, 1H), 7.19- 7.13 (m, 2H), 5.25 (s, 2H), 4.07 (t, J = 8.4 Hz, 2H), 3.76-3.73 (m, 2H), 3.23 (d, J = 7.6 Hz, 2H), 2.97- 2.93 (m, 1H), 2.40 (s, 3H). |
| 26 | 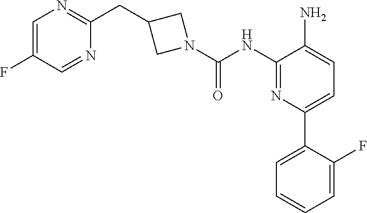 [SMILES: Nc1ccc(-c2ccccc2F)nc1NC(=O)N1CC(Cc2ncc(F)cn2)C1] | 396 | 397 | δ 8.83 (d, J = 0.8 Hz, 2H), 8.48 (s, 1H), 7.92- 7.87 (m, 1H), 7.41 (dd, J = 8.0, 2.0 Hz, 1H), 7.37-7.31 (m, 1H), 7.27-7.22 (m, 2H), 7.16-7.14 (m, 1H), 5.24 (s, 2H), 4.10 (t, J = 8.0 Hz, 2H), 3.78-3.74 (m, 2H), 3.26 (d, J = 7.2 Hz, 2H), 3.09-3.04 (m, 1H). |
| 27 | 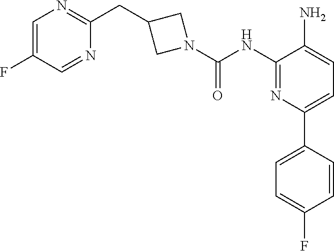 [SMILES: Nc1ccc(-c2ccc(F)cc2)nc1NC(=O)N1CC(Cc2ncc(F)cn2)C1] | 396 | 397 | δ 8.84 (s, 2H), 8.46 (s, 1H), 7.96 (dd, J = 8.8, 5.6 Hz, 2H), 7.53 (d, J = 8.4 Hz, 1H), 7.22 (t, J = 8.8 Hz, 2H), 7.14 (d, J = 8.0 Hz, 1H), 5.15 (s, 2H), 4.11 (t, J = 8.0 Hz, 2H), 3.78- 3.75 (m, 2H), 3.26 (d, J = 8.0 Hz, 2H), 3.10- 3.03 (m, 1H). |
| 28 | 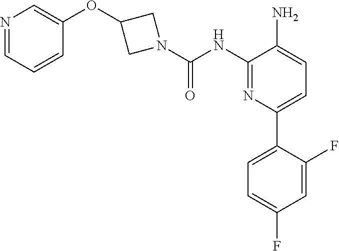 [SMILES: Nc1ccc(-c2ccc(F)cc2F)nc1NC(=O)N1CC(Oc2cccnc2)C1] | 397 | 398 | δ 8.60 (s, 1H), 8.26- 8.23 (m, 2H), 7.95- 7.89 (m, 1H), 7.40- 7.35 (m, 2H), 7.32- 7.26 (m, 2H), 7.18- 7.14 (m, 2H), 5.28 (s, 2H), 5.15-5.12 (m, 1H), 4.45 (dd, J = 9.2, 4.8 Hz, 2H), 3.96 (dd, J = 10.0, 4.0 Hz, 2H). |
| 29 | 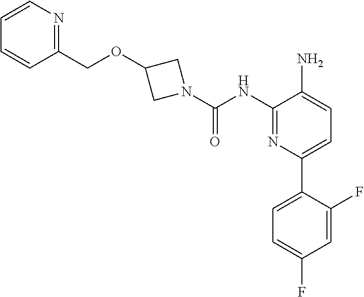 [SMILES: Nc1ccc(-c2ccc(F)cc2F)nc1NC(=O)N1CC(OCc2ccccn2)C1] | 411 | 412 | δ 8.61 (s, 1H), 8.50 (d, J = 5.6 Hz, 1H), 7.93- 7.87 (m, 1H), 7.82- 7.78 (m, 1H), 7.46 (d, J = 8.0 Hz, 1H), 7.37- 7.24 (m, 3H), 7.15- 7.11 (m, 2H), 5.22 (s, 2H), 4.53 (s, 2H), 4.46- 4.41 (m, 1H), 4.14 (dd, J = 9.2, 6.4 Hz, 2H), 3.82 (dd, J = 9.2, 4.0 Hz, 2H). |
| 30 | 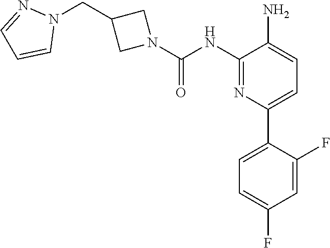 [SMILES: Nc1ccc(-c2ccc(F)cc2F)nc1NC(=O)N1CC(Cn2cccn2)C1] | 384 | 385 | δ 8.52 (s, 1H), 7.96- 7.89 (m, 1H), 7.77 (d, J = 2.0 Hz, 1H), 7.45 (d, J = 2.0 Hz, 1H), 7.37 (dd, J = 8.0, 5.6 Hz, 1H), 7.31-7.25 (m, 1H), 7.19-7.14 (m, 2H), 6.24 (t, J = 2.0 Hz, 1H), 5.23 (s, 2H), 4.37 (d, J = 8.0 Hz, 2H), 4.00 (t, J = 8.0 Hz, 2H), 3.80- 3.77 (m, 2H), 3.05- 2.99 (m, 1H). |
| 31 | 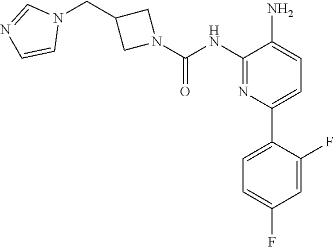 [SMILES: Nc1ccc(-c2ccc(F)cc2F)nc1NC(=O)N1CC(Cn2ccnc2)C1] | 384 | 385 | δ 8.54 (s, 1H), 7.95- 7.89 (m, 1H), 7.68 (s, 1H), 7.39-7.26 (m, 2H), 7.20-7.14 (m, 3H), 6.90 (s, 1H), 5.24 (s, 2H), 4.23 (d, J = 7.6 Hz, 2H), 4.00 (t, J = 8.4 Hz, 2H), 3.74- 3.71 (m, 2H), 2.99- 2.94 (m, 1H). |
| 32 | 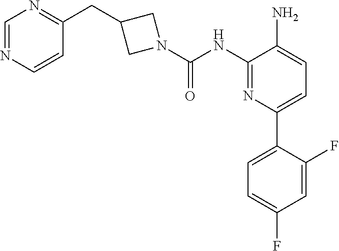 [SMILES: Nc1ccc(-c2ccc(F)cc2F)nc1NC(=O)N1CC(Cc2ccncn2)C1] | 396 | 397 | δ 9.09 (s, 1H), 8.70 (d, J = 5.2 Hz, 1H), 8.50 (s, 1H), 7.96-7.89 (m, 1H), 7.45 (q, J = 1.2 Hz, 1H), 7.37 (q, J = 2.4 Hz, 1H), 7.32- 7.26 (m, 1H), 7.19- 7.14 (m, 2H), 5.25 (s, 2H), 4.08 (t, J = 8.0 Hz, 2H), 3.75 (q, J = 6.0 Hz, 2H), 3.11- 3.02 (m, 3H). |
| 33 | 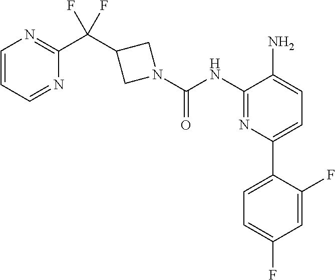 [SMILES: Nc1ccc(-c2ccc(F)cc2F)nc1NC(=O)N1CC(C(F)(F)c2ncccn2)C1] | 432 | 433 | δ 9.01 (d, J = 4.8 Hz, 2H), 8.69 (s, 1H), 7.96- 7.90 (m, 1H), 7.73 (t, J = 4.8 Hz, 1H), 7.38 (q, J = 2.4 Hz, 1H), 7.32-7.26 (m, 1H), 7.18-7.14 (m, 2H), 5.24 (s, 2H), 4.18 (t, J = 8.8 Hz, 2H), 4.06 (t, J = 5.6 Hz, 2H), 3.77-3.66 (m, 1H). |
| 34 | 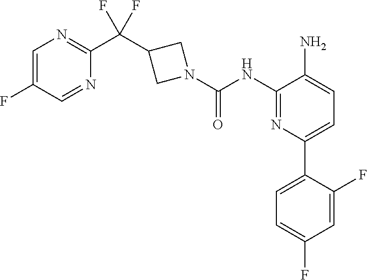 [SMILES: Nc1ccc(-c2ccc(F)cc2F)nc1NC(=O)N1CC(C(F)(F)c2ncc(F)cn2)C1] | 450 | 451 | δ 9.10 (s, 2H), 8.69 (s, 1H), 7.96-7.89 (m, 1H), 7.38 (q, J = 2.0 Hz, 1H), 7.32- 7.26 (m, 1H), 7.18- 7.13 (m, 2H), 5.24 (s, 2H), 4.18 (t, J = 9.2 Hz, 2H), 4.04 (q, J = 5.6 Hz, 2H), 3.74- 3.69 (m, 1H). |
| 35 | 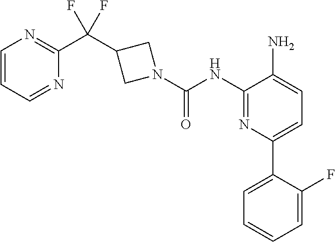 [SMILES: Nc1ccc(-c2ccccc2F)nc1NC(=O)N1CC(C(F)(F)c2ncccn2)C1] | 414 | 415 | δ 9.01 (d, J = 4.8 Hz, 2H), 8.68 (s, 1H), 7.92- 7.87 (m, 1H), 7.72 (t, J = 4.8 Hz, 1H), 7.42 (q, J = 2.0 Hz, 1H), 7.37-7.32 (m, 1H), 7.27-7.22 (m, 2H), 7.16 (d, J = 8.0 Hz, 1H), 5.23 (s, 2H), 4.18 (t, J = 9.2 Hz, 2H), 4.06 (q, J = 5.6 Hz, 2H), 3.76-3.68 (m, 1H). |
| 36 | 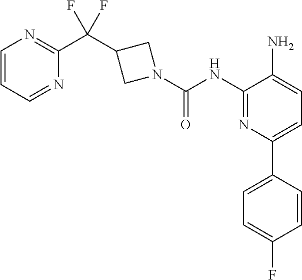 [SMILES: Nc1ccc(-c2ccc(F)cc2)nc1NC(=O)N1CC(C(F)(F)c2ncccn2)C1] | 414 | 415 | δ 9.01 (d, J = 4.8 Hz, 2H), 8.66 (s, 1H), 7.98- 7.95 (m, 2H), 7.73 (t, J = 4.8 Hz, 1H), 7.54 (d, J = 8.4 Hz, 1H), 7.22 (t, J = 8.8 Hz, 2H), 7.16 (d, J = 8.4 Hz, 1H), 5.14 (s, 2H), 4.18 (t, J = 8.8 Hz, 2H), 4.06 (q, J = 5.6 Hz, 2H), 3.76- 3.67 (m, 1H). |
| 37 | 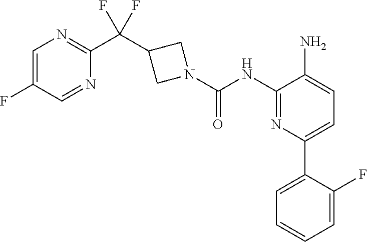 [SMILES: Nc1ccc(-c2ccccc2F)nc1NC(=O)N1CC(C(F)(F)c2ncc(F)cn2)C1] | 432 | 433 | δ 9.10 (s, 2H), 8.69 (s, 1H), 7.92-7.87 (m, 1H), 7.42 (q, J = 2.4 Hz, 1H), 7.35- 7.32 (m, 1H), 7.27- 7.22 (m, 2H), 7.16 (d, J = 8.0 Hz, 1H), 5.23 (s, 2H), 4.18 (t, J = 8.8 Hz, 2H), 4.05 (t, J = 5.2 Hz, 2H), 3.80- 3.66 (m, 1H). |
| 38 | 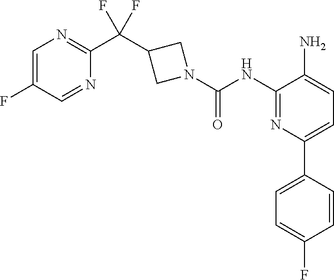 [SMILES: Nc1ccc(-c2ccc(F)cc2)nc1NC(=O)N1CC(C(F)(F)c2ncc(F)cn2)C1] | 432 | 433 | δ 9.10 (s, 2H), 8.67 (s, 1H), 7.98-7.95 (m, 2H), 7.54 (d, J = 8.4 Hz, 1H), 7.22 (q, J = 1.6 Hz, 2H), 7.16 (d, J = 8.4 Hz, 1H), 5.14 (s, 2H), 4.18 (t, J = 9.2 Hz, 2H), 4.05 (t, J = 5.6 Hz, 2H), 3.75- 3.67 (m, 1H). |
| 39 | 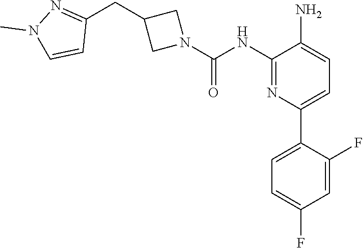 [SMILES: Cn1ccc(CC2CN(C(=O)Nc3nc(-c4ccc(F)cc4F)ccc3N)C2)n1] | 432 | 433 | δ 8.46 (s, 1H), 7.96- 7.90 (m, 1H), 7.55 (d, J = 2.0 Hz, 1H), 7.37 (dd, J = 8.0 Hz, 2.0 Hz, 1H), 7.32-7.26 (m, 1H), 7.19-7.14 (m, 2H), 6.02 (d, J = 2.4 Hz, 1H), 5.24 (s, 2H), 4.05-4.01 (m, 2H), 3.76 (s, 3H), 3.68- 3.66 (m, 2H), 3.76 (s, 3H). |
| 40 | 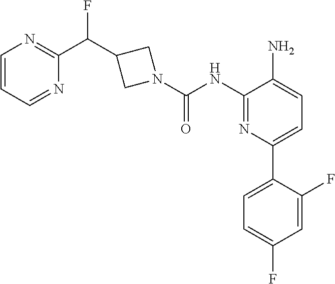 [SMILES: Nc1ccc(-c2ccc(F)cc2F)nc1NC(=O)N1CC(C(F)c2ncccn2)C1] | 414 | 415 | δ 8.90 (d, J = 4.8 Hz, 2H), 8.58 (s, 1H), 7.96- 7.90 (m, 1H), 7.56 (t, J = 4.8 Hz, 1H), 7.38 (q, J = 2.0 Hz, 1H), 7.32-7.26 (m, 1H), 7.18-7.14 (m, 2H), 5.84 (dd, J = 47.6 Hz, J = 6.4 Hz, 1H), 5.24 (s, 2H), 4.13- 4.03 (m, 3H), 3.91 (q, J = 6.0 Hz, 1H), 3.33- 3.26 (m, 1H). |
| 41 | 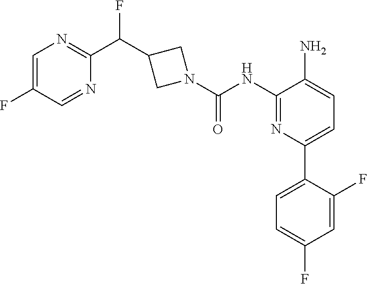 [SMILES: Nc1ccc(-c2ccc(F)cc2F)nc1NC(=O)N1CC(C(F)c2ncc(F)cn2)C1] | 432 | 433 | δ 9.00 (s, 2H), 8.58 (s, 1H), 7.96-7.90 (m, 1H), 7.38 (q, J = 2.0 Hz, 1H), 7.32-7.26 (m, 1H), 7.18-7.14 (m, 2H), 5.89 (dd, J = 47.6 Hz, 6.4 Hz, 1H), 5.24 (s, 2H), 4.10- 4.02 (m, 3H), 3.90 (t, J = 6.0 Hz, 1H), 3.33- 3.26 (m, 1H). |
| 42 | 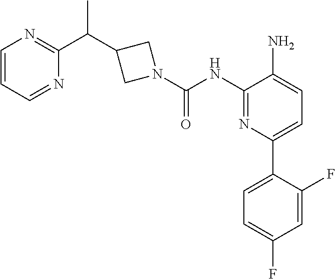 [SMILES: CC(c1ncccn1)C1CN(C(=O)Nc2nc(-c3ccc(F)cc3F)ccc2N)C1] | 410 | 411 | δ 8.76 (d, J = 4.8 Hz, 2H), 8.45 (s, 1H), 7.95- 7.89 (m, 1H), 7.38- 7.35 (m, 2H), 7.31- 7.25 (m, 1H), 7.18- 7.13 (m, 2H), 5.24 (s, 2H), 4.11 (t, J = 8.0 Hz, 1H), 3.93-3.81 (m, 2H), 3.65 (q, J = 5.6 Hz, 1H), 3.29- 3.24 (m, 1H), 2.94- 2.91 (m, 1H), 1.24 (d, J = 6.8 Hz, 3H). |
| 43 | 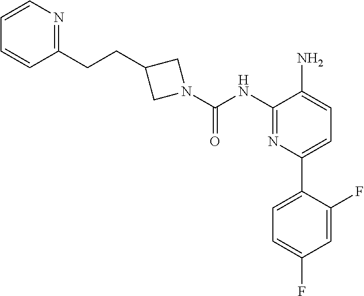 [SMILES: Nc1ccc(-c2ccc(F)cc2F)nc1NC(=O)N1CC(CCc2ccccn2)C1] | 409 | 410 | δ 8.51-8.46 (m, 2H), 7.96-7.89 (m, 1H), 7.72-7.68 (m, 1H), 7.37 (dd, J = 8.4, 2.4 Hz, 1H), 7.32-7.26 (m, 2H), 7.22-7.14 (m, 3H), 5.24 (s, 2H), 4.02-3.98 (m, 2H), 3.59-3.55 (m, 2H), 2.72-2.67 (m, 2H), 2.57-2.55 (m, 1H), 2.00-1.94 (m, 2H). |
| 44 | 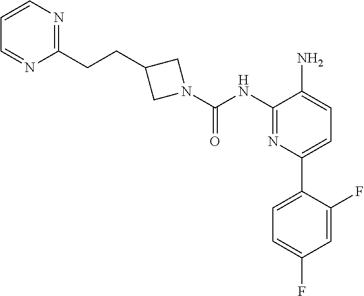 [SMILES: Nc1ccc(-c2ccc(F)cc2F)nc1NC(=O)N1CC(CCc2ncccn2)C1] | 410 | 411 | δ 8.73 (d, J = 5.2 Hz, 2H), 8.46 (s, 1H), 7.96- 7.89 (m, 1H), 7.38- 7.34 (m, 2H), 7.31- 7.26 (m, 1H), 7.18- 7.14 (m, 2H), 5.23 (s, 2H), 4.00 (t, J = 8.4 Hz, 2H), 3.57 (t, J = 6.0 Hz, 2H), 2.85 (t, J = 7.6 Hz, 2H), 2.59- 2.55 (m, 1H), 2.09- 2.03 (m, 2H). |
| 45 | 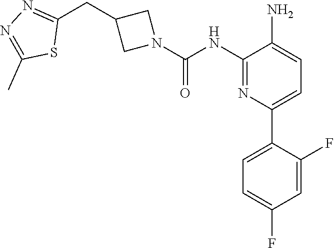 [SMILES: Cc1nnc(CC2CN(C(=O)Nc3nc(-c4ccc(F)cc4F)ccc3N)C2)s1] | 416 | 417 | δ 8.52 (s, 1H), 8.32 (s, 1H), 7.89-7.96 (m, 1H), 7.34-7.38 (m, 1H), 7.25-7.31 (m, 1H), 7.14-7.19 (m, 2H), 5.24 (s, 2H), 4.08 (t, J = 8.0 Hz, 2H), 3.75 (m, 2H), 3.38 (d, J = 8.0 Hz, 2H), 2.96- 3.01 (m, 1H), 2.68 (s, 3H). |
| 46 | 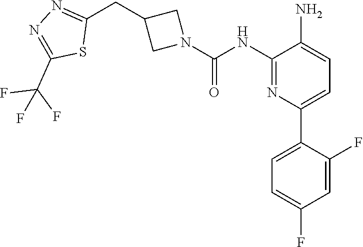 [SMILES: Nc1ccc(-c2ccc(F)cc2F)nc1NC(=O)N1CC(Cc2nnc(C(F)(F)F)s2)C1] | 470 | 471 | δ 8.54 (s, 1H), 7.96- 7.90 (m, 1H), 7.37 (dd, J = 8.0, 2.4 Hz, 1H) 7.32-7.26 (m, 1H), 7.19-7.14 (m, 2H), 5.24 (d, J = 6.0 Hz, 2H), 4.12 (t, J = 8.4 Hz, 2H), 3.82- 3.78 (m, 2H), 3.61 (d, J = 7.6 Hz, 2H), 3.12- 3.05 (m, 1H). |
| 47 | 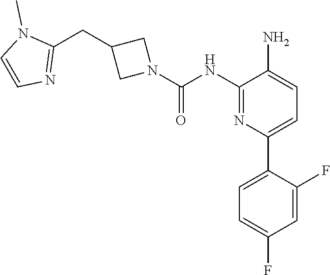 [SMILES: Cn1ccnc1CC1CN(C(=O)Nc2nc(-c3ccc(F)cc3F)ccc2N)C1] | 398 | 399 | δ 8.47 (s, 1H), 7.96- 7.90 (m, 1H), 7.37 (dd, J = 8.0, 2.4 Hz, 1H), 7.31-7.26 (m, 1H), 7.18-7.14 (m, 2H), 7.02 (d, J = 1.2 Hz, 1H), 6.73 (d, J = 1.2 Hz, 1H), 5.25 (s, 2H), 4.09 (t, J = 0.8 Hz, 2H), 3.73-3.70 (m, 2H), 3.56-3.54 (m, 3H), 2.99-2.93 (m, 3H). |
| 48 | 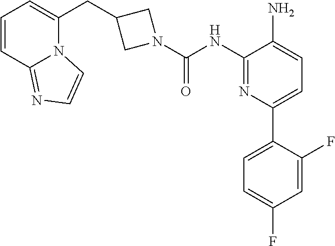 [SMILES: Nc1ccc(-c2ccc(F)cc2F)nc1NC(=O)N1CC(Cc2cccc3nccn23)C1] | 434 | 435 | δ 8.53 (s, 1H), 8.00 (s, 1H), 7.96-7.90 (m, 1H), 7.65 (s, 1H), 7.50 (d, J = 9.2 Hz, 1H), 7.37 (dd, J = 8.0, 2.0 Hz, 1H), 7.39-7.23 (m, 2H), 7.21-7.14 (m, 2H), 6.71 (d, J = 5.6 Hz, 1H), 5.25 (s, 2H), 4.15 (t, J = 8.0 Hz, 2H), 3.79-3.75 (m, 2H), 3.30-2.85 (m, 2H), 3.17-3.10 (m, 1 H). |
| 49 | 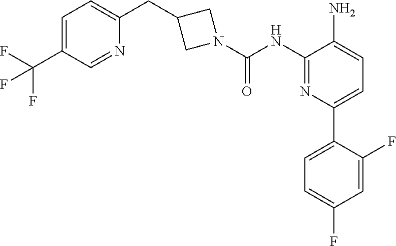 [SMILES: Nc1ccc(-c2ccc(F)cc2F)nc1NC(=O)N1CC(Cc2ccc(C(F)(F)F)cn2)C1] | 463 | 464 | δ 8.89 (s, 1H), 8.49 (s, 1H), 8.15 (t, J = 5.6 Hz, 2H), 7.93 (q, J = 2.0 Hz, 1H), 7.55 (d, J = 8.4 Hz, 1H), 7.38 (t, J = 2.8 Hz, 1H), 7.19- 7.14 (m, 2H), 5.24 (s, 2H), 4.07 (t, J = 7.6 Hz, 2H), 3.76 (s, 2H), 3.21 (d, J = 6.0 Hz, 2H), 3.04 (s, 1H). |
| 50 | 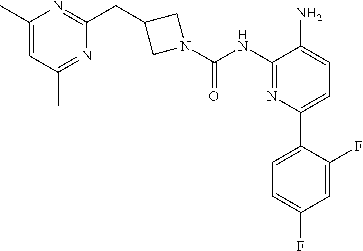 [SMILES: Cc1cc(C)nc(CC2CN(C(=O)Nc3nc(-c4ccc(F)cc4F)ccc3N)C2)n1] | 424 | 425 | δ 8.45 (s, 1H), 7.90 (q, J = 8.4 Hz, 1H), 7.34 (d, J = 7.6 Hz, 1H), 7.26 (t, J = 10.0 Hz, 1H), 7.13 (t, J = 8.4 Hz, 2H), 7.07 (s, 1H), 5.23 (s, 2H), 4.05 (t, J = 8.4 Hz, 2H), 3.73 (t, J = 6.4 Hz, 2H), 3.06- 3.01 (m, 3H), 2.35 (s, 6H). |
| 51 | 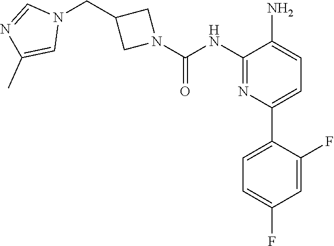 [SMILES: Cc1cn(CC2CN(C(=O)Nc3nc(-c4ccc(F)cc4F)ccc3N)C2)cn1] | 398 | 399 | δ 8.52 (s, 1H), 7.93- 7.87 (m, 1 H), 7.50 (s, 1H), 7.37-7.35 (m, 1H), 7.30-7.24 (m, 1H), 7.16-7.12 (m, 2H), 6.85 (s, 1H), 5.22 (s, 2H), 4.13-4.11 (d, J = 7.6 Hz, 2H), 3.97- 3.95 (m, 2 H), 3.70- 3.67 (m, 2 H), 2.93- 2.88 (m, 1H), 2.04 (s, 3H). |
| 52 | 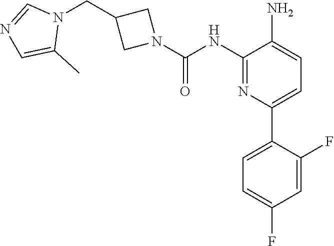 [SMILES: Cc1cncn1CC1CN(C(=O)Nc2nc(-c3ccc(F)cc3F)ccc2N)C1] | 398 | 399 | δ 8.58 (s, 1H), 7.96- 7.90 (m, 1H), 7.56 (s, 1H), 7.37 (d, J = 8.0 Hz, 1H), 7.32-7.27 (m, 1H), 7.19-7.15 (m, 2H), 6.63 (s, 1H), 5.26 (s, 2H), 4.14 (d, J = 7.6 Hz, 2H), 4.00 (t, J = 8.4 Hz, 2H), 3.75- 3.71 (m, 2H), 3.0- 2.92 (m, 1H), 2.17 (s, 3H). |
| 53 | 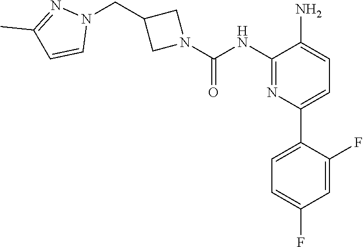 [SMILES: Cc1ccn(CC2CN(C(=O)Nc3nc(-c4ccc(F)cc4F)ccc3N)C2)n1] | 398 | 399 | δ 8.52 (s, 1H), 7.96- 7.90 (m, 1H), 7.62 (d, J = 1.0 Hz, 1H), 7.38- 7.25 (m, 2H), 7.19- 7.14 (m, 2H), 6.00 (d, J = 2.0 Hz, 1H), 5.23 (s, 2H), 4.26 (d, J = 7.2 Hz, 2H), 4.00 (t, J = 8.4 Hz, 2H), 3.79- 3.75 (m, 2H), 3.02- 2.95 (m, 1H), 2.15 (s, 3H). |
| 54 | 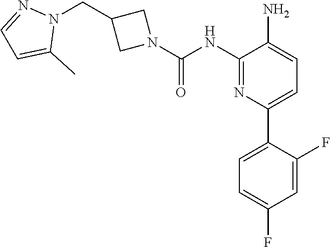 [SMILES: Cc1ccnn1CC1CN(C(=O)Nc2nc(-c3ccc(F)cc3F)ccc2N)C1] | 398 | 399 | δ 8.51 (s, 1H), 7.96- 7.90 (m, 1H), 7.40- 7.36 (m, 1H), 7.30- 7.25 (m, 2H), 7.4- 7.14 (m, 2H), 6.02 (s, 1H), 5.24 (s, 2H), 4.26 (d, J = 7.6 Hz, 2H), 4.00 (t, J = 8.4 Hz, 2H), 3.83-3.79 (m, 2H), 3.05-2.98 (m, 1H), 2.28 (s, 3H). |
| 55 | 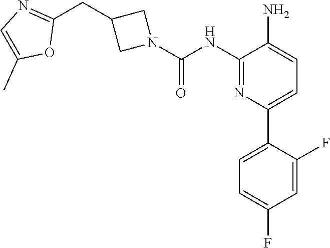 [SMILES: Cc1cnc(CC2CN(C(=O)Nc3nc(-c4ccc(F)cc4F)ccc3N)C2)o1] | 399 | 400 | δ 8.48 (s, 1H), 7.94- 7.87 (m, 1H), 7.35 (dd, J = 8, 2.4 Hz, 1H), 7.29-7.23 (m, 1H), 7.16-7.11 (m, 2H), , 6.68 (d, J = 1.2 Hz, 1H), 5.22 (s, 2H), 4.09-4.05 (m, 2H), 3.72-3.69 (m, 2H), 3.00 (d, J = 7.6 Hz, 2H), 2.94-2.91 (m, 1H), 2.23 (s, 3H). |
| 56 | 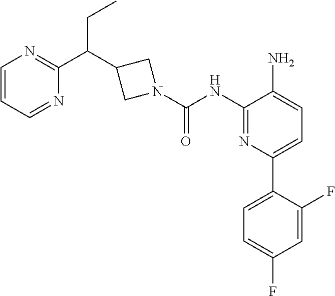 [SMILES: CCC(c1ncccn1)C1CN(C(=O)Nc2nc(-c3ccc(F)cc3F)ccc2N)C1] | 424 | 425 | δ 8.77 (d, J = 5.2 Hz, 2H), 8.43 (s, 1H), 7.93- 7.88 (m, 1H), 7.39- 7.35 (m, 2H), 7.31- 7.25 (m, 1H), 7.18- 7.12 (m, 2H), 5.22 (s, 2H), 4.11 (t, J = 8.4 Hz, 1H), 3.81 (q, J = 5.6 Hz, 2H), 3.56 (t, J = 6.0 Hz, 1H), 3.13- 3.07 (m, 1H), 2.99- 2.93 (m, 1H), 1.73- 1.66 (m, 2H), 0.71 (t, J = 6.8 Hz, 3H). |
| 57 | 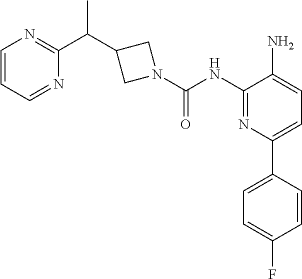 [SMILES: CC(c1ncccn1)C1CN(C(=O)Nc2nc(-c3ccc(F)cc3)ccc2N)C1] | 392 | 393 | δ 8.73 (d, J = 5.2 Hz, 2H), 8.39 (s, 1H), 7.95- 7.91 (m, 2H), 7.49 (d, J = 8.0 Hz, 1H), 7.34 (t, J = 4.8 Hz, 1H), 7.19 (t, J = 8.8 Hz, 2H), 7.11 (d, J = 8.4 Hz, 1H), 5.11 (s, 2H), 4.09 (t, J = 8.4 Hz, 1H), 3.89 (t, J = 8.0 Hz, 1H), 3.81 (q, J = 6.4 Hz, 1H), 3.62 (q, J = 6.4 Hz, 1H), 3.27- 3.18 (m, 1H), 2.49- 2.47 (m, 1H), 1.22 (d, J = 6.8 Hz, 3H). |
| 58 | 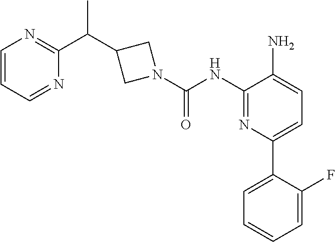 [SMILES: CC(c1ncccn1)C1CN(C(=O)Nc2nc(-c3ccccc3F)ccc2N)C1] | 392 | 393 | δ 8.76 (d, J = 4.8 Hz, 2H), 8.44 (s, 1H), 7.92- 7.87 (m, 1H), 7.41- 7.31 (m, 3H), 7.27- 7.21 (m, 2H), 7.14 (d, J = 8.0 Hz, 1H), 5.23 (s, 2H), 4.11 (t, J = 8.4 Hz, 1H), 3.91 (t, J = 8.0 Hz, 1H), 3.82 (q, J = 6.4 Hz, 1H), 3.65 (q, J = 6.0 Hz, 1H), 3.30- 3.25 (m, 1H), 2.94- 2.92 (m, 1H), 1.24 (d, J = 6.8 Hz, 3H). |
| 59 | 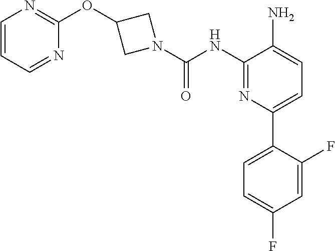 [SMILES: Nc1ccc(-c2ccc(F)cc2F)nc1NC(=O)N1CC(Oc2ncccn2)C1] | 398 | 399 | δ 8.65-8.64 (m, 3H), 7.95-7.89 (m, 1H), 7.38 (dd, J = 8.4, 2.4 Hz, 1H), 7.31-7.25 (m, 1H), 7.22-7.14 (m, 3H), 5.36-5.37 (m, 1H), 5.28 (s, 2H), 4.39 (dd, J = 9.6, 6.8 Hz, 2H), 3.99 (dd, J = 9.6, 3.6 Hz, 2H). |
| 60 | 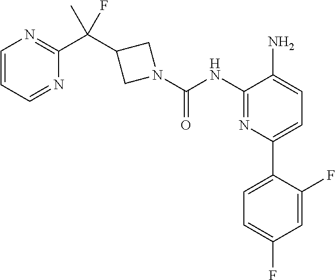 [SMILES: CC(F)(c1ncccn1)C1CN(C(=O)Nc2nc(-c3ccc(F)cc3F)ccc2N)C1] | 428 | 429 | δ 8.89 (d, J = 4.8 Hz, 2H), 8.56 (s, 1H), 7.96- 7.90 (m, 1H), 7.53 (t, J = 4.8 Hz, 1H), 7.37 (dd, J = 6.0, 2.8 Hz, 1H), 7.29 (td, J = 9.2 Hz, J = 2.4 Hz, 1H), 7.18-7.13 (m, 2H), 5.21 (s, 2H), 4.13 (t, J = 9.6 Hz, 1H), 4.03 (t, J = 6.4 Hz, 1H), 3.93 (t, J = 5.6 Hz, 2H), 3.36-29 (m, 1H), 1.67 (d, J = 21.6 Hz, 3H). |
| 61 | 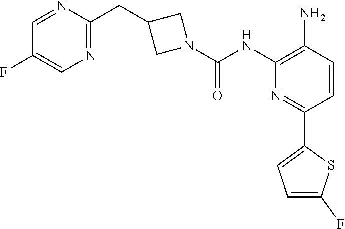 [SMILES: Nc1ccc(-c2ccc(F)s2)nc1NC(=O)N1CC(Cc2ncc(F)cn2)C1] | 402 | 403 | δ 8.83 (s, 2H), 8.44 (s, 1H), 7.46 (d, J = 4.0 Hz, 1H), 7.15 (t, J = 4.0 Hz, 1H), 7.09 (d, J = 4.0 Hz, 1H), 6.66 (q, J = 2.0 Hz, 1H), 5.15 (s, 2H), 4.08 (t, J = 8.0 Hz, 2H), 3.74 (dd, J = 8.0, 6.4 Hz, 2H), 3.25 (d, J = 8.0 Hz, 2H), 3.09-3.01 (m, 1H). |
| 62 | 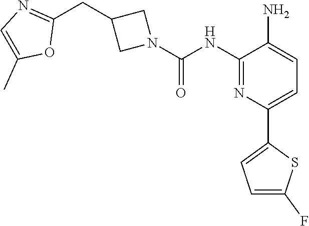 [SMILES: Cc1cnc(CC2CN(C(=O)Nc3nc(-c4ccc(F)s4)ccc3N)C2)o1] | 387 | 388 | δ 8.42 (s, 1H), 7.44 (d, J = 8 Hz, 1H), 7.13 (t, J = 4.0 Hz, 1H), 7.07 (t, J = 8.0 Hz, 1H), 6.69-6.68 (m, 1H), 6.65-6.63 (m, 1H) 5.12 (s, 2H), 4.08- 4.04 (m, 2H), 3.71- 3.67 (m, 2H), 3.00 (d, J = 7.6 Hz, 2H), 2.95- 2.90 (m, 1H), 2.20 (s, 3H). |
| 63 | 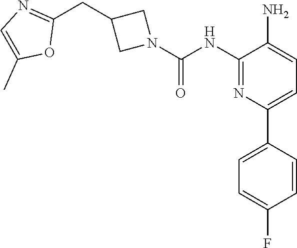 [SMILES: Cc1cnc(CC2CN(C(=O)Nc3nc(-c4ccc(F)cc4)ccc3N)C2)o1] | 381 | 382 | CD₃OD as solvent δ 7.92-7.88 (m, 2H), 7.48 (d, J = 8 Hz, 1H), 7.26 (d, J = 8.4 Hz, 1H), 7.14-7.10 (m, 2H), 6.68 (d, J = 0.8 Hz, 1H) 4.29-4.25 (m, 2H), 3.89-3.86 (m, 2H), 3.10 (s, 3H), 2.30 (s, 3H). |
| 64 | 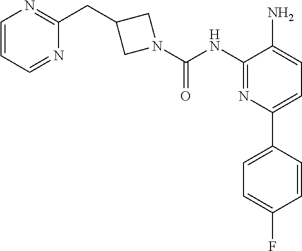 [SMILES: Nc1ccc(-c2ccc(F)cc2)nc1NC(=O)N1CC(Cc2ncccn2)C1] | 378 | 379 | δ 8.75 (d, J = 4.4 Hz, 2H), 8.47 (s, 1H), 7.98- 7.95 (m, 2H), 7.53 (d, J = 8.4 Hz, 1H), 7.37 (t, J = 4.8 Hz, 1H), 7.22 (t, J = 8.8 Hz, 2H), 7.14 (d, J = 8.4 Hz, 1H), 5.16 (s, 2H), 4.11 (t, J = 8.4 Hz, 2H), 3.79-3.75 (m, 2H), 3.24 (d, J = 8.0 Hz, 2H), 3.13- 3.05 (m, 1H). |
| 65 | 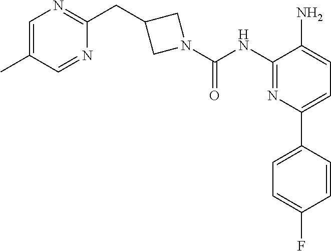 [SMILES: Cc1cnc(CC2CN(C(=O)Nc3nc(-c4ccc(F)cc4)ccc3N)C2)nc1] | 392 | 393 | δ 8.58 (s, 2H), 8.46 (s, 1H), 7.98-7.95 (m, 2H), 7.53 (d, J = 8.4 Hz, 1H), 7.22 (t, J = 8.8 Hz, 2H), 7.14 (d, J = 8.0 Hz, 1H), 5.16 (s, 2H), 4.09 (t, J = 8.4 Hz, 2H), 3.78-3.75 (m, 2H), 3.18 (d, J = 7.6 Hz, 2H), 3.07- 3.03 (m, 1H), 2.24 (s, 3H). |
| 66 | 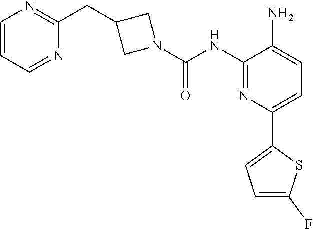 [SMILES: Nc1ccc(-c2ccc(F)s2)nc1NC(=O)N1CC(Cc2ncccn2)C1] | 384 | 385 | δ 8.74 (d, J = 4.8 Hz, 2H), 8.43 (s, 1H), 7.46 (d, J = 4.0 Hz, 1H), 7.37 (t, J = 4.8 Hz, 1H), 7.15 (t, J = 4.0 Hz, 1H), 7.09 (d, J = 4.0 Hz, 1H), 6.66 (q, J = 2.0 Hz, 1H), 5.15 (s, 2H), 4.09 (t, J = 4.0 Hz, 2H), 3.75 (dd, J = 6.4, 1.2 Hz, 2H), 3.23 (d, J = 8.0 Hz, 2H), 3.10-3.05 (m, 1H). |
| 67 | 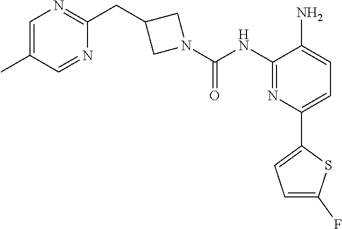 [SMILES: Cc1cnc(CC2CN(C(=O)Nc3nc(-c4ccc(F)s4)ccc3N)C2)nc1] | 398 | 399 | δ 8.58 (d, J = 4.8 Hz, 2H), 8.43 (s, 1H), 7.46 (d, J = 8.0 Hz, 1H), 7.15 (t, J = 4.0 Hz, 1H), 7.09 (d, J = 4.0 Hz, 1H), 6.66 (q, J = 2.0 Hz, 1H), 5.14 (s, 2H), 4.07 (t, J = 8.0 Hz, 2H), 3.75-3.72 (m, 2H), 3.18 (d, J = 4.0 Hz, 2H), 3.07- 3.00 (m, 1H), 2.23 (s, 3H). |
| 68 | 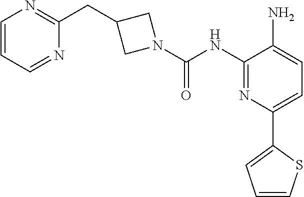 [SMILES: Nc1ccc(-c2cccs2)nc1NC(=O)N1CC(Cc2ncccn2)C1] | 366 | 367 | δ 8.72 (d, J = 5.2 Hz, 2H), 8.41 (s, 1H), 7.45-7.43 (m, 2H), 7.40-7.38 (m, 1H), 7.34 (q, J = 4.8 Hz, 1H), 7.07 (d, J = 8.4 Hz, 1H), 7.03 (dd, J = 4.8, 3.6 Hz, 1H), 5.10 (s, 2H), 4.10-4.06 (m, 2H), 3.75-3.72 (m, 2H), 3.21 (m, J = 6.8 Hz, 2H), 3.07-3.03 (m, 1H). |
| 69 | 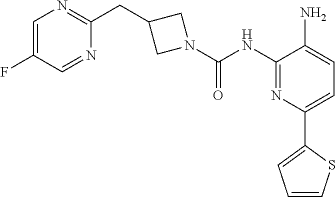 [SMILES: Nc1ccc(-c2cccs2)nc1NC(=O)N1CC(Cc2ncc(F)cn2)C1] | 384 | 385 | δ 8.81 (s, 2H), 8.42 (s, 1H), 7.46-7.44 (m, 2H), 7.39 (d, J = 5.2 Hz, 1H), 7.07 (d, J = 8 Hz, 1H), 7.03 (dd, J = 5.2, 4.0 Hz, 1H), 5.25 (s, 2H), 4.09-4.05 (m, 2H), 3.75-3.71 (m, 2H), 3.23 (d, J = 8 Hz, 2H), 3.05-3.03 (m, 1H). |
| 70 | 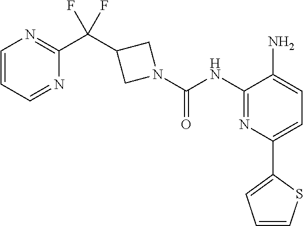 [SMILES: Nc1ccc(-c2cccs2)nc1NC(=O)N1CC(C(F)(F)c2ncccn2)C1] | 402 | 403 | δ 9.01 (s, 2H), 8.66 (s, 1H), 7.73 (d, J = 4.8 Hz, 1H), 7.47 (q, J = 2.4 Hz, 2H), 7.42 (q, J = 4.0 Hz, 1H), 7.12 (d, J = 8.0 Hz, 1H), 7.06 (q, J = 2.4 Hz, 1H), 5.11 (s, 2H), 4.18 (t, J = 8.8 Hz, 2H), 4.05 (t, J = 6.4 Hz, 2H), 3.76-3.67 (m, 1H). |
| 71 | 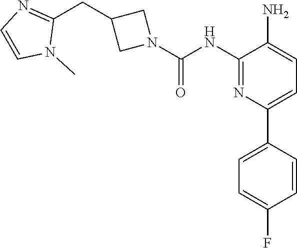 [SMILES: Cn1ccnc1CC1CN(C(=O)Nc2nc(-c3ccc(F)cc3)ccc2N)C1] | 380 | 381 | δ 8.42 (s, 1H), 7.96- 7.93 (m, 2H), 7.50 (d, J = 8.0 Hz, 1H), 7.22- 7.18 (m, 2H), 7.12 (d, J = 8.0 Hz, 1H), 7.00 (s, 1H), 6.71 (s, 1H), 5.14 (s, 2H), 4.07 (t, J = 8.0 Hz, 2H), 3.71-3.68 (m, 2H), 3.54 (s, 3H), 2.97- 2.91 (m, 3H). |
| 72 | 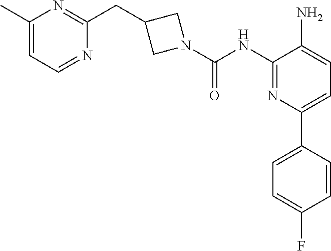 [SMILES: Cc1ccnc(CC2CN(C(=O)Nc3nc(-c4ccc(F)cc4)ccc3N)C2)n1] | 392 | 393 | δ 8.55 (d, J = 4.8 Hz, 1H), 8.42 (s, 1 H), 7.96-7.92 (m, 2H), 7.50 (d, J = 8.0 Hz, 1H), 7.20-7.16 (m, 3H), 7.12 (d, J = 8.0 Hz, 1H), 5.13 (s, 2H), 4.07 (t, J = 8.0 Hz, 2H), 3.76-3.73 (m, 2H), 3.16 (t, J = 8.0 Hz, 2H), 3.07-3.02 (m, 1H), 2.41 (s, 3H). |
| 73 | 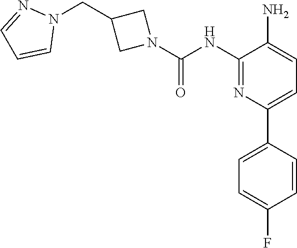 [SMILES: Nc1ccc(-c2ccc(F)cc2)nc1NC(=O)N1CC(Cn2cccn2)C1] | 366 | 367 | δ 8.50 (s, 1H), 7.94- 7.80 (m, 2H), 7.77 (d, J = 1.6 Hz, 1H), 7.53 (d, J = 8 Hz, 1H), 7.45 (d, J = 1.6 Hz, 1H), 7.24-7.19 (m, 2H), 7.15 (d, J = 8 Hz, 1H), 6.24 (t, J = 2 Hz, 1H), 5.14 (s, 2H), 4.37 (d, J = 8.0 Hz, 2H), 4.00 (t, J = 8.0 Hz, 2H), 3.81-3.77 (m, 2H), 3.07- 3.01 (m, 1H). |
| 74 | 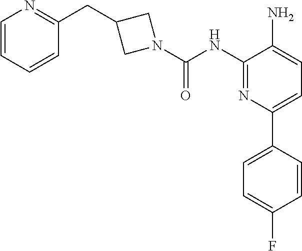 [SMILES: Nc1ccc(-c2ccc(F)cc2)nc1NC(=O)N1CC(Cc2ccccn2)C1] | 377 | 378 | δ 8.47 (d, J = 4.0 Hz, 1H), 8.43 (s, 1H), 7.96-7.93 (m, 2H), 7.72-7.68 (m, 1H), 7.50 (d, J = 8.4 Hz, 1H), 7.26 (d, J = 8.0 Hz, 1H), 7.22-7.18 (m, 3H), 7.13 (d, J = 8.0 Hz, 1H), 5.13 (s, 2H), 4.03 (t, J = 8.0 Hz, 2H), 3.73 (t, J = 5.2 Hz, 2H), 3.06- 2.97 (m, 3H). |
| 75 | 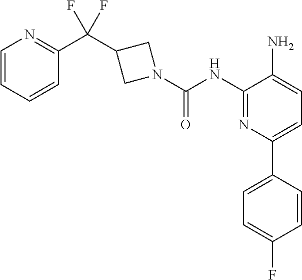 [SMILES: Nc1ccc(-c2ccc(F)cc2)nc1NC(=O)N1CC(C(F)(F)c2ccccn2)C1] | 413 | 414 | δ 8.65 (d, J = 2.2 Hz, 1H), 8.62 (s, 1H), 8.03-7.92 (m, 3H), 7.76 (d, J = 8.0 Hz, 1H), 7.57 (t, J = 4.8 Hz, 1H), 7.52 (d, J = 8.0 Hz, 1H), 7.20 (q, J = 9.2 Hz, 2H), 7.13 (d, J = 8.4 Hz, 1H), 5.12 (s, 2H), 4.12 (t, J = 8.8 Hz, 2H), 4.02 (t, J = 5.6 Hz, 2H), 3.76- 3.66 (m, 1H). |
| 76 | 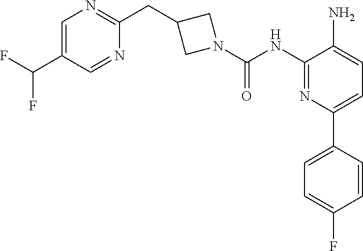 [SMILES: Nc1ccc(-c2ccc(F)cc2)nc1NC(=O)N1CC(Cc2ncc(C(F)F)cn2)C1] | 428 | 429 | δ 8.96 (s, 2H), 8.43 (s, 1H), 7.96-7.92 (m, 2H), 7.50 (d, J = 8.0 Hz, 1H), 7.31-7.03 (m, 4H), 5.13 (s, 2H), 4.10 (t, J = 8.0 Hz, 2H), 3.78-3.76 (m, 2H), 3.31-3.30 (m, 2H), 3.10-3.07 (m, 1H). |
| 77 | 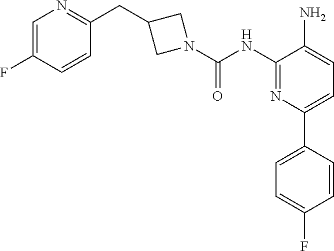 [SMILES: Nc1ccc(-c2ccc(F)cc2)nc1NC(=O)N1CC(Cc2ccc(F)cn2)C1] | 395 | 396 | δ 8.46 (d, J = 2.8 Hz, 1H), 8.43 (s, 1H), 7.96- 7.92 (m, 2H), 7.67- 7.62 (m, 1H), 7.50 (d, J = 8.4 Hz, 1H), 7.37- 7.34 (m, 1H), 7.20 (t, J = 8.8 Hz, 2H), 7.12 (d, J = 8.0 Hz, 1H), 5.12 (s, 2H), 4.03 (t, J = 8.0 Hz, 2H), 3.71 (q, J = 5.6 Hz, 2H), 3.06 (d, J = 7.6 Hz, 2H), 2.98-2.94 (m, 1H). |
| 78 | 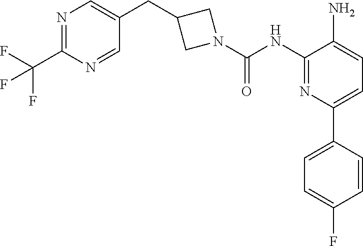 [SMILES: Nc1ccc(-c2ccc(F)cc2)nc1NC(=O)N1CC(Cc2cnc(C(F)(F)F)nc2)C1] | 446 | 447 | δ 8.99 (s, 2H), 8.47 (s, 1H), 7.96-7.93 (m, 2H), 7.50 (d, J = 8.0 Hz, 1H), 7.23-7.18 (m, 2H), 7.13 (d, J = 8.0 Hz, 1H), 5.13 (s, 2H), 4.03 (t, J = 8.0 Hz, 2H), 3.74-3.71 (m, 2H), 3.07 (d, J = 7.6 Hz, 2H), 2.97- 2.93 (m, 1H). |
| 79 | 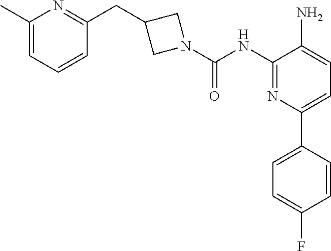 [SMILES: Cc1cccc(CC2CN(C(=O)Nc3nc(-c4ccc(F)cc4)ccc3N)C2)n1] | 391 | 392 | δ 8.42 (s, 1H), 7.94 (q, J = 5.6 Hz, 2H), 7.57 (t, J = 7.6 Hz, 1H), 7.53 (d, J = 8.4 Hz, 1H), 7.20 (t, J = 8.8 Hz, 2H), 7.12 (d, J = 8.4 Hz, 1H), 7.05 (t, J = 8.0 Hz, 2H), 5.13 (s, 2H), 4.02 (t, J = 8.0 Hz, 2H), 3.72 (q, J = 5.2 Hz, 2H), 3.00- 2.92 (m, 3H), 2.41 (s, 3H). |
| 80 | 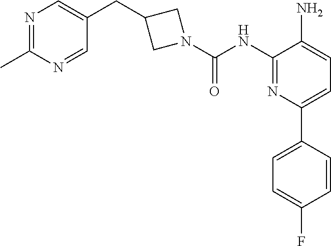 [SMILES: Cc1ncc(CC2CN(C(=O)Nc3nc(-c4ccc(F)cc4)ccc3N)C2)cn1] | 392 | 393 | δ 8.58 (s, 2H), 8.45 (s, 1H), 7.96-7.92 (m, 2H), 7.50 (d, J = 8.0 Hz, 1H), 7.23-7.18 (m, 2H), 7.12 (d, J = 8.0 Hz, 1H), 5.12 (s, 2H), 4.00 (t, J = 8.0 Hz, 2H), 3.69-3.66 (m, 2H), 2.87 (s, 3H), 2.56 (s, 3H). |
| 81 | 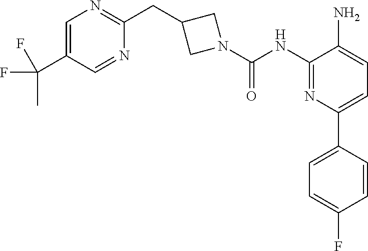 [SMILES: CC(F)(F)c1cnc(CC2CN(C(=O)Nc3nc(-c4ccc(F)cc4)ccc3N)C2)nc1] | 442 | 443 | δ 8.98 (s, 2H), 8.46 (s, 1H), 7.98-7.94 (m, 2H), 7.52 (d, J = 8.0 Hz, 1H), 7.24-7.16 (m, 2H), 7.14 (d, J = 8.0 Hz, 1H), 5.15 (s, 2H), 4.12 (t, J = 8.0 Hz, 2H), 3.80-3.76 (m, 2H), 3.31 (b, 2H); 3.13-3.08 (m, 1H), 2.06 (t, J = 19.2 Hz, 3H). |
| 82 | 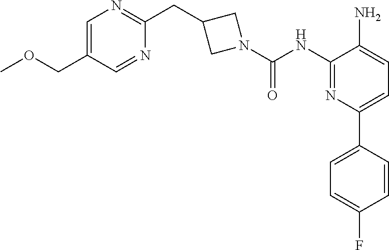 [SMILES: COCc1cnc(CC2CN(C(=O)Nc3nc(-c4ccc(F)cc4)ccc3N)C2)nc1] | 422 | 423 | δ 8.68 (s, 2H), 8.43 (s, 1H), 7.96-7.92 (m, 2H), 7.50 (d, J = 8.0 Hz, 1H), 7.22 (t, J = 2.0 Hz, 2H), 7.12 (d, J = 8.0 Hz, 1H), 5.13 (s, 2H), 4.42 (s, 2H), 4.08 (t, J = 8.0 Hz, 2H), 3.74 (q, J = 6.4 Hz, 2H), 3.26 (s, 3H), 3.22 (d, J = 8.0 Hz, 2H), 3.09-3.04 (m, 1H). |
| 83 | 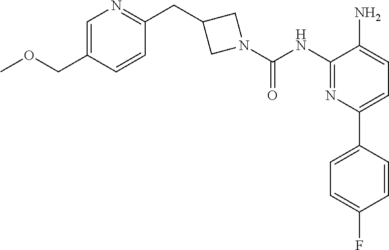 [SMILES: COCc1ccc(CC2CN(C(=O)Nc3nc(-c4ccc(F)cc4)ccc3N)C2)nc1] | 421 | 422 | δ 8.42 (d, J = 4 Hz, 2H), 7.96-7.92 (m, 2H), 7.65-7.63 (m, 1H), 7.50 (d, J = 8.4 Hz, 1H), 7.26-7.18 (m, 3H), 7.12 (d, J = 8 Hz, 1H), 5.12 (s, 2H), 4.38 (s, 2H); 4.03 (t, J = 8 Hz, 2H), 3.74- 3.71 (m, 2H), 3.26 (s, 3H), 3.05-3.04 (m, 2H), 3.00-2.95 (m, 1H). |
| 84 | 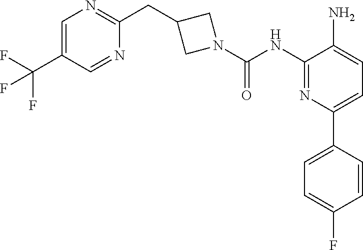 [SMILES: Nc1ccc(-c2ccc(F)cc2)nc1NC(=O)N1CC(Cc2ncc(C(F)(F)F)cn2)C1] | 446 | 447 | δ 9.22 (s, 2H), 8.47 (s, 1H), 7.98-7.95 (m, 2H), 7.53 (d, J = 8.4 Hz, 1H), 7.22 (t, J = 8.8 Hz, 2H), 7.14 (d, J = 8.4 Hz, 1H), 5.16 (s, 2H), 4.14 (t, J = 8 Hz, 2H), 3.81-3.77 (m, 2H), 3.38 (d, J = 7.6 Hz, 2H), 3.14-3.10 (m, 1H). |
| 85 | 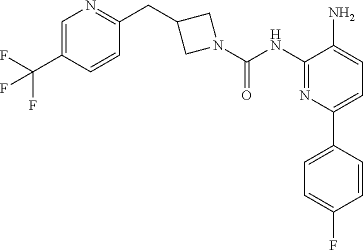 [SMILES: Nc1ccc(-c2ccc(F)cc2)nc1NC(=O)N1CC(Cc2ccc(C(F)(F)F)cn2)C1] | 445 | 446 | δ 8.87 (s, 1H), 8.44 (s, 1H), 8.14-8.11 (m, 1H), 7.96-7.92 (m, 2H), 7.55-7.50 (m, 2H), 7.22 (t, J = 8.8 Hz, 2H), 7.14, 7.12 (dd, J = 8.4 Hz, 4.0 Hz, 1H), 5.13 (s, 2H), 4.05 (t, J = 8 Hz, 2H), 3.76-3.72 (m, 2H), 3.20 (d, J = 8.0 Hz, 2H), 3.05-3.01 (m, 1H). |
| 86 | 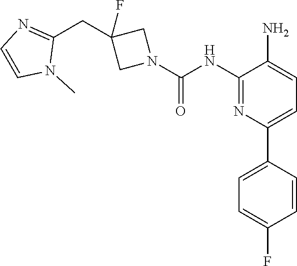 [SMILES: Cn1ccnc1CC1(F)CN(C(=O)Nc2nc(-c3ccc(F)cc3)ccc2N)C1] | 398 | 399 | δ 8.67 (s, 1H), 7.96- 7.92 (m, 2 H), 7.52 (d, J = 8.0 Hz, 1H), 7.20 (t, J = 8.4 Hz, 2H), 7.13 (d, J = 8.0 Hz, 1H), 7.05 (s, 1H), 6.78 (s, 1H), 5.16 (s, 2H), 4.31 (q, J = 10.8 Hz, 2H), 4.03 (q, J = 10.8 Hz, 2H), 3.56 (s, 3H), 3.33 (d, J = 20.8 Hz, 2H). |
| 87 | 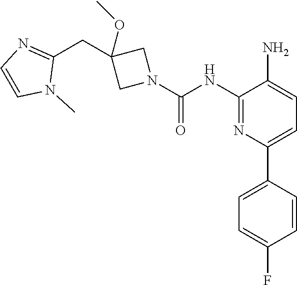 [SMILES: COC1(Cc2nccn2C)CN(C(=O)Nc2nc(-c3ccc(F)cc3)ccc2N)C1] | 410 | 411 | δ 8.56 (s, 1H), 7.96- 7.93 (m, 2 H), 7.51 (d, J = 8.4 Hz, 1H), 7.20 (t, J = 8.8 Hz, 2H), 7.13 (d, J = 8.0 Hz, 1H), 7.01 (s, 1H), 6.76 (s, 1H), 5.13 (s, 2H), 4.08 (d, J = 9.2 Hz, 2H), 3.95 (d, J = 9.6 Hz, 2H), 3.57 (s, 3H), 3.24 (s, 3H), 3.12 (s, 2H). |

### HDAC2 and HDAC1 Enzymatic Assay (HDAC2 and HDAC1 IC50 Data)

The following describes an assay protocol for measuring the deacetylation of a peptide substrate by HDAC2 or HDAC1.

HD AC protein composition and respective substrate peptides are summarized below.

| Assay | | Regulatory | Substrate |
|---|---|---|---|
| name | Expression Construct | subunit | peptide |
| HDAC1 | Full length Human HDAC1 | None | FAM- |
| | with C-terminal His-tag and | | TSRHK(Ac)KL- |
| | C-terminal FLAG-tag, | | NH2 |
| | expressed in baculovirus | | |
| | expression system. | | |
| HDAC2 | Full length Human HDAC2 | None | FAM- |
| | with C-terminal FLAG-tag, | | TSRHK(Ac)KL- |
| | expressed in baculovirus | | NH2 |
| | expression system. | | |

Assay Set Up:

HD AC reactions are assembled in 384 well plates (Greiner) in a total volume of 20 μL as following:

HDAC proteins are pre-diluted in the assay buffer comprising: 100 mM HEPES, pH 7.5, 0.1% BSA, 0.01% Triton X-100, 25 mM KCl and dispensed into 384 well plate (10 uL per well).

Test compounds are serially pre-diluted in DMSO and added to the protein samples by acoustic dispensing (Labcyte Echo). Concentration of DMSO is equalized to 1% in all samples.

Control samples (0%-inhibition in the absence of inhibitor, DMSO only) and 100%-inhibition (in the absence of enzyme) are assembled in replicates of four and used to calculate the %-inhibition in the presence of compounds.

At this step compounds can be pre-incubated with enzyme if desired.

The reactions are initiated by addition of 10 uL of the FAM-labeled substrate peptide pre-diluted in the same assay buffer. Final concentration of substrate peptide is 1 uM (HDAC1-2).

The reactions are allowed to proceed at room temperature. Following incubation, the reactions are quenched by addition of 50 μL of termination buffer (100 mM HEPES, pH7.5, 0.01% Triton X-100, 0.1% SDS). Terminated plates are analyzed on a microfluidic electrophoresis instrument (Caliper LabChip® 3000, Caliper Life Sciences/Perkin Elmer) which enables electrophoretic separation of de-acetylated product from acetylated substrate. A change in the relative intensity of the peptide substrate and product is the parameter measured. Activity in each test sample is determined as the product to sum ratio (PSR): P/(S+P), where P is the peak height of the product, and S is the peak height of the substrate. Percent inhibition (Pinh) is determined using the following equation: Pinh=(PSR0% inh−PSRcompound)/(PSR0% inh−PSR100% inh)*100, in which: PSRcompound is the product/sum ratio in the presence of compound, PSR0% inh is the product/sum ratio in the absence of compound and the PSR100% inh is the product/sum ratio in the absence of the enzyme. To determine IC50 of compounds (50%-inhibition) the %-inh data (Pinh versus compound concentration) are fit by a 4 parameter sigmoid dose-response model using XLfit software (IDBS).

The results of this assay for certain compounds are reported in Table 2, below. In the table, “A” indicates a IC50 value of less than 0.5 μM; “B” a IC50 value from 0.5 μM to 1.0 μM; “C” a IC50 value of greater than 1.0 μM and less than or equal to 2.0 μM; and “D” indicates an IC50 value of greater than 2.0 μM. NT=Not Tested.

TABLE 2

| | HDAC2 | HDAC1 |
|---|---|---|
| Compound | IC50, | IC50, |
| No. | (uM) | (uM) |
| 1 | B | A |
| 2 | B | A |
| 3 | C | B |
| 4 | D | D |
| 5 | C | C |
| 6 | D | D |
| 7 | D | D |
| 8 | NT | NT |
| 9 | D | C |
| 10 | NT | NT |
| 11 | B | B |
| 12 | B | B |
| 13 | C | C |
| 14 | B | A |
| 15 | D | D |
| 16 | C | B |
| 17 | NT | NT |
| 18 | NT | NT |
| 19 | C | C |
| 20 | NT | NT |
| 21 | B | A |
| 22 | B | B |
| 23 | B | C |
| 24 | C | B |
| 25 | NT | NT |
| 26 | B | B |
| 27 | A | A |
| 28 | NT | NT |
| 29 | NT | NT |
| 30 | C | C |
| 31 | D | D |
| 32 | NT | NT |
| 33 | B | B |
| 34 | B | B |
| 35 | B | C |
| 36 | A | A |
| 37 | B | B |
| 38 | A | A |
| 39 | NT | NT |
| 40 | C | B |
| 41 | NT | NT |
| 42 | C | B |
| 43 | D | B |
| 44 | D | D |
| 45 | C | B |
| 46 | C | C |
| 47 | C | B |
| 48 | NT | NT |
| 49 | NT | NT |
| 50 | NT | NT |
| 51 | NT | NT |
| 52 | NT | NT |
| 53 | NT | NT |
| 54 | NT | NT |
| 55 | NT | NT |
| 56 | NT | NT |
| 57 | B | A |
| 58 | NT | NT |
| 59 | B | A |
| 60 | NT | NT |
| 61 | B | A |
| 62 | NT | NT |
| 63 | NT | NT |
| 64 | C | A |
| 65 | B | A |
| 66 | B | A |
| 67 | NT | NT |
| 68 | C | A |
| 69 | C | A |
| 70 | B | A |
| 71 | D | B |
| 72 | B | A |
| 73 | C | B |
| 74 | B | A |
| 75 | NT | NT |
| 76 | B | A |
| 77 | C | A |
| 78 | D | D |
| 79 | C | B |
| 80 | C | B |
| 81 | B | A |
| 82 | B | A |
| 83 | B | A |
| 84 | 1.19 | 0.346 |
| 85 | NT | NT |
| 86 | D | C |
| 87 | D | D |

### HDAC2 Enzymatic Inhibition Assay in SH-SY5Y Cell Lysate with an Exogenous Substrate

SH-SY5Y cells (Sigma) were cultured in Eagle's Modified Essential Medium supplemented with 10% fetal bovine serum and pen/strep. Twenty-four hours prior to compound dosing 20 uL of cells were plated in white 384 well plates at a density of 1,500 cells/well. Compounds were serially diluted in neat DMSO and then diluted 1:100 v/v into media without FBS and mixed. Media was removed from the plated cells and the diluted compounds in serum free media (1% v/v final DMSO) were added and incubated at 37° C. for five hours. Ten uL of HDAC-Glo 2 reagent with 0.1% Triton X-100 was then added, the plate was mixed and allowed to develop at room temperature for 100 minutes. Plates were then read with a Spectramax LMax luminometer employing a 0.4 s integration time. Dose response curves were constructed with normalized data where CI-994 at 100 uM was defined as 100% inhibition and DMSO alone as 0% inhibition.

The results of this assay for certain compounds are reported in Table 3, below. In the table, “A” indicates a IC50 value of less than 0.5 μM; “B” a IC50 value from 0.5 μM to 1.0 μM; “C” a IC50 value of greater than 1.0 μM and less than or equal to 2.0 μM; and “D” indicates an IC50 value of greater than 2.0 μM. NT=Not Tested.

TABLE 3

| | HDAC2 IC50, |
|---|---|
| Compound | SH-SY5Y Cell |
| No. | Lysate (uM) |
| 1 | A |
| 2 | C |
| 3 | C |
| 4 | C |
| 5 | D |
| 6 | C |
| 7 | C |
| 8 | D |
| 9 | D |
| 10 | D |
| 11 | B |
| 12 | C |
| 13 | C |
| 14 | B |
| 15 | D |
| 16 | B |
| 17 | D |
| 18 | D |
| 19 | B |
| 20 | D |
| 21 | B |
| 22 | B |
| 23 | B |
| 24 | A |
| 25 | D |
| 26 | C |
| 27 | B |
| 28 | D |
| 29 | D |
| 30 | C |
| 31 | D |
| 32 | C |
| 33 | C |
| 34 | D |
| 35 | B |
| 36 | C |
| 37 | B |
| 38 | B |
| 39 | D |
| 40 | B |
| 41 | C |
| 42 | A |
| 43 | D |
| 44 | D |
| 45 | B |
| 46 | D |
| 47 | C |
| 48 | D |
| 49 | D |
| 50 | D |
| 51 | D |
| 52 | D |
| 53 | D |
| 54 | C |
| 55 | D |
| 56 | D |
| 57 | C |
| 58 | C |
| 59 | D |
| 60 | D |
| 61 | C |
| 62 | D |
| 63 | C |
| 64 | B |
| 65 | B |
| 66 | C |
| 67 | B |
| 68 | B |
| 69 | C |
| 70 | C |
| 71 | B |
| 72 | C |
| 73 | C |
| 74 | C |
| 75 | D |
| 76 | C |
| 77 | D |
| 78 | D |
| 79 | D |
| 80 | C |
| 81 | D |
| 82 | C |
| 83 | C |
| 84 | D |
| 85 | D |
| 86 | C |
| 87 | C |

### Comparison of Methylene-Linked Heteroaromatic Rings to Directly Linked Heteroaromatic 3-Substituted Azetidineureas

Table 4 below shows a comparison of the activity levels between certain inventive compounds and those failing to possess the spacer group between the azetidinyl motif and R¹ (i.e., variable “X” in the compounds of Formula I). As shown by the data, there is a decrease in potency in the HDAC2 SH-SY5Y cell lysate assay as well as in the HDAC2 and HDAC1 recombinant enzymatic activity assays when the compounds lack the methylene group for variable X. For example, Compound 1 is 100-fold more potent in the SH-SY5Y cell assay, >7-fold more potent in the HDAC2 recombinant enzymatic assay, and 10-fold more potent in the HDAC1 recombinant enzymatic assay in comparison to the corresponding compound Comparator A, which has the pyrimidine ring directly linked at the 3-position of the azetidine. A similar trend is seen for other matched pairs in Table 4. Compound 6 with a methylene linker is >10-fold more potent in all assays than Comparator B. Compound 14 with a methylene linker is >10-fold more potent in all assays than Comparator C.

TABLE 4

| | | HDAC2 | HDAC2 | HDAC1 |
|---|---|---|---|---|
| | | IC50, SH-SY5Y Cell | IC50, | IC50, |
| No. | Structure | Lysate (uM) | (uM) | (uM) |
| 1 | 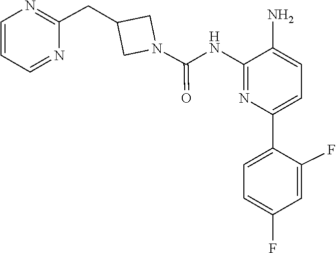 [SMILES: Nc1ccc(-c2ccc(F)cc2F)nc1NC(=O)N1CC(Cc2ncccn2)C1] | 0.496 | 0.70 | 0.454 |
| Comparator A | 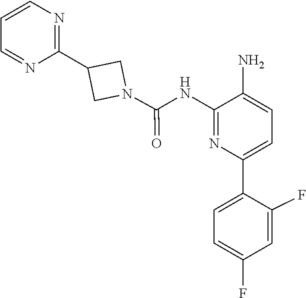 [SMILES: Nc1ccc(-c2ccc(F)cc2F)nc1NC(=O)N1CC(c2ncccn2)C1] | >52 | 5.29 | 4.58 |
| 6 | 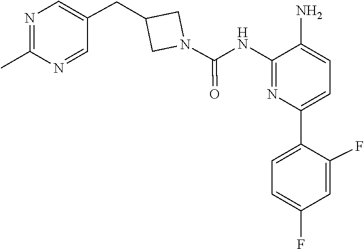 [SMILES: Cc1ncc(CC2CN(C(=O)Nc3nc(-c4ccc(F)cc4F)ccc3N)C2)cn1] | 1.56 | 2.8 | 2.26 |
| Comparator B | 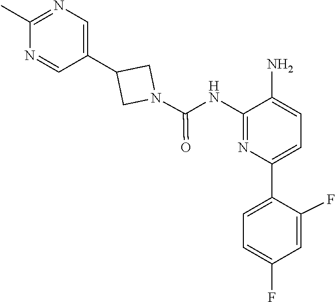 [SMILES: Cc1ncc(C2CN(C(=O)Nc3nc(-c4ccc(F)cc4F)ccc3N)C2)cn1] | 30 | >30 | >30 |
| 14 | 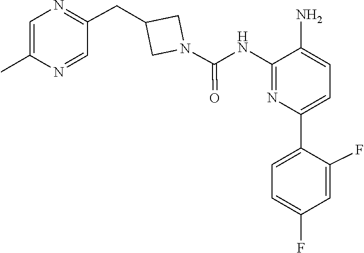 [SMILES: Cc1cnc(CC2CN(C(=O)Nc3nc(-c4ccc(F)cc4F)ccc3N)C2)cn1] | 0.687 | 0.583 | 0.406 |
| Comparator C | 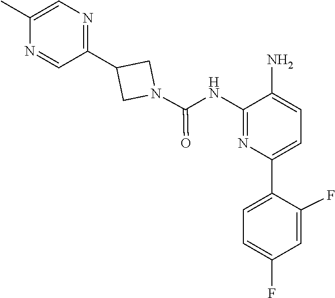 [SMILES: Cc1cnc(C2CN(C(=O)Nc3nc(-c4ccc(F)cc4F)ccc3N)C2)cn1] | 9.7 | 13.5 | 13.9 |

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 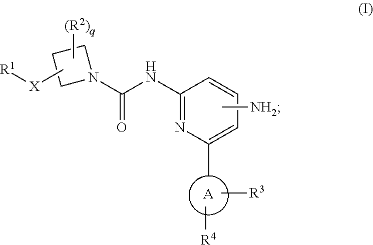
or a pharmaceutically acceptable salt thereof, wherein
ring A is phenyl or thiopheneyl;
X is (CR^{a}R^{b})ₜ, O, or NR⁵;
q is 0, 1, or 2;
t is 1, 2, or 3;
R¹ is phenyl or heteroaryl, each of which are optionally substituted with 1 to 3 groups selected from R^{c};
R² is halo, (C₁-C₄)alkyl, (C₁-C₄)alkoxy, or OH;
R³ is hydrogen or halo;
R⁴ is halo when ring A is phenyl and R⁴ is hydrogen when ring A is thiopheneyl;
R⁵ is hydrogen, (C₁-C₄)alkyl, or (C₁-C₄)alkylO(C₁-C₄)alkyl;
R^{a} and R^{b} are each independently hydrogen, (C₁-C₄)alkyl, halo(C₁-C₄)alkyl, (C₁-C₄)alkoxy, or halo; and
R^{c} is halo, (C₁-C₄)alkyl, halo(C₁-C₄)alkyl, (C₁-C₄)alkoxy, halo(C₁-C₄)alkoxy, (C₁-C₄)alkylO(C₁-C₄)alkyl, (C₁-C₄)alkylNH(C₁-C₄)alkyl, (C₁-C₄)alkylN((C₁-C₄)alkyl)₂, —(C₁-C₄)alkylheteroaryl, or —(C₁-C₄)alkylheterocyclyl, wherein said heteroaryl and heterocyclyl are each optionally and independently substituted with 1 to 3 groups selected from (C₁-C₄)alkyl, halo(C₁-C₄)alkyl, (C₁-C₄)alkoxy, and halo.

2. The compound of claim 1, wherein the compound is of the Formula II or IIa: 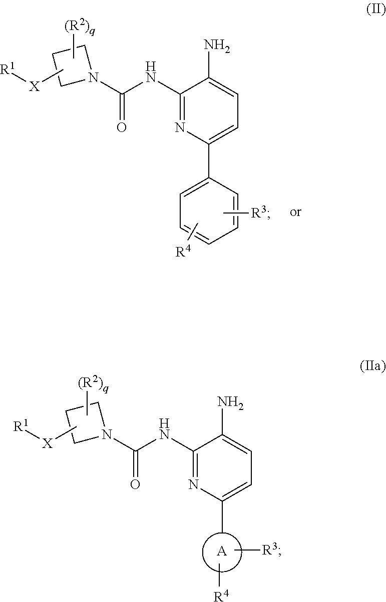
or a pharmaceutically acceptable salt thereof.

3. The compound of claim 1, wherein the compound is of the Formula III or IIIa: 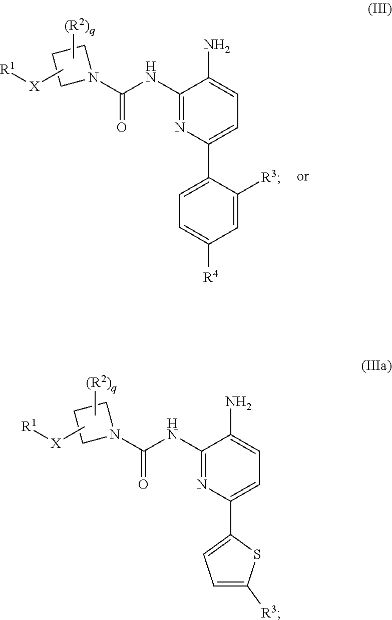
or a pharmaceutically acceptable salt thereof.

4. The compound of claim 1, wherein the compound is of the Formula IV or IVa: 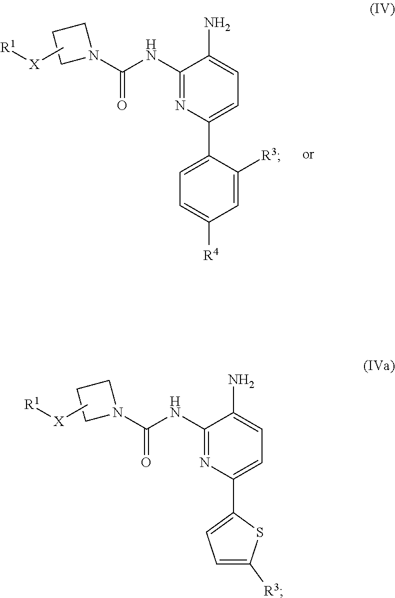
or a pharmaceutically acceptable salt thereof.

5. The compound of claim 1, wherein R³ is halo.

6. The compound of claim 1, wherein R³ is fluoro.

7. The compound of claim 1, wherein R³ is hydrogen.

8. The compound of claim 1, wherein R⁴ is fluoro.

9. The compound of claim 1, wherein X is (CR^{a}R^{b})ₜ.

10. The compound of claim 1, wherein R^{a} is hydrogen, (C₁-C₄)alkyl, or halo; and R^{b} is hydrogen or halo.

11. The compound of claim 1, wherein R^{a} is hydrogen, methyl, or fluoro; and R^{b} is hydrogen or fluoro.

12. The compound of claim 1, wherein R^{a} is hydrogen and R^{b} is halo.

13. The compound of claim 12, wherein R^{b} is fluoro.

14. The compound of claim 1, wherein R^{a} is halo and R^{b} is halo.

15. The compound of claim 14, wherein R^{a} and R^{b} are each fluoro.

16. The compound of claim 1, wherein t is 1 or 2.

17. The compound of claim 1, wherein the compound of the Formula V or Va: 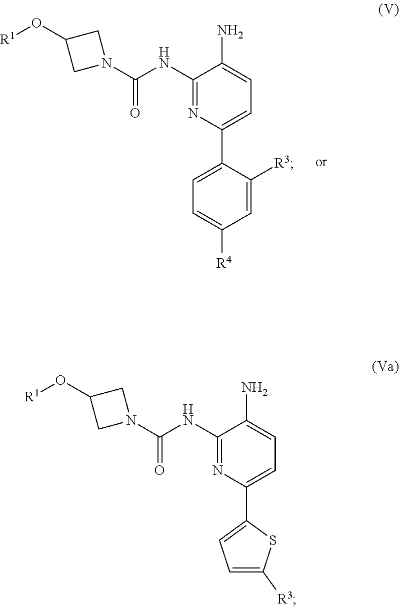
or a pharmaceutically acceptable salt thereof.

18. The compound of claim 1, wherein the compound of the Formula VI or VIa: 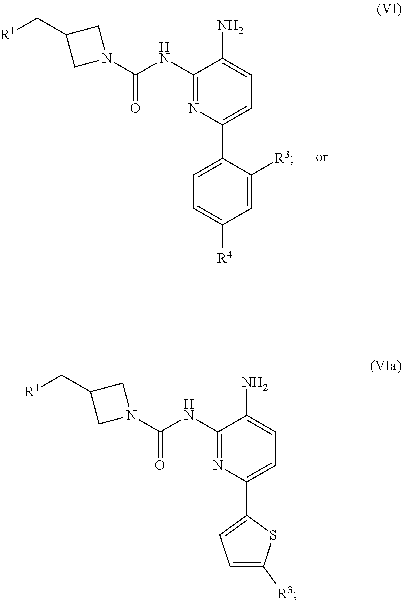
or a pharmaceutically acceptable salt thereof.

19. The compound of claim 1, wherein R¹ is heteroaryl optionally substituted with 1 to 2 groups selected from R^{c}.

20. The compound of claim 1, wherein R¹ is pyrimidinyl, pyridinyl, imidazopyridinyl, pyrazinyl, pyrazolyl, imidazolyl, oxazolyl, thiazolyl, or thiadiazolyl, each of which is optionally substituted with 1 to 2 groups selected from R^{c}.

21. The compound of claim 1, wherein R^{c} is halo, halo(C₁-C₄)alkyl, (C₁-C₄)alkyl, or (C₁-C₄)alkylO(C₁-C₄)alkyl.

22. The compound of claim 1, wherein R^{c} is fluoro, CF₃, methyl, or CH₂OCH₃.

23. The compound of claim 1, wherein the compound is selected from 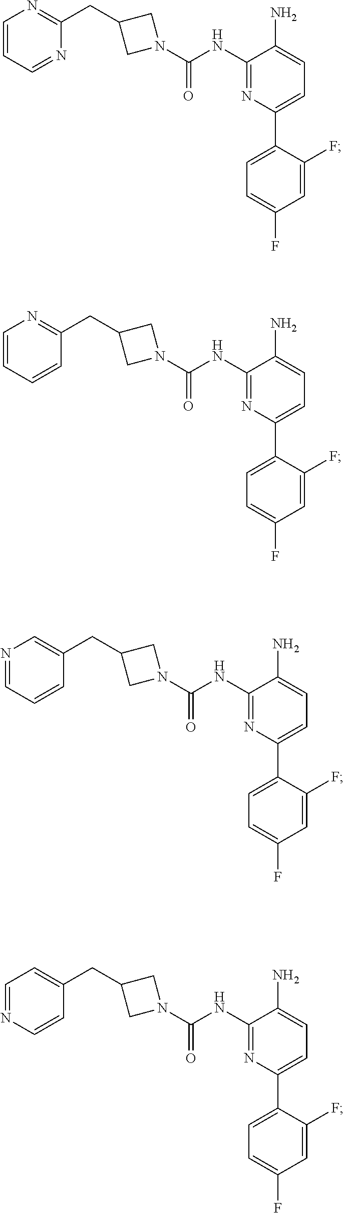 [SMILES: Nc1ccc(-c2ccc(F)cc2F)nc1NC(=O)N1CC(Cc2ncccn2)C1 ; Nc1ccc(-c2ccc(F)cc2F)nc1NC(=O)N1CC(Cc2ccccn2)C1 ; Nc1ccc(-c2ccc(F)cc2F)nc1NC(=O)N1CC(Cc2cccnc2)C1 ; Nc1ccc(-c2ccc(F)cc2F)nc1NC(=O)N1CC(Cc2ccncc2)C1] 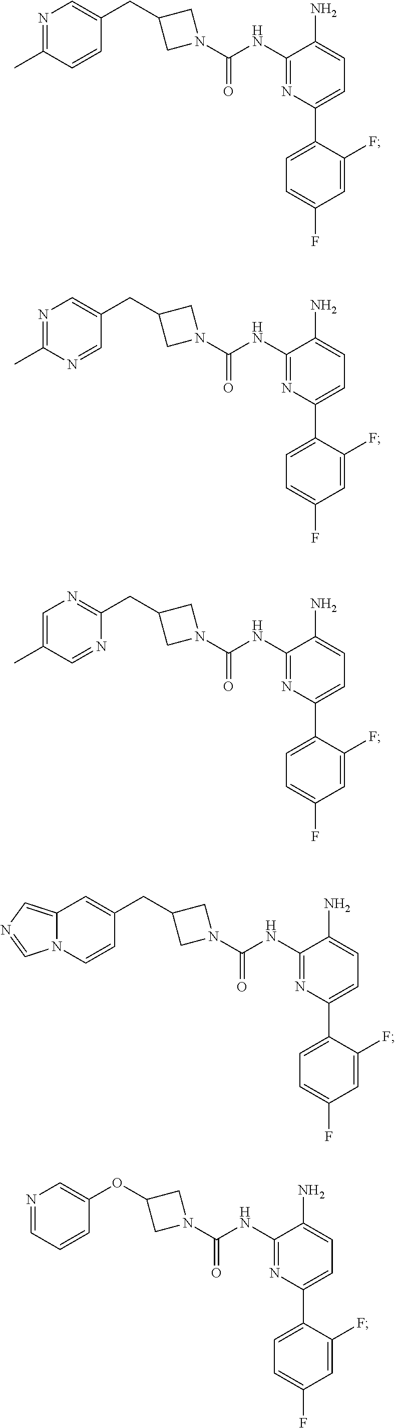 [SMILES: Cc1ccc(CC2CN(C(=O)Nc3nc(-c4ccc(F)cc4F)ccc3N)C2)cn1 ; Cc1ncc(CC2CN(C(=O)Nc3nc(-c4ccc(F)cc4F)ccc3N)C2)cn1 ; Cc1cnc(CC2CN(C(=O)Nc3nc(-c4ccc(F)cc4F)ccc3N)C2)nc1 ; Nc1ccc(-c2ccc(F)cc2F)nc1NC(=O)N1CC(Cc2ccn3cncc3c2)C1 ; Nc1ccc(-c2ccc(F)cc2F)nc1NC(=O)N1CC(Oc2cccnc2)C1] 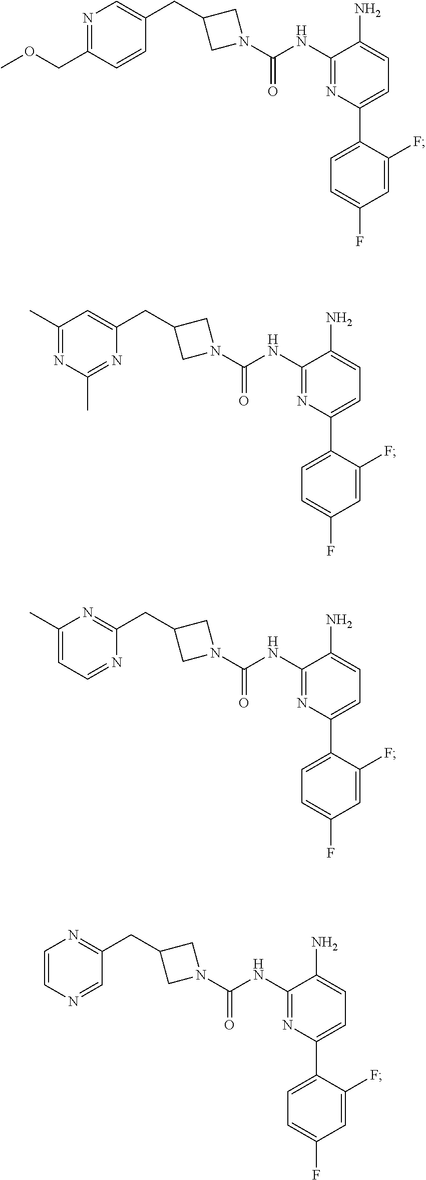 [SMILES: COCc1ccc(CC2CN(C(=O)Nc3nc(-c4ccc(F)cc4F)ccc3N)C2)cn1 ; Cc1cc(CC2CN(C(=O)Nc3nc(-c4ccc(F)cc4F)ccc3N)C2)nc(C)n1 ; Cc1ccnc(CC2CN(C(=O)Nc3nc(-c4ccc(F)cc4F)ccc3N)C2)n1 ; Nc1ccc(-c2ccc(F)cc2F)nc1NC(=O)N1CC(Cc2cnccn2)C1] 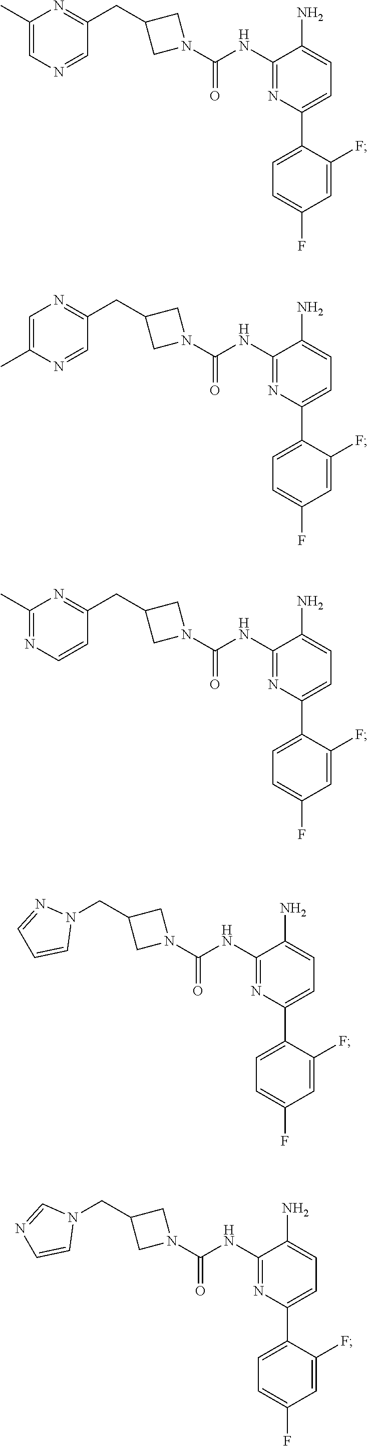 [SMILES: Cc1cncc(CC2CN(C(=O)Nc3nc(-c4ccc(F)cc4F)ccc3N)C2)n1 ; Cc1cnc(CC2CN(C(=O)Nc3nc(-c4ccc(F)cc4F)ccc3N)C2)cn1 ; Cc1nccc(CC2CN(C(=O)Nc3nc(-c4ccc(F)cc4F)ccc3N)C2)n1 ; Nc1ccc(-c2ccc(F)cc2F)nc1NC(=O)N1CC(Cn2cccn2)C1 ; Nc1ccc(-c2ccc(F)cc2F)nc1NC(=O)N1CC(Cn2ccnc2)C1] 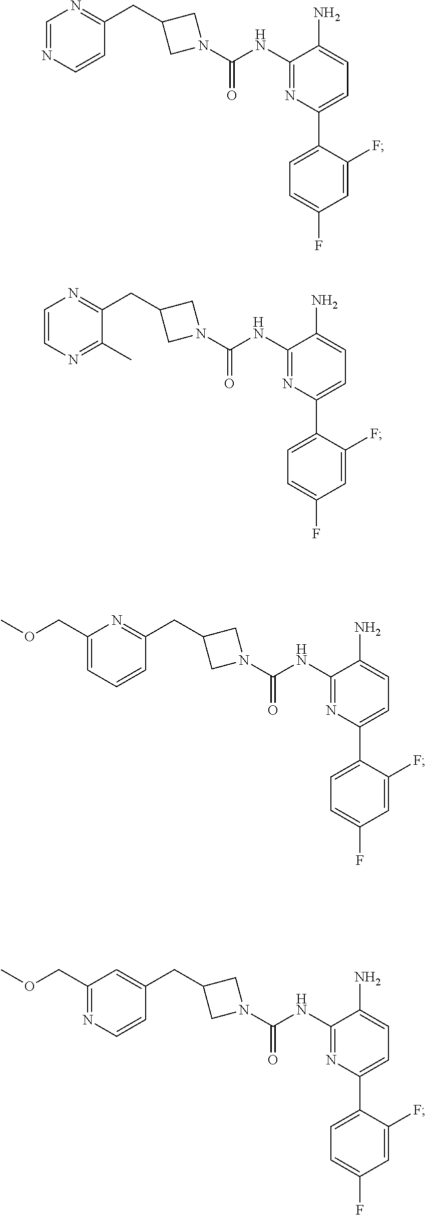 [SMILES: Nc1ccc(-c2ccc(F)cc2F)nc1NC(=O)N1CC(Cc2ccncn2)C1 ; Cc1nccnc1CC1CN(C(=O)Nc2nc(-c3ccc(F)cc3F)ccc2N)C1 ; COCc1cccc(CC2CN(C(=O)Nc3nc(-c4ccc(F)cc4F)ccc3N)C2)n1 ; COCc1cc(CC2CN(C(=O)Nc3nc(-c4ccc(F)cc4F)ccc3N)C2)ccn1] 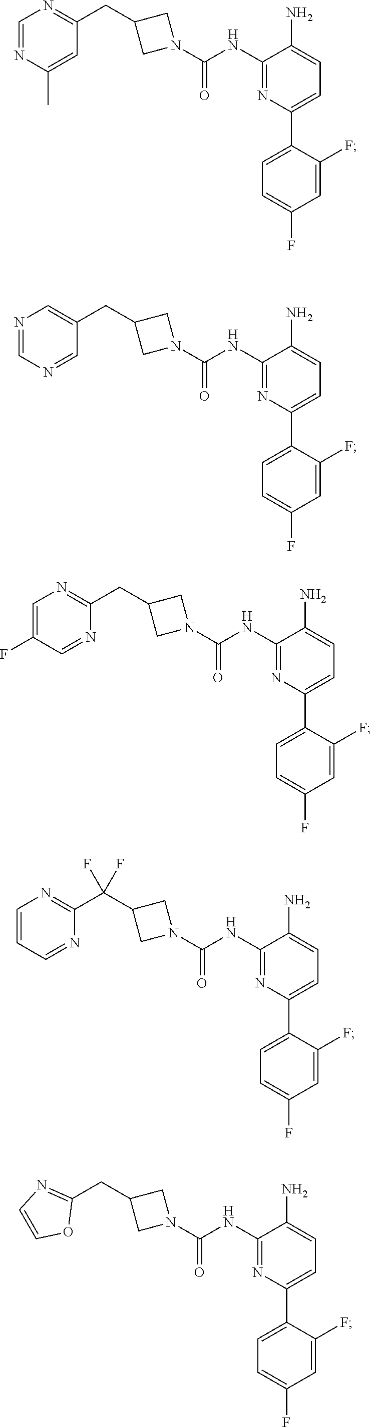 [SMILES: Cc1cc(CC2CN(C(=O)Nc3nc(-c4ccc(F)cc4F)ccc3N)C2)ncn1 ; Nc1ccc(-c2ccc(F)cc2F)nc1NC(=O)N1CC(Cc2cncnc2)C1 ; Nc1ccc(-c2ccc(F)cc2F)nc1NC(=O)N1CC(Cc2ncc(F)cn2)C1 ; Nc1ccc(-c2ccc(F)cc2F)nc1NC(=O)N1CC(C(F)(F)c2ncccn2)C1 ; Nc1ccc(-c2ccc(F)cc2F)nc1NC(=O)N1CC(Cc2ncco2)C1] 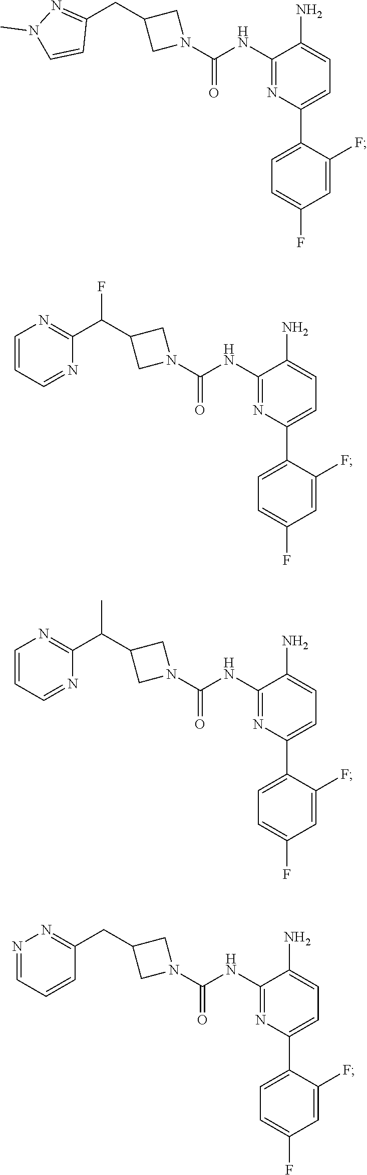 [SMILES: Cn1ccc(CC2CN(C(=O)Nc3nc(-c4ccc(F)cc4F)ccc3N)C2)n1 ; Nc1ccc(-c2ccc(F)cc2F)nc1NC(=O)N1CC(C(F)c2ncccn2)C1 ; CC(c1ncccn1)C1CN(C(=O)Nc2nc(-c3ccc(F)cc3F)ccc2N)C1 ; Nc1ccc(-c2ccc(F)cc2F)nc1NC(=O)N1CC(Cc2cccnn2)C1] 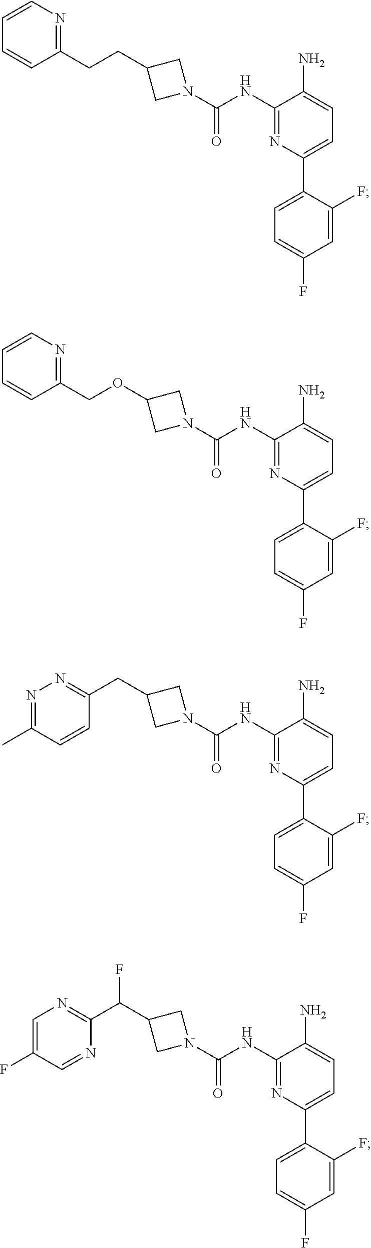 [SMILES: Nc1ccc(-c2ccc(F)cc2F)nc1NC(=O)N1CC(CCc2ccccn2)C1 ; Nc1ccc(-c2ccc(F)cc2F)nc1NC(=O)N1CC(OCc2ccccn2)C1 ; Cc1ccc(CC2CN(C(=O)Nc3nc(-c4ccc(F)cc4F)ccc3N)C2)nn1 ; Nc1ccc(-c2ccc(F)cc2F)nc1NC(=O)N1CC(C(F)c2ncc(F)cn2)C1] 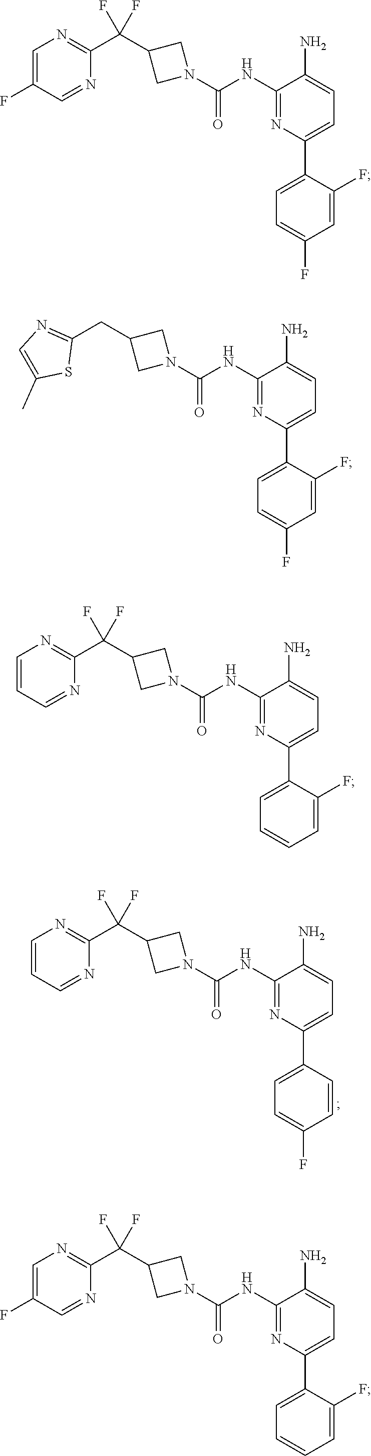 [SMILES: Nc1ccc(-c2ccc(F)cc2F)nc1NC(=O)N1CC(C(F)(F)c2ncc(F)cn2)C1 ; Cc1cnc(CC2CN(C(=O)Nc3nc(-c4ccc(F)cc4F)ccc3N)C2)s1 ; Nc1ccc(-c2ccccc2F)nc1NC(=O)N1CC(C(F)(F)c2ncccn2)C1 ; Nc1ccc(-c2ccc(F)cc2)nc1NC(=O)N1CC(C(F)(F)c2ncccn2)C1 ; Nc1ccc(-c2ccccc2F)nc1NC(=O)N1CC(C(F)(F)c2ncc(F)cn2)C1] 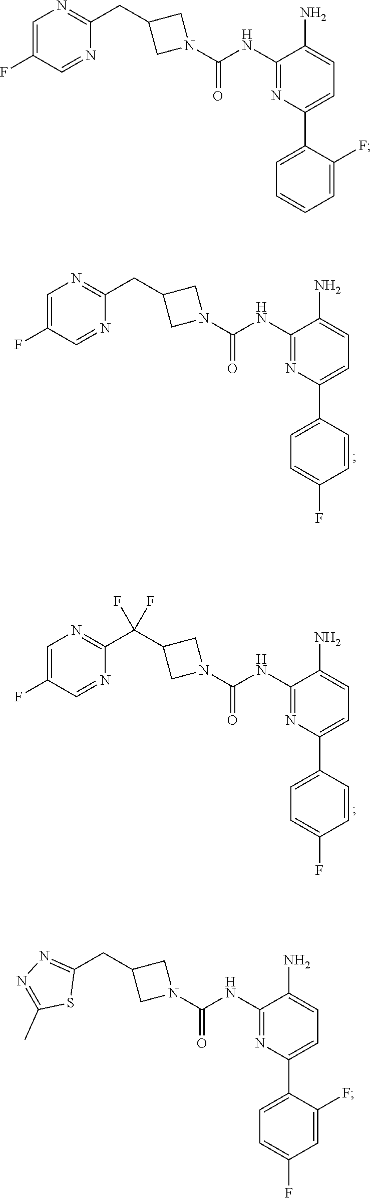 [SMILES: Nc1ccc(-c2ccccc2F)nc1NC(=O)N1CC(Cc2ncc(F)cn2)C1 ; Nc1ccc(-c2ccc(F)cc2)nc1NC(=O)N1CC(Cc2ncc(F)cn2)C1 ; Nc1ccc(-c2ccc(F)cc2)nc1NC(=O)N1CC(C(F)(F)c2ncc(F)cn2)C1 ; Cc1nnc(CC2CN(C(=O)Nc3nc(-c4ccc(F)cc4F)ccc3N)C2)s1] 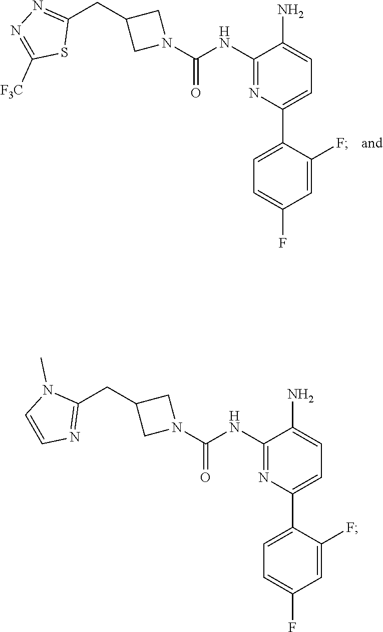 [SMILES: Nc1ccc(-c2ccc(F)cc2F)nc1NC(=O)N1CC(Cc2nnc(C(F)(F)F)s2)C1 ; Cn1ccnc1CC1CN(C(=O)Nc2nc(-c3ccc(F)cc3F)ccc2N)C1]
or a pharmaceutically acceptable salt thereof.

24. The compound of claim 1, wherein the compound is selected from: 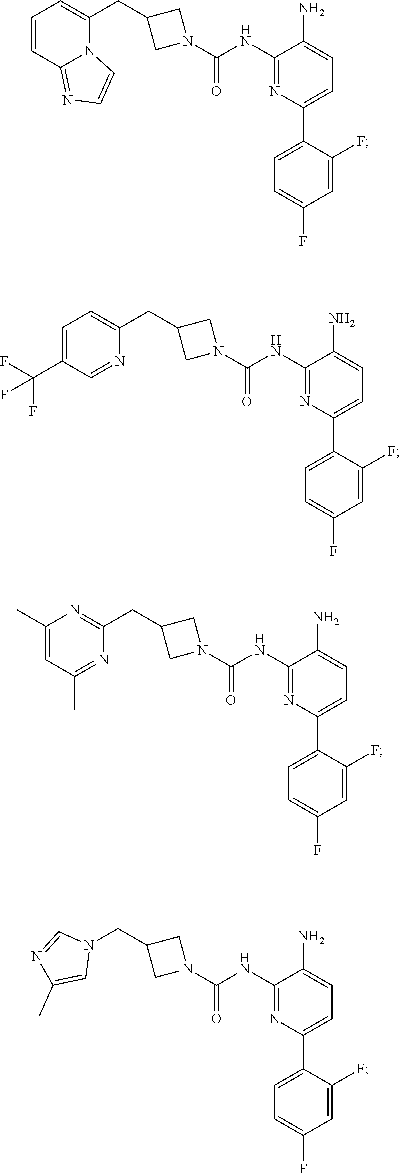 [SMILES: Nc1ccc(-c2ccc(F)cc2F)nc1NC(=O)N1CC(Cc2cccc3nccn23)C1 ; Nc1ccc(-c2ccc(F)cc2F)nc1NC(=O)N1CC(Cc2ccc(C(F)(F)F)cn2)C1 ; Cc1cc(C)nc(CC2CN(C(=O)Nc3nc(-c4ccc(F)cc4F)ccc3N)C2)n1 ; Cc1cn(CC2CN(C(=O)Nc3nc(-c4ccc(F)cc4F)ccc3N)C2)cn1] 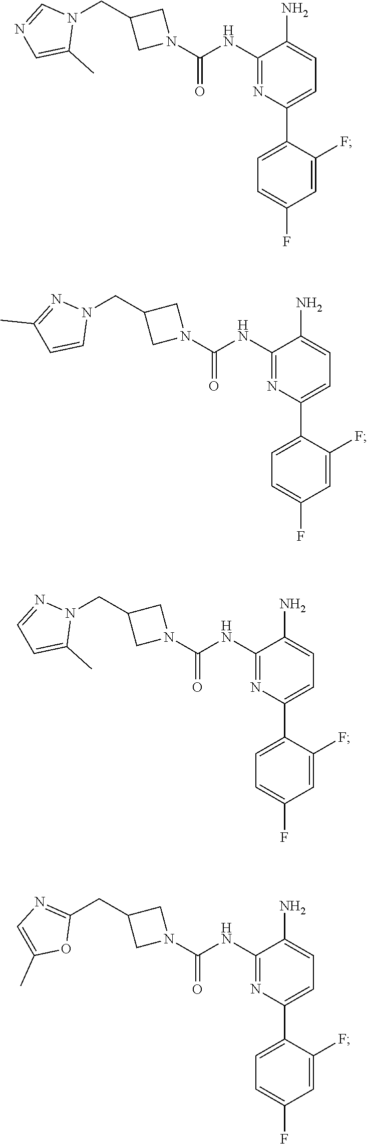 [SMILES: Cc1cncn1CC1CN(C(=O)Nc2nc(-c3ccc(F)cc3F)ccc2N)C1 ; Cc1ccn(CC2CN(C(=O)Nc3nc(-c4ccc(F)cc4F)ccc3N)C2)n1 ; Cc1ccnn1CC1CN(C(=O)Nc2nc(-c3ccc(F)cc3F)ccc2N)C1 ; Cc1cnc(CC2CN(C(=O)Nc3nc(-c4ccc(F)cc4F)ccc3N)C2)o1] 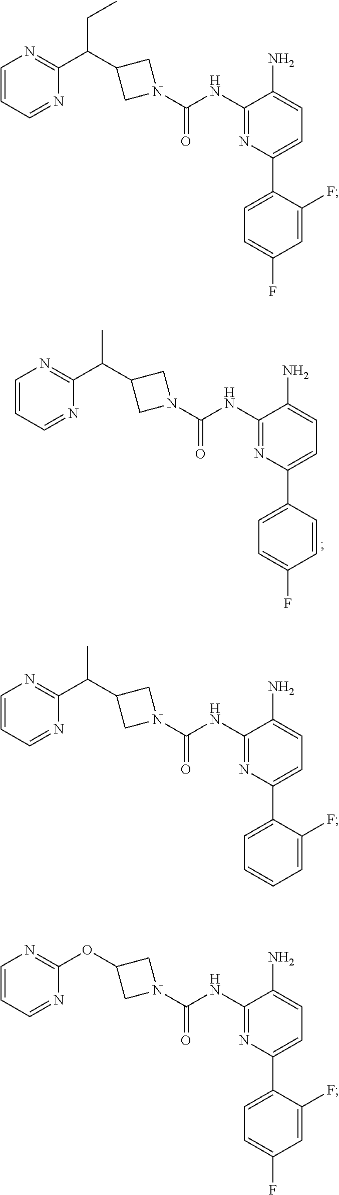 [SMILES: CCC(c1ncccn1)C1CN(C(=O)Nc2nc(-c3ccc(F)cc3F)ccc2N)C1 ; CC(c1ncccn1)C1CN(C(=O)Nc2nc(-c3ccc(F)cc3)ccc2N)C1 ; CC(c1ncccn1)C1CN(C(=O)Nc2nc(-c3ccccc3F)ccc2N)C1 ; Nc1ccc(-c2ccc(F)cc2F)nc1NC(=O)N1CC(Oc2ncccn2)C1] 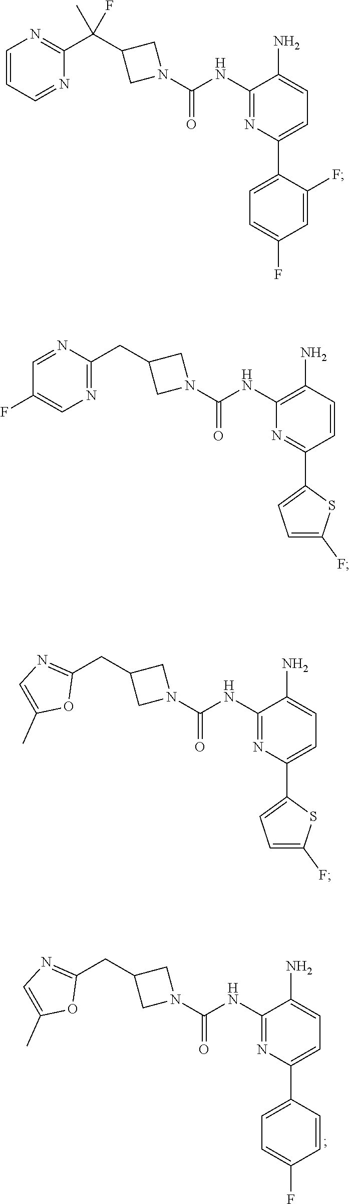 [SMILES: CC(F)(c1ncccn1)C1CN(C(=O)Nc2nc(-c3ccc(F)cc3F)ccc2N)C1 ; Nc1ccc(-c2ccc(F)s2)nc1NC(=O)N1CC(Cc2ncc(F)cn2)C1 ; Cc1cnc(CC2CN(C(=O)Nc3nc(-c4ccc(F)s4)ccc3N)C2)o1 ; Cc1cnc(CC2CN(C(=O)Nc3nc(-c4ccc(F)cc4)ccc3N)C2)o1] 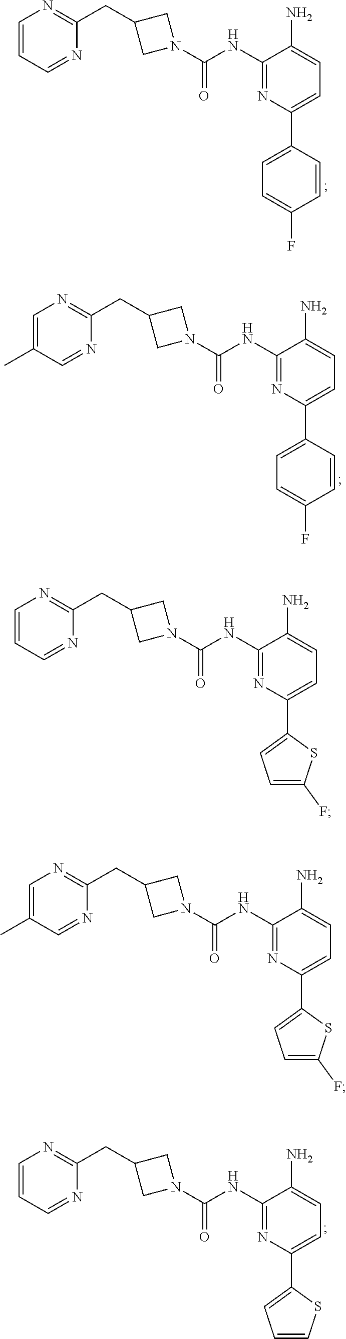 [SMILES: Nc1ccc(-c2ccc(F)cc2)nc1NC(=O)N1CC(Cc2ncccn2)C1 ; Cc1cnc(CC2CN(C(=O)Nc3nc(-c4ccc(F)cc4)ccc3N)C2)nc1 ; Nc1ccc(-c2ccc(F)s2)nc1NC(=O)N1CC(Cc2ncccn2)C1 ; Cc1cnc(CC2CN(C(=O)Nc3nc(-c4ccc(F)s4)ccc3N)C2)nc1 ; Nc1ccc(-c2cccs2)nc1NC(=O)N1CC(Cc2ncccn2)C1] 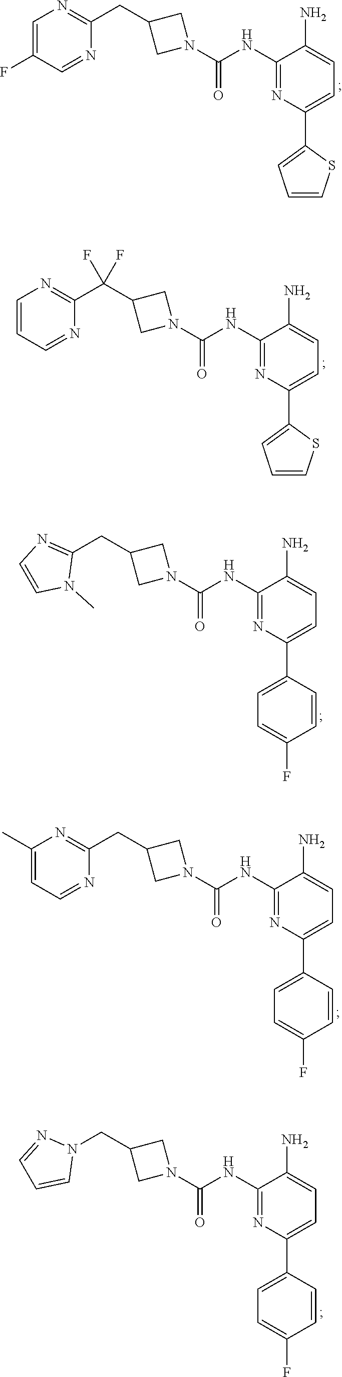 [SMILES: Nc1ccc(-c2cccs2)nc1NC(=O)N1CC(Cc2ncc(F)cn2)C1 ; Nc1ccc(-c2cccs2)nc1NC(=O)N1CC(C(F)(F)c2ncccn2)C1 ; Cn1ccnc1CC1CN(C(=O)Nc2nc(-c3ccc(F)cc3)ccc2N)C1 ; Cc1ccnc(CC2CN(C(=O)Nc3nc(-c4ccc(F)cc4)ccc3N)C2)n1 ; Nc1ccc(-c2ccc(F)cc2)nc1NC(=O)N1CC(Cn2cccn2)C1] 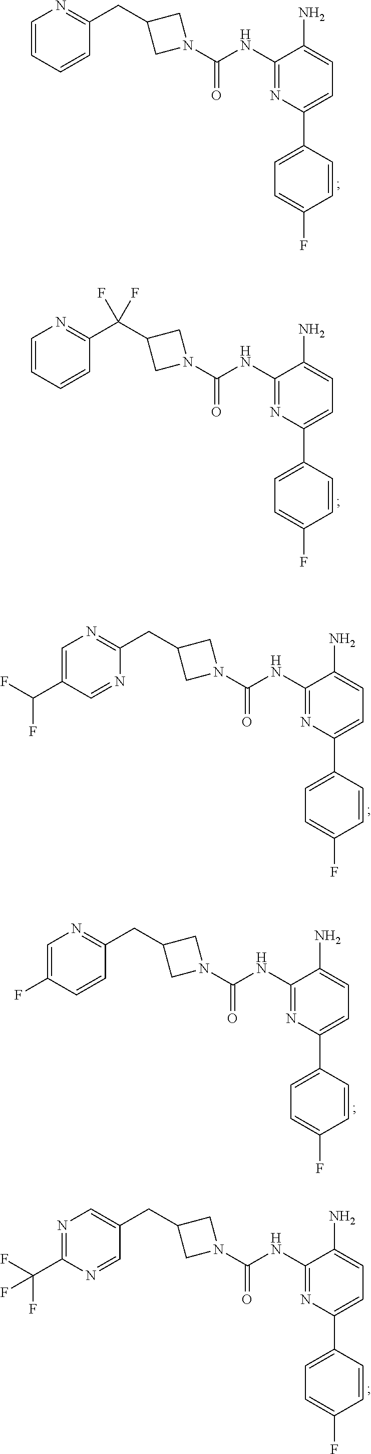 [SMILES: Nc1ccc(-c2ccc(F)cc2)nc1NC(=O)N1CC(Cc2ccccn2)C1 ; Nc1ccc(-c2ccc(F)cc2)nc1NC(=O)N1CC(C(F)(F)c2ccccn2)C1 ; Nc1ccc(-c2ccc(F)cc2)nc1NC(=O)N1CC(Cc2ncc(C(F)F)cn2)C1 ; Nc1ccc(-c2ccc(F)cc2)nc1NC(=O)N1CC(Cc2ccc(F)cn2)C1 ; Nc1ccc(-c2ccc(F)cc2)nc1NC(=O)N1CC(Cc2cnc(C(F)(F)F)nc2)C1] 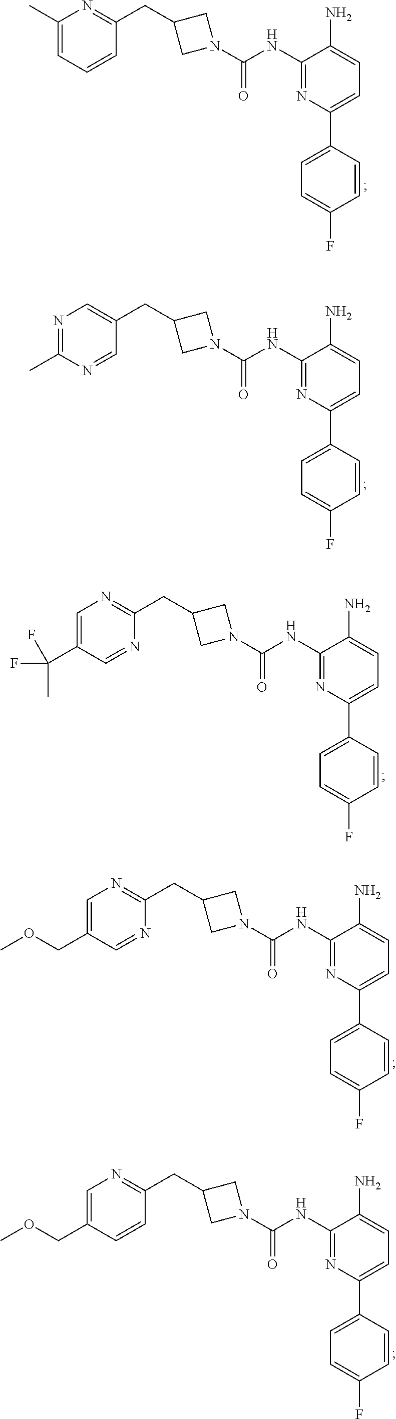 [SMILES: Cc1cccc(CC2CN(C(=O)Nc3nc(-c4ccc(F)cc4)ccc3N)C2)n1 ; Cc1ncc(CC2CN(C(=O)Nc3nc(-c4ccc(F)cc4)ccc3N)C2)cn1 ; CC(F)(F)c1cnc(CC2CN(C(=O)Nc3nc(-c4ccc(F)cc4)ccc3N)C2)nc1 ; COCc1cnc(CC2CN(C(=O)Nc3nc(-c4ccc(F)cc4)ccc3N)C2)nc1 ; COCc1ccc(CC2CN(C(=O)Nc3nc(-c4ccc(F)cc4)ccc3N)C2)nc1] 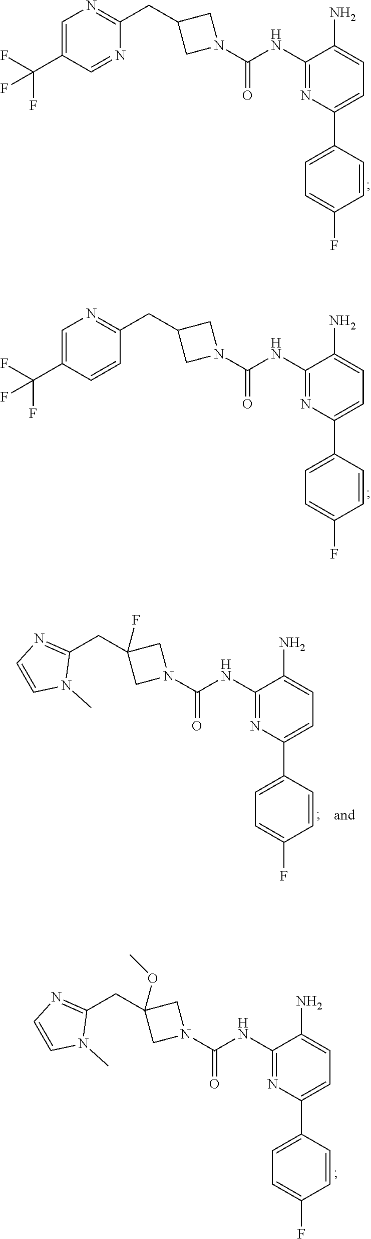 [SMILES: Nc1ccc(-c2ccc(F)cc2)nc1NC(=O)N1CC(Cc2ncc(C(F)(F)F)cn2)C1 ; Nc1ccc(-c2ccc(F)cc2)nc1NC(=O)N1CC(Cc2ccc(C(F)(F)F)cn2)C1 ; Cn1ccnc1CC1(F)CN(C(=O)Nc2nc(-c3ccc(F)cc3)ccc2N)C1 ; COC1(Cc2nccn2C)CN(C(=O)Nc2nc(-c3ccc(F)cc3)ccc2N)C1]
or a pharmaceutically acceptable salt thereof.

25. A composition comprising a compound of claim 1, or a pharmaceutically acceptable salt thereof; and a pharmaceutically acceptable carrier.

26. A method of inhibiting HD AC activity in a subject comprising the step of administering to the subject in need thereof an effective amount of a compound of claim 1, or a pharmaceutically acceptable salt thereof.

27. A method of treating a condition in a subject selected from a neurological disorder, memory or cognitive function disorder or impairment, extinction learning disorder, fungal disease or infection, inflammatory disease, hematological disease, psychiatric disorders, and neoplastic disease, comprising administering to the subject in need thereof an effective amount the compound of claim 1, or a pharmaceutically acceptable salt thereof.

28. The method of claim 27, wherein the condition is:
a. a cognitive function disorder or impairment associated with Alzheimer's disease, posterior cortical atrophy, normal-pressure hydrocephalus, Huntington's disease, seizure induced memory loss, schizophrenia, Rubinstein Taybi syndrome, Rett Syndrome, depression, Fragile X, Lewy body dementia, stroke, vascular dementia, vascular cognitive impairment (VCI), Binswanger's Disease, fronto-temporal lobar degeneration (FTLD), ADHD, dyslexia, major depressive disorder, bipolar disorder and social, cognitive and learning disorders associated with autism, traumatic brain injury (TBI), chronic traumatic encephalopathy (CTE), multiple sclerosis (MS), attention deficit disorder, anxiety disorder, conditioned fear response, panic disorder, obsessive compulsive disorder, posttraumatic stress disorder (PTSD), phobia, social anxiety disorder, substance dependence recovery, Age Associated Memory Impairment (AAMI), Age Related Cognitive Decline (ARCD), ataxia, Parkinson's disease, or Parkinson's disease dementia; or
b. a hematological disease selected from acute myeloid leukemia, acute promyelocytic leukemia, acute lymphoblastic leukemia, chronic myelogenous leukemia, myelodysplastic syndromes, and sickle cell anemia; or
c. a neoplastic disease; or
d. a disorder of learning extinction selected from fear extinction and post-traumatic stress disorder; or
e. hearing loss or a hearing disorder; or
f. fibrotic diseases, such as pulmonary fibrosis, renal fibrosis, cardiac fibrosis, and scleroderma; or
g. bone pain in patients with cancer; or
h. neuropathic pain.

29. The method of claim 28, wherein the condition is Alzheimer's disease, Huntington's disease, frontotemporal dementia, Friedreich's ataxia, post-traumatic stress disorder (PTSD), Parkinson's disease, or substance dependence recovery.

30. The method of claim 27, wherein the condition is selected from Alzheimer's disease, Huntington's disease, fronto-temporal lobar degeneration, Friedreich's ataxia, post-traumatic stress disorder, Parkinson's disease, Parkinson's disease dementia, substance dependence recovery, memory or cognitive function disorder or impairment, neurological disorder with synaptic pathology, disorder of learning distinction, psychiatric disorders, cognitive function or impairment associated with Alzheimer's disease, Lewy body dementia, schizophrenia, Rubinstein Taybi syndrome, Rett Syndrome, Fragile X, multiple sclerosis, age associated memory impairment, age related cognitive decline, and social, cognitive and learning disorders associated with autism.
